SEQUENCE LISTING (6 sequences)

SEQ ID NO: 1 - DNA, 25 bp, Artificial Sequence
  Synthetic construct
caccgcggtgcttgtacttgcggcg

SEQ ID NO: 2 - DNA, 25 bp, Artificial Sequence
  Synthetic construct
aaaccgccgcaagtacaagcaccgc

SEQ ID NO: 3 - DNA, 90 bp, Artificial Sequence
  Synthetic construct
ggcctcgcggagatgcccagctgcggtgcttgtacttgcggcgcggcgtaggtccggctcatcacctcctcactcgcctccgcgcagaga

SEQ ID NO: 4 - DNA, 25 bp, Artificial Sequence
  Synthetic construct
caccggcgtgcggagggatgtggcc

SEQ ID NO: 5 - DNA, 25 bp, Artificial Sequence
  Synthetic construct
aaacggccacatccctccgcacgcc

SEQ ID NO: 6 - DNA, 89 bp, Artificial Sequence
  Synthetic construct
gtagttccgccatcggacggaagccgaccggggcgtgcggagggtgataataatgaatattggtagggggggcccgggtggcgtcatgc

(19) United States
(12) Patent Application Publication
(10) Pub. No.: US 2021/0171576 A1
(43) Pub. Date: Jun. 10, 2021

(54) METHODS FOR TREATING CANCER WITH ACYLDEPSIPEPTIDE ANALOGS

(71) Applicants: Walid A. Houry, Toronto (CA); Robert A. Batey, Toronto (CA); Mark F. Mabanglo, Toronto (CA); Jordan D. Goodreid, Toronto (CA); Keith S. Wong, Toronto (CA)

(72) Inventors: Walid A. Houry, Toronto (CA); Robert A. Batey, Toronto (CA); Mark F. Mabanglo, Toronto (CA); Jordan D. Goodreid, Toronto (CA); Keith S. Wong, Toronto (CA)

(21) Appl. No.: 15/734,185

(22) PCT Filed: Jun. 3, 2019
(86) PCT No.: PCT/CA2019/050771

§ 371 Date: Dec. 1, 2020

Related U.S. Application Data
(60) Provisional application No. 62/679,448, filed on Jun. 1, 2018.
(60) Provisional application No. 62/680,302, filed on Jun. 4, 2018.

(51) Int. Cl. C07K 7/54; A61P 35/00

(52) U.S. Cl. CPC C07K 7/54; A61K 38/00; A61P 35/00

(57) ABSTRACT

Methods and uses for treating cancer in a subject are provided. In particular, the present disclosure provides methods and uses relating to treating a subject with cancer by activating human mitochondrial CIpP (HsCIpP). The present disclosure further provides methods and uses of an acyldepsipeptide (ADEP) analog that activates HsCIpP in the subject in need thereof.

### RELATED APPLICATIONS

This application claims priority to U.S. Provisional Patent Applications Nos. 62/679,448 filed on Jun. 1, 2018, and 62/680,302 filed on Jun. 4, 2018, the contents of which are hereby incorporated by reference in their entirety.

### FIELD

The disclosure relates to methods for treating cancer in a subject.

The disclosure provides methods and uses relating to treating a subject with cancer with an acyldepsipeptide analog.

BACKGROUND

The HsCIpXP complex is an ATP-dependent protease complex found in the mitochondrial matrix that plays an important role in mitochondrial protein quality control. HsCIpXP is composed of the serine protease HsCIpP and the AAA+ ATPase HsCIpX. Assembly of the complex involves the capping of the barrel-shaped HsCIpP tetradecamer on one or both ends by the HsCIpX hexamer (Kang et al., 2002). Protein degradation by CIpXP typically involves the recognition, binding, and unfolding of the substrate by CIpX. The unfolded polypeptide is then threaded through CIpX's central pore into the lumen of CIpP. Once inside, the polypeptide is hydrolysed by the 14 Ser-His-Asp proteolytic sites of CIpP, and the resultant fragments are expelled from CIpP (Olivares et al., 2016).

The interaction between CIpX and CIpP is partly stabilized by the dynamic docking of IGF loops of CIpX (E436 to G450 in HsCIpX; the triplet motif being L439-G440-F441) at specific hydrophobic pockets formed between neighbouring CIpP subunits (Amor et al., 2016). Small molecules that abrogate the CIpX-CIpP interaction have been characterized extensively for bacterial CIpXP. Among them, the acyldepsipeptides (ADEPs) (Brotz-Oesterhelt et al., 2005) bind CIpP with high affinity at the same site that normally accommodates the IGF loops of CIpX (Alexopoulos et al., 2012). The high-affinity binding of ADEP also keeps CIpP in a poorly understood activated conformation (Lee et al., 2010; Li et al., 2010). Collectively, these molecular changes allow free access for small peptides, molten globules and even folded proteins into the lumen of CIpP, causing an increase in degradation activity that is dysregulated, leading to bacterial cell death. Hence, the ADEPs are considered potential antibiotics.

In humans, HsCIpP has been shown to physically interact with numerous mitochondrial proteins involved in vital cellular processes such as energy metabolism, mitochondrial translation, mitochondrial protein import, metabolism of amino acids and cofactors, and maintenance of the mitochondrial proteome (Cole et al., 2015; Szczepanowska et al., 2016).

### SUMMARY

The present disclosure shows that acyldepsipeptide (ADEP) analogs activate human mitochondrial CIpP (HsCIpP) in cancer cells, which triggers intrinsic apoptotic pathway, thereby killing the cancer cells.

Accordingly, the present disclosure provides a method for treating a subject having cancer, comprising using or administering a therapeutically effective amount of an ADEP analog to the subject in need thereof. Also provided herein is use of a therapeutically effective amount of ADEP analog for treating a subject with cancer. Further provided is use of a therapeutically effective amount of ADEP analog in the manufacture of a medicament for treating subject with cancer. Even further provided is a therapeutically effective amount of ADEP analog for use in treating a subject with cancer.

In an embodiment, the ADEP analog is ADEP-01, ADEP-02, ADEP-03, ADEP-04, ADEP-05, ADEP-06, ADEP-07, ADEP-08, ADEP-09, ADEP-10, ADEP-11, ADEP-12, ADEP-13, ADEP-14, ADEP-15, ADEP-16, ADEP-17, ADEP-18, ADEP-19, ADEP-20, ADEP-21, ADEP-22, ADEP-23, ADEP-24, ADEP-25, ADEP-26, ADEP-27, ADEP-28, ADEP-29, ADEP-30, ADEP-31, ADEP-32, ADEP-33, ADEP-34, ADEP-35, ADEP-36, ADEP-37, ADEP-38, ADEP-39, ADEP-40, ADEP-41, ADEP-42, ADEP-43, ADEP-44, ADEP-45, or ADEP-46.

In another embodiment, the ADEP analog is ADEP-01, ADEP-02, ADEP-04, ADEP-06, ADEP-10, ADEP-13, ADEP-14, ADEP-15, ADEP-17, ADEP-20, ADEP-25, ADEP-28, ADEP-29, ADEP-30, ADEP-32, ADEP-37, ADEP-38, or ADEP-41.

In another embodiment, the ADEP analog is ADEP-14, ADEP-17, ADEP-28, ADEP-29, ADEP-30, ADEP-32, ADEP-37, or ADEP-38.

In another embodiment, the ADEP analog activates protease activity HsCIpP, and wherein the protease activity is at least 0.87 as measured by the relative degradation (RD) index at 25 μM of the ADEP analog, optionally at least 0.9, optionally at least 0.95.

In another embodiment, the protease activity is at least 0.74 as measured by the RD index at 5 μM of the ADEP analog, optionally at least 0.8, optionally at least 0.85.

In another embodiment, the protease activity is at least 0.2 as measured by the RD index at 1 μM of the ADEP analog, optionally at 0.5, optionally at least 0.75, optionally at least 0.8.

In another embodiment, the cancer is breast cancer, prostate cancer, colon cancer, liver cancer, uterus cancer, thyroid cancer, lung cancer, lymph nodes cancer, bladder cancer, ovary cancer, stomach cancer, testis cancer, brain cancer, skin cancer, pancreatic cancer, acute myeloid leukemia, kidney cancer, cervical cancer, osteosarcoma, or neuroblastoma.

In another embodiment, the cancer is breast cancer, kidney cancer, cervical cancer, osteosarcoma, or neuroblastoma.

In an embodiment, the cancer is metastatic.

In an embodiment, the metastatic cancer is breast cancer.

In another embodiment, the subject is human.

In another embodiment, the ADEP analog is administered subcutaneously, intraperitoneally, intravenously, topically, or orally.

Also provided is a compound for use in treating a subject having a cancer, wherein the compound is a therapeutically effective amount of an ADEP analog.

Also provided is an acyldepsipeptide (ADEP) analog, wherein the ADEP analog is ADEP-01, ADEP-02, ADEP-03, ADEP-04, ADEP-05, ADEP-06, ADEP-07, ADEP-08, ADEP-09, ADEP-10, ADEP-11, ADEP-12, ADEP-13, ADEP-14, ADEP-15, ADEP-16, ADEP-17, ADEP-18, ADEP-19, ADEP-20, ADEP-21, ADEP-22, ADEP-23, ADEP-24, ADEP-25, ADEP-26, ADEP-27, ADEP-28, ADEP-29, ADEP-30, ADEP-31, ADEP-32, ADEP-33, ADEP-34, ADEP-35, ADEP-36, ADEP-37, ADEP-38, ADEP-39, ADEP-40, ADEP-41, ADEP-42, ADEP-43, ADEP-44, ADEP-45, or ADEP-46, or a variant or a derivative thereof.

Other features and advantages of the present disclosure will become apparent from the following detailed description. It should be understood, however, that the detailed description and the specific Examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments are described below in relation to the drawings in which:

FIG. 1A-O show graphical representation of activity of ADEP analogs against HsCIpP. FIG. 1A shows RD25 scores of the 46 ADEP analogs tested. Standard deviations (SDs) are shown as error bars. FIG. 1B shows RD1 scores of 18 ADEP analogs showing the strongest activation of HsCIpP. Names of the five ADEP analogs selected for further experiments are bolded. FIG. 1C shows chemical structures of the five ADEP analogs selected for further experiments. FIG. 1D shows chemical structures of ADEP-01, ADEP-02, ADEP-03, and ADEP-04. FIG. 1 E shows chemical structures of ADEP-05, ADEP-06, ADEP-07, and ADEP-08. FIG. 1F shows chemical structures of ADEP-09, ADEP-10, ADEP-11, and ADEP-12. FIG. 1G shows chemical structures of ADEP-13, ADEP-14, ADEP-15, and ADEP-16. FIG. 1H shows chemical structures of ADEP-17, ADEP-18, ADEP-19, and ADEP-20. FIG. 1I shows chemical structures of ADEP-21, ADEP-22, ADEP-23, and ADEP-24. FIG. 1J shows chemical structures of ADEP-25, ADEP-26, ADEP-27, and ADEP-28. FIG. 1K shows chemical structures of ADEP-29, ADEP-30, ADEP-31, and ADEP-32. FIG. 1L chemical structures of ADEP-33, ADEP-34, ADEP-35, and ADEP-36. FIG. 1M chemical structures of ADEP-37, ADEP-38, ADEP-39, and ADEP-40. FIG. 1N chemical structures of ADEP-41, ADEP-42, ADEP-43, and ADEP-44. FIG. 1O Chemical structures of ADEP-45 and ADEP-46.

FIG. 2A-G show graphical representation of ADEPs enhancing both the peptidase and protease activity of HsCIpP. FIG. 2A shows peptidase activity of 8 μM HsCIpP (monomeric concentration) against different fluorogenic peptides (at 500 μM final concentration). FIG. 2B shows Michaelis-Menten kinetic analysis of Ac-WLA-AMC cleavage by 6 μM of HsCIpP. The kinetic parameters shown were derived from non-linear regression analysis of the initial rates. SDs of the activity data are shown as error bars. FIG. 2C shows Enhancement of the peptidase activity of 6 μM HsCIpP by ADEP-28 (left panel) and ADEP-41 (right panel). Ac-WLA-AMC was used at a final concentration of 100 μM. FIG. 2D shows enhancement of the protease activity of 3 μM HsCIpP by ADEP-28 (left panel) and ADEP-41 (right panel). FIG. 2E shows Vₘₐₓ, Hill coefficient (h), and the microscopic apparent dissociation constant (K_{0.5}) of all five ADEP analogs tested, derived from the non-linear regression analyses of data collected for the peptidase (left side of table) and protease (right side of table) activity of HsCIpP. Errors shown are from curve-fitting. FIG. 2F shows HsCIpP protease activity profiles while interacting with HsCIpX in the presence of ADEP-28, ADEP-06 and ADEP-02. Solid lines represent simple traces of the kinetic data. FIG. 2G shows observed minimal rates (mean±SD) (Vₘᵢₙ Observed) of casein-FITC degradation and the corresponding ADEP concentration at Vₘᵢₙ Observed. The kinetic data shown in each panel in FIG. 2C and FIG. 2D were derived from three independent replicates. SDs are shown as error bars.

FIG. 3A-G show graphical representation of enhancement of peptidase and protease activity of HsCIpP by ADEP-06, ADEP-02 and ADEP-04. FIG. 3A shows Michaelis-Menten kinetic analysis of Ac-WLA-AMC cleavage by 1.5 μM HsCIpP in the presence of 4.5 μM HsCIpX (upper panel) and by 2 μM EcCIpP (lower panel). In both experiments, the kinetic parameters shown were derived from non-linear regression analysis of the initial rates. Error bars shown correspond to standard deviations (SDs) derived from at least three independent replicates. Statistical errors shown for the calculated kinetic parameters correspond to curve-fitting errors. FIG. 3B shows enhancement of the peptidase activity of HsCIpP by ADEP-06, ADEP-02 and ADEP-04. FIG. 3C shows enhancement of the peptidase activity of HsCIpP by ADEP-06, ADEP-02 and ADEP-04. FIG. 3D shows enhancement of the protease activity of HsCIpP by ADEP-06, ADEP-02 or ADEP-04. FIG. 3E shows kinetic analysis of the degradation of casein-FITC by HsCIpP in the presence of HsCIpX at different molar ratios. Data points were analyzed by non-linear regression using the Hill equation. Relevant kinetic parameters derived from the analysis are as shown. FIG. 3F shows degradation of unlabelled casein by HsCIpX and HsCIpP in the absence (DMSO panels) and presence of ADEP-28 at 0.1 μM (middle panels) and 2 μM (right panels), observed on SDS-PAGE gels. The arrow to the right of creatine kinase panels indicates the correct band for creatine kinase. FIG. 3G shows densitometry analysis of casein illustrated in (F). Data points were derived from 3 independent replicates. The kinetic data shown in each panel in FIG. 3B to FIG. 3D were derived from three independent replicates.

FIG. 4A-F show graphical representation of ADEP inducing HsCIpP-dependent cytotoxicity in HEK293 T-REx cells. FIG. 4A shows cytotoxicity profiles of HEK293 T-REx WT (CLPP^{+/+}) and HEK293 T-REx CLPP cells treated with ADEP-28 (left panel) and ADEP-41 (right panel). Relative cell viability±SDs (shown as error bars) was calculated by normalizing data collected from 4 independent replicates to the DMSO-only control. FIG. 4B shows quantification of the cytotoxicity data collected for the five ADEP analogs tested. For WT, IC₅₀ values±curve-fitting errors were derived from non-linear regression analysis using a standard dose response equation. For CLPP^{-/-}, the lack of a minimum plateau in the cytotoxicity profiles prohibited non-linear regression analysis. FIG. 4C shows IC₅₀ values of the five ADEP analogs tested strongly correlate with their respective apparent binding affinity (K_{0.5}) to HsCIpP in vitro (shown in FIG. 2E) that were derived from the protease activity data. FIG. 4D shows Western blots on whole cell lysates of HEK293 T-REx WT, CLPP^{-/-}, HEK293 T-REx+CLPP-FLAG, and HEK293 T-REx+CLPP_{S153A}-FLAG. The absence or presence of Dox in the tissue culture is indicated as “−Dox” or “+Dox”, respectively. The primary antibody used in each blot is indicated to the right of each panel. Protein molecular weight markers are shown on the left. For the α-HsCIpP blot, band I corresponds to HsCIpP (WT or S153A mutant)-FLAG, band II corresponds to HsCIpP (WT or S153A mutant) that is missing the C-terminal FLAG-tag, and band Ill corresponds to endogenous HsCIpP. FIG. 4E shows ADEP-41 cytotoxicity profiles of HEK293 T-REx WT, CLPP^{-/-}, and WT cells that overexpress the C-terminal FLAG-tagged HsCIpP (+CLPP-FLAG) or the proteolytically inactive mutant (+CLPP_{S153A}-FLAG). Data collected on cells grown without Dox are shown on the left panel; data collected in the presence of 0.4 μg/mL Dox are shown on the right panel. FIG. 4F shows IC₅₀ values derived from the cytotoxicity profiles of (E). For HEK293 T-REx CLPP^{-/-}. IC₅₀ in FIG. 2C and FIG. 2F were estimated and are expressed in numerical ranges (denoted with *).

FIGS. 5A-D show graphical representation of ADEP-induced cytotoxicity partially dependent on cell type. FIG. 5A shows cytotoxicity profiles of HEK293 T-REx WT (CLPP^{+/+}) and HEK293 T-REx CLPP^{-/-} cells treated with

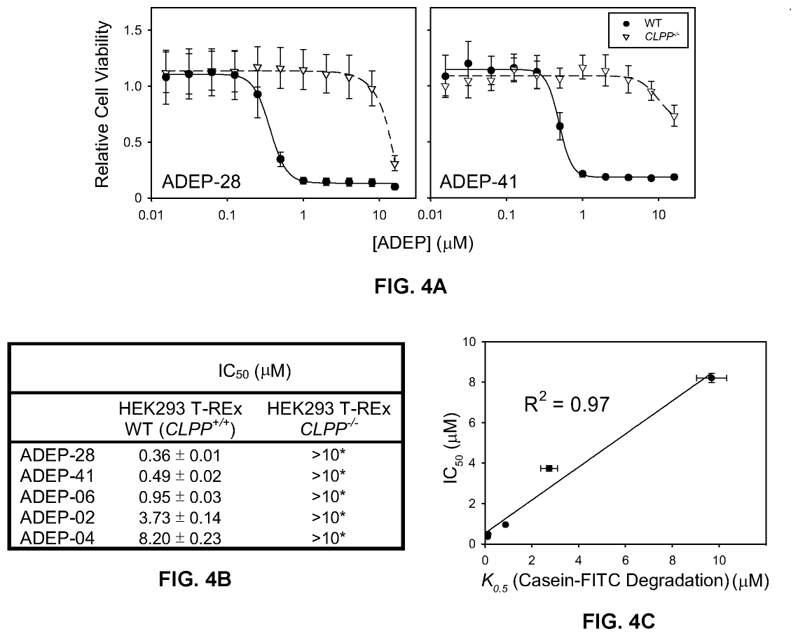

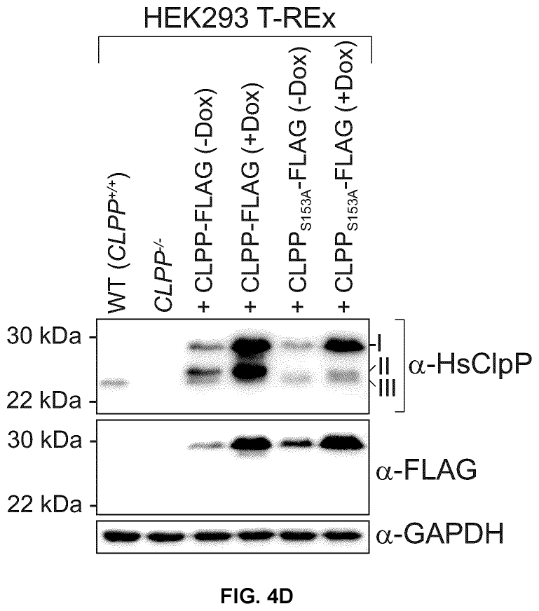

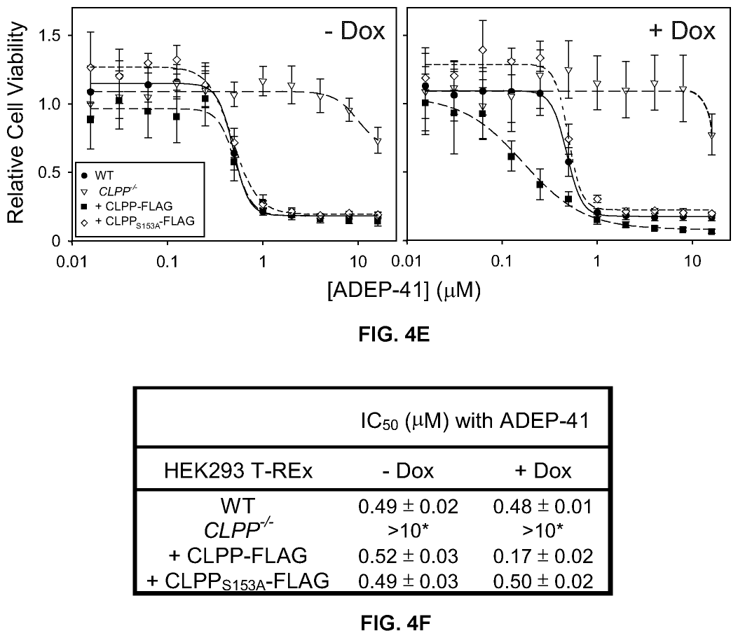

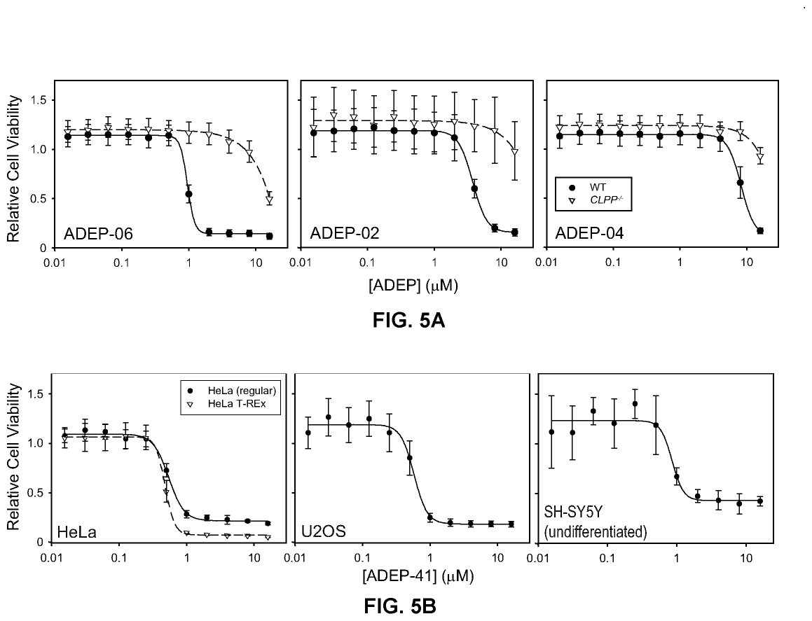

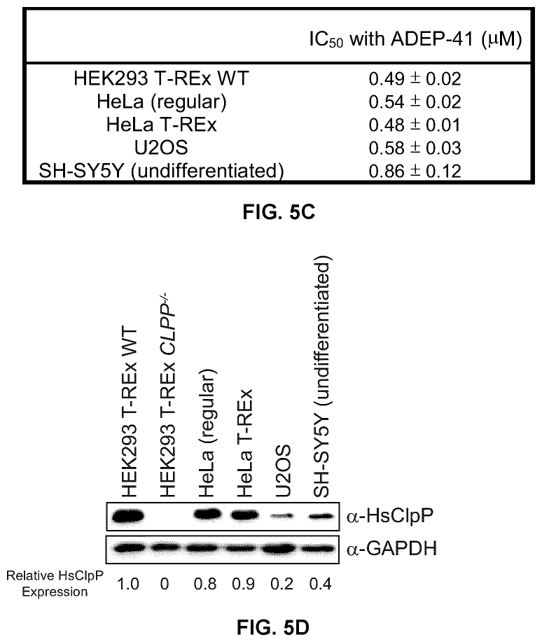

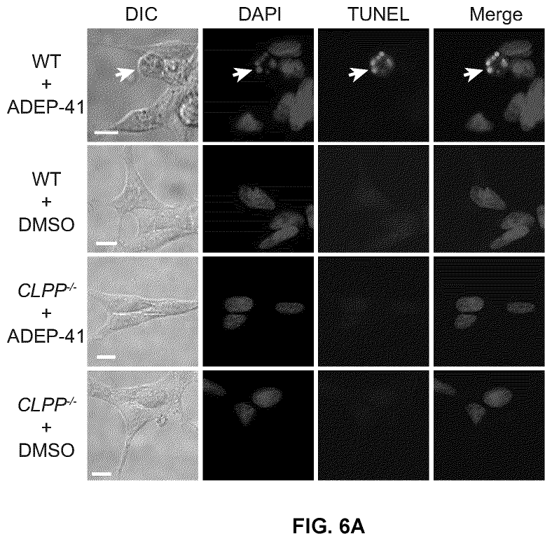

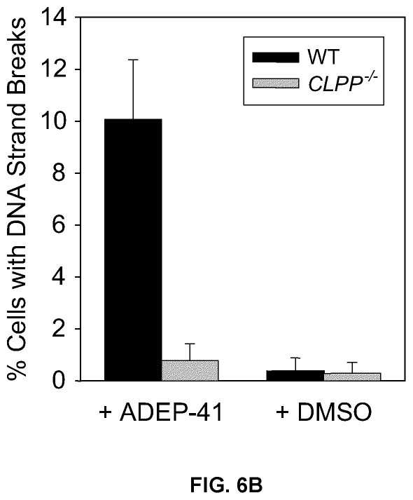

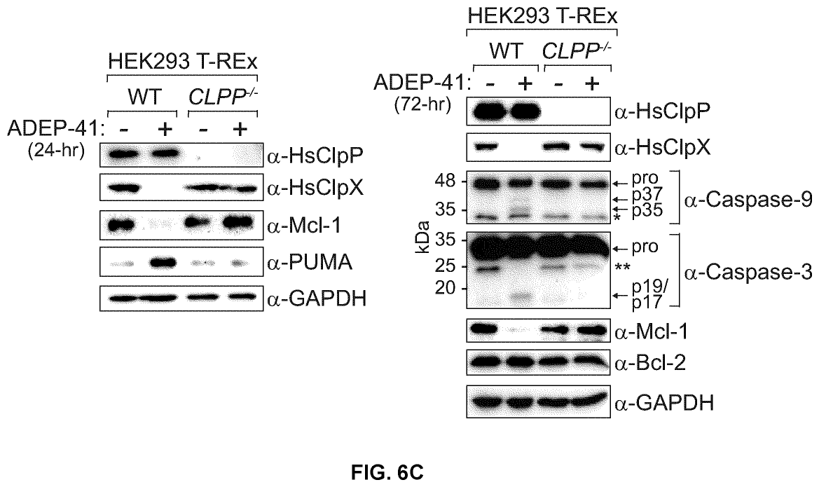

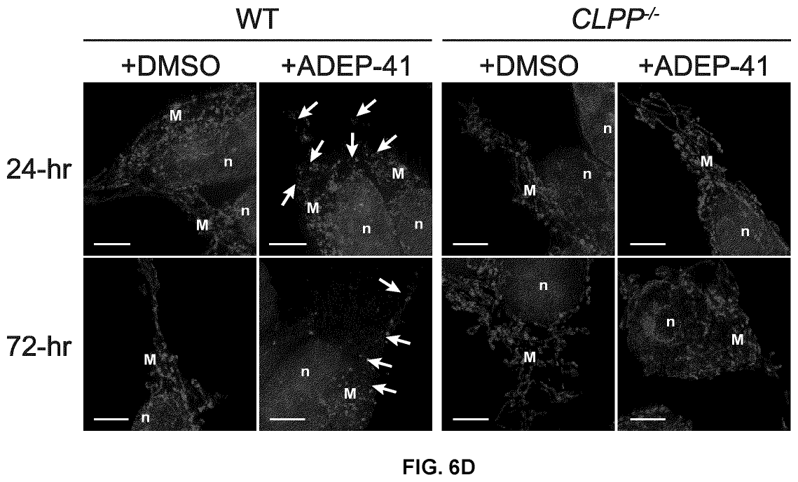

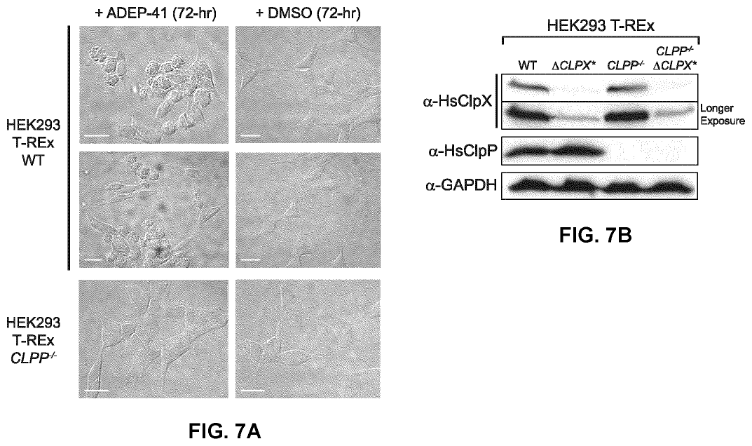

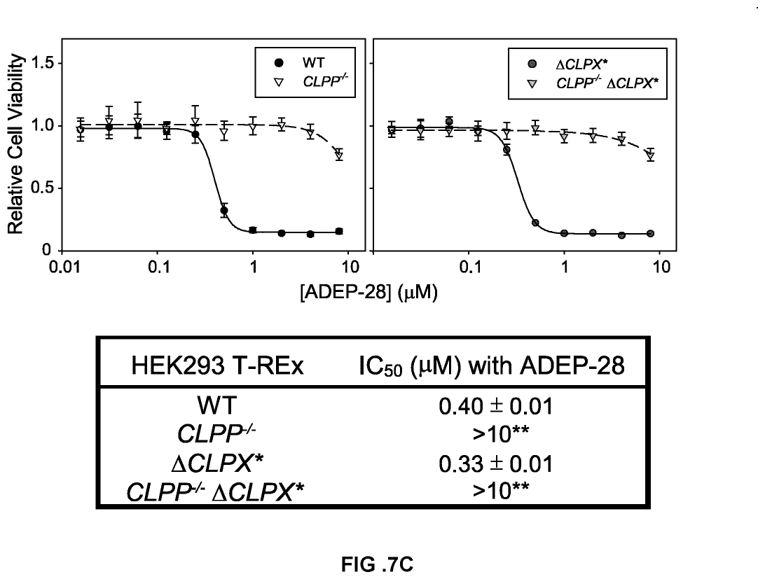

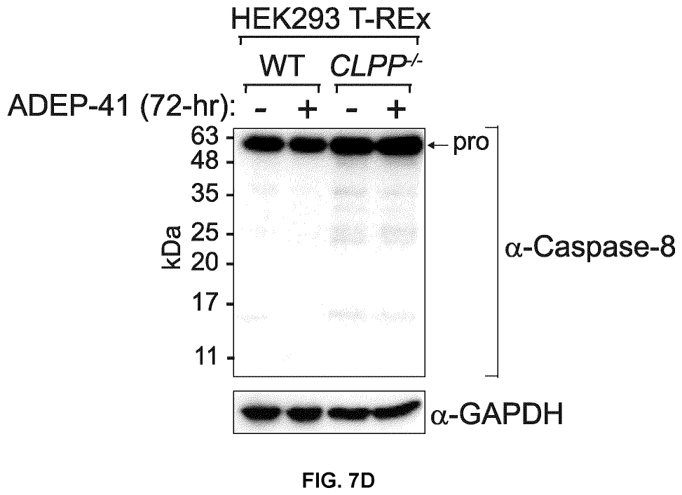

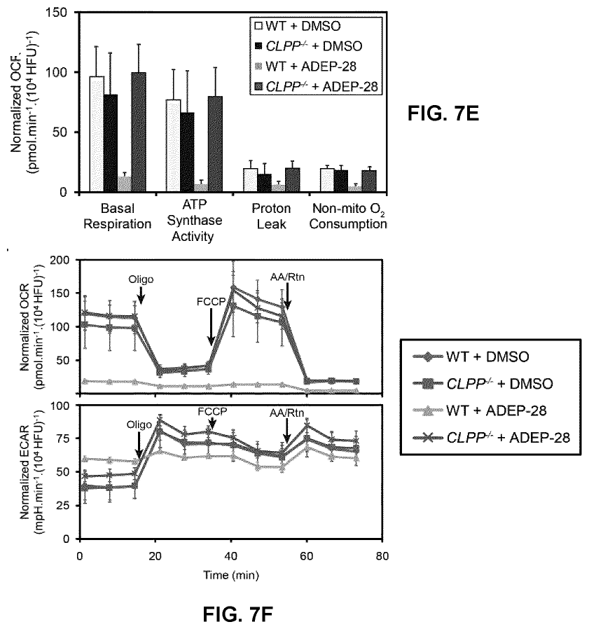

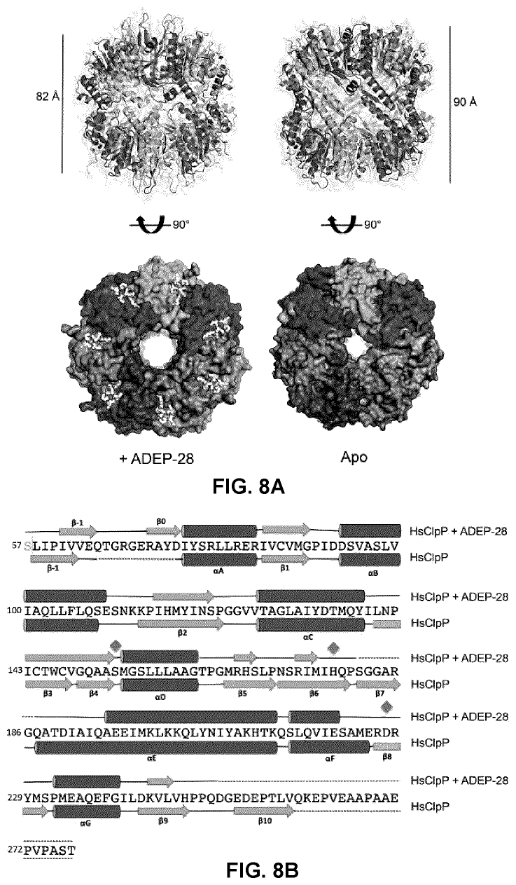

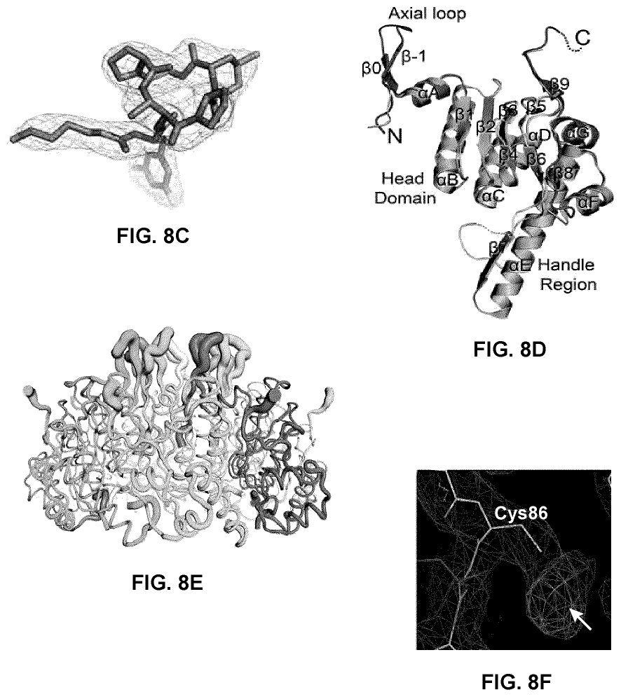

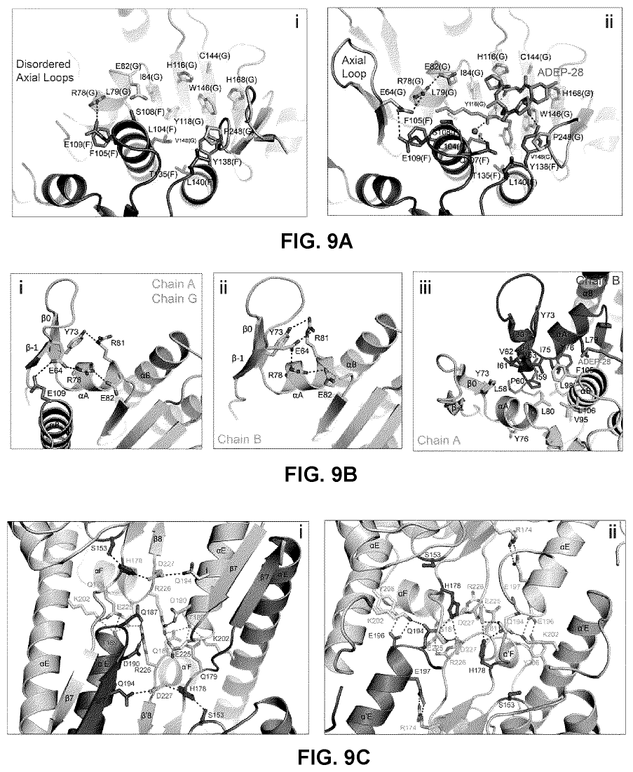

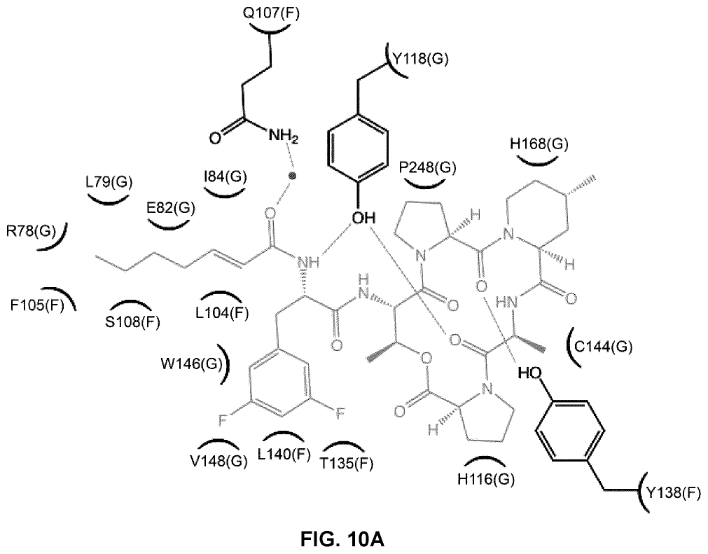

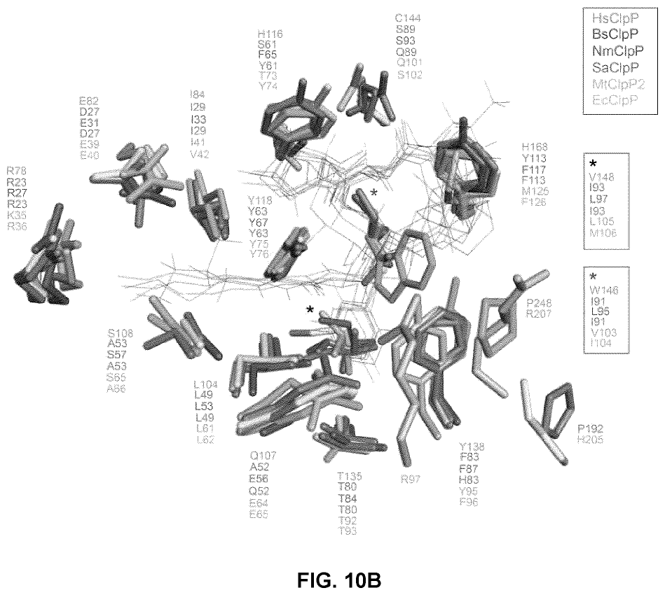

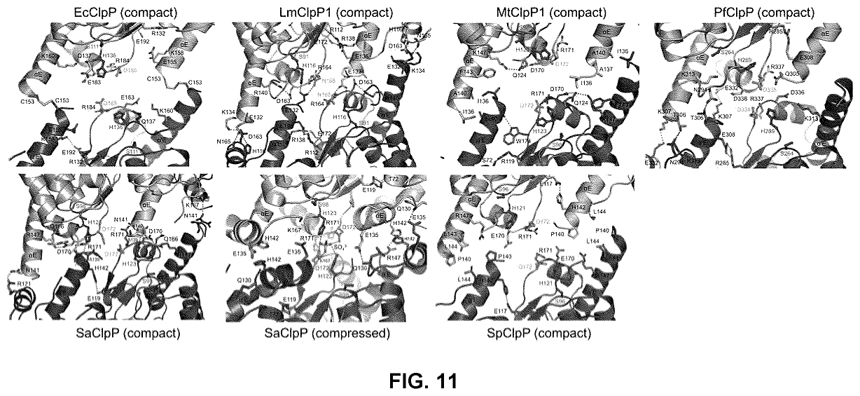

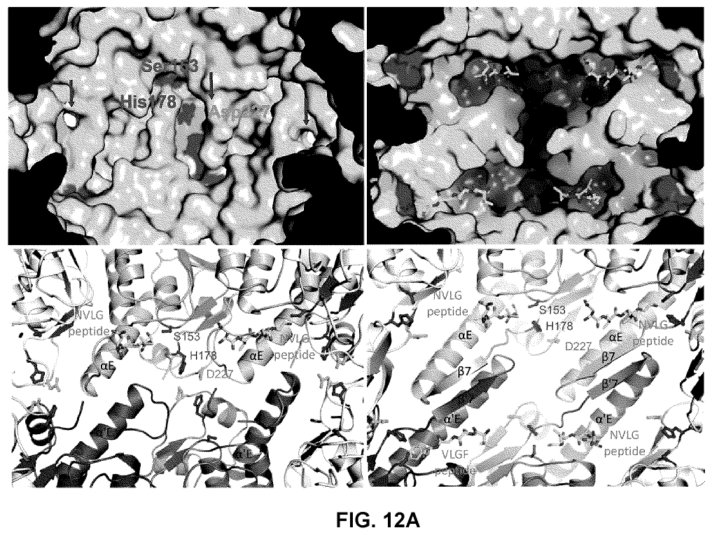

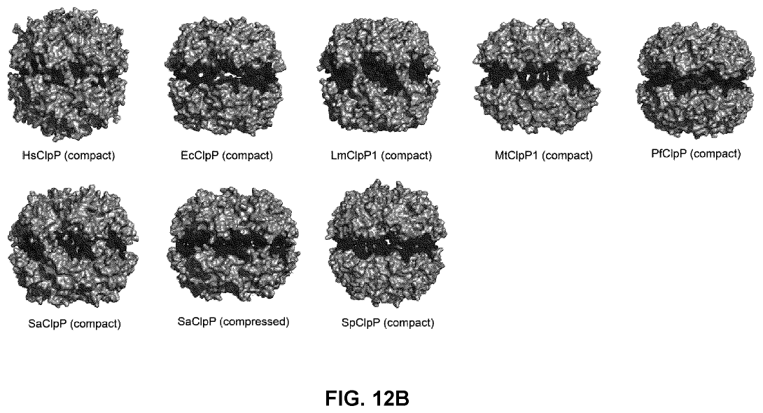

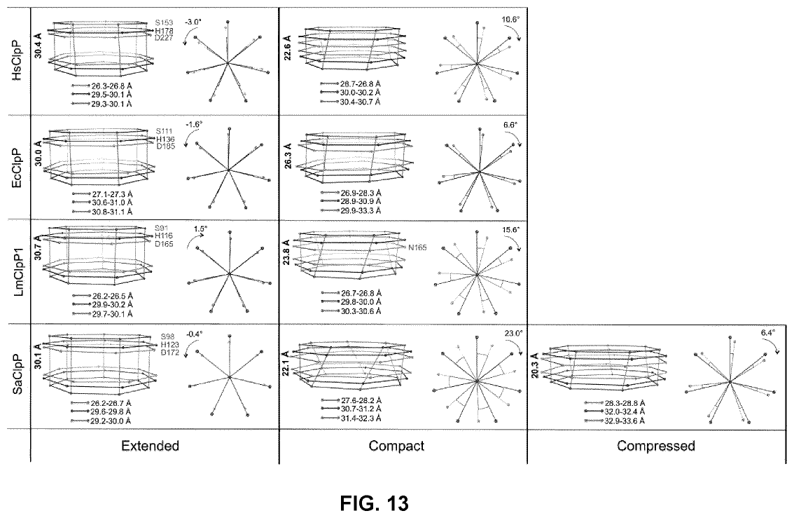

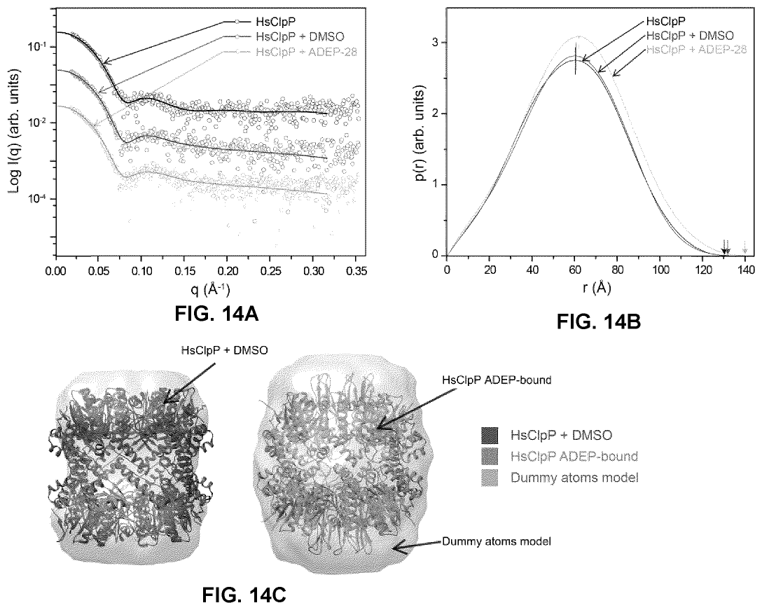

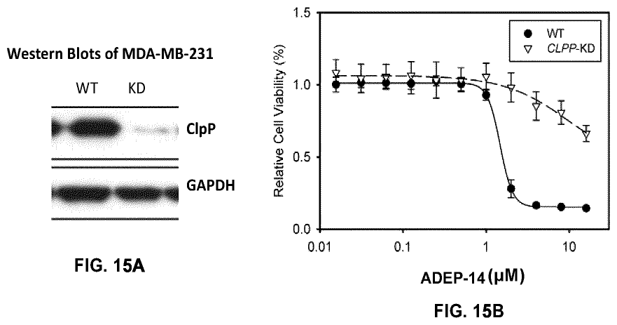

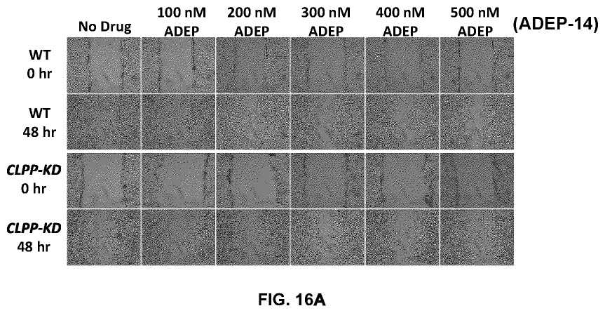

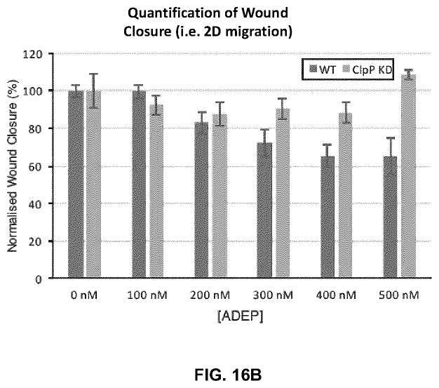

ADEP-06 (left panel), ADEP-02 (middle panel) and ADEP-04 (right panel). The relative cell viability was calculated by normalizing all data points to the DMSO-only control for each data set. FIG. 5B shows ADEP-41 cytotoxicity profiles of HeLa (regular) and HeLa T-REx (left panel), U2OS (middle panel) and undifferentiated SH-SYSY cells (right panel). Relative cell viability was calculated by normalizing all data points to the DMSO-only control for each data set. FIG. 5C shows IC₅₀ derived from the cytotoxicity profiles shown in FIG. 5B by non-linear regression analysis using the standard dose response equation. The IC₅₀ for HEK293 T-REx WT is identical to the one shown in FIG. 4B. FIG. 5D shows Western blot for endogenous HsCIpP expression in HEK 293 T-REx WT, HEK 293 T-REx CLPP^{-/-}, HeLa (regular), HeLa T-REx, U2OS, and undifferentiated SH-SY5Y. GAPDH is used as the loading control. The numbers at the bottom represent intracellular HsCIpP level in each cell line, relative to HEK293 T-REx WT. Data points in FIG. 5A and FIG. 5B were collected from 4 independent replicates.

FIGS. 6A-D show images and graphical representation of ADEP inducing intrinsic, caspase-dependent apoptosis. FIG. 6A shows light (DIC) and fluorescence (DAPI, TUNEL) microscopy images of HEK293 T-REx WT and HEK293 T-REx CLPP^{-/-} treated with 10 μM of ADEP-41 or DMSO for 72 hours. The DAPI + TUNEL (Merge) images are shown on the far-right panels. WT cells showing fragmentation of chromosomal DNA and presence of DNA strand breaks (i.e. TUNEL-positive) are marked with white arrows. The scale bars in the DIC panels represent a length of 50 μm. FIG. 6B shows quantification of WT and CLPP^{-/-} cells treated with ADEP-41 or DMSO that have acquired DNA strand breaks as observed in the TUNEL assay. SDs of three independent replicates are shown. FIG. 6C shows Western blots for HsCIpP, HsCIpX and major marker proteins for caspase-dependent apoptosis, on whole cell lysates of WT and CLPP^{-/-} cells treated with 20 μM of ADEP-41 for 24 hours (left panels) or 10 μM of the compound for 72 hours (right panels). “pro” denotes the proform of the caspases. “*” denotes a cross-reacting protein band with the α-Caspase-9 antibody; “**” denotes a possibly non-activated intermediate of Caspase-3 processing. GAPDH was used as a loading control. The primary antibodies used in each blot are indicated to the right of each panel. Protein molecular weight markers are shown on the left. FIG. 6D shows 3DSIM analysis of mitochondrial morphology in WT and CLPP cells during apoptosis. Cells were treated with 2 μM of ADEP-41 or DMSO for 24 or 72 hours prior to imaging. Mitochondria were stained with MitoTracker Red CMXRos and are observable as web-like structures that are indicated with the capital letter M. Fragmentation of mitochondria is indicated with arrows pointing at selected fragments. Nuclei were stained with Hoechst 33342 and are observable as large oval entities that are indicated with the lower-case letter n. Representative images of at least three independent replicates are shown. Scale bars shown correspond to a length of 5 μm.

FIGS. 7A-F show images and Western blots showing activation of the intrinsic, caspase-dependent apoptosis in ADEP-induced cytotoxicity. FIG. 7A shows light microscopy on HEK293 T-REx WT (top 2 rows) and HEK293 T-REx CLPP^{-/-} cells (bottom row) treated with 10 μM of ADEP-41 (panels on left column) or DMSO (panels on right column) over 72 hours. The white bars shown are 100 μm. FIG. 6B shows Western blot for HsCIpX, HsCIpP and GAPDH on whole cell lysates of WT, [symbol] CLPX*, CLPP^{-/-} and CLPP^{-/-} [symbol] CLPX* cells. The blot for HsCIpX is shown with normal exposure time (upper panel of α-HsCIpX) and long exposure time (lower panel of α-HsCIpX). FIG. 6C shows cytotoxicity profiles of HEK293 T-REx WT and CLPP^{-/-} cells (upper left panel) and of [symbol] CLPX* and CLPP^{-/-} [symbol] CLPX* cells (upper right panel) treated with ADEP-28. The IC₅₀ values are shown in the table below. Relative cell viability was calculated by normalizing all data points to the DMSO-only control for each data set. Data points were collected from 4 independent replicates. For WT and ΔCLPX*, IC₅₀ values were derived from non-linear regression analysis using a standard dose response equation (see Methods). For CLPP^{-/-} and CLPP^{-/-31} ΔCLPX*, the lack of a minimum plateau in the cytotoxicity profiles prohibited the derivation of IC₅₀ by non-linear regression analysis. Instead, IC₅₀ values were estimated and are expressed in numerical ranges (denoted by **). FIG. 6D shows Western blot for Caspase-8 on whole cell lysates of WT and CLPP^{-/-} cells treated with 10 μM ADEP-41 for 72 hours. “pro” denotes the pro form of Caspase-8. Protein molecular weight markers are shown on the left. FIG. 6E shows respiratory (OXPHOS) profiles of WT and CLPP^{-/-} cells during apoptosis. Cell were treated with 1 μM of ADEP-28 or DMSO prior to metabolic measurements. Cellular oxygen consumption rate (OCR) associated with basal respiration, ATP synthase activity, proton leak and non-mitochondrial oxygen consumption (Non-mito O₂ consumption) were calculated using normalized OCR data from at least three independent replications shown in FIG. 6F (upper panel) and are shown with ±SDs. FIG. 6F shows original OXPHOS data of WT and CLPP^{-/-}0 cells after a 24-hour treatment with DMSO or ADEP-28. Injection of oligomycin (Oligo), FCCP and the mixture of antimycin A and rotenone (AA/Rtn) to each sample during the experiment was done at the specific time points as shown. Both OCR (upper panel) and ECAR (lower panel) were normalized by Hoechst 33342 nuclear stain fluorescence that reflects cell count. Errors shown correspond to the SDs of individual data sets collected from at least three independently prepared replicates. GAPDH is used as the loading control in FIG. 6B and FIG. 6D.

FIGS. 8A-F show representation of ADEP-binding inducing conformational changes in the HsCIpP tetradecamer. FIG. 8A show the ADEP-28-bound (left) and apo form (right) of the HsCIpP tetradecamer, shown in side views (top) and top views (bottom). The bound ADEP-28 are shown as sticks. The distances shown were measured between the Cα atoms of E109 (helix αB) located on the surface of two apposing CIpP ring. FIG. 8B shows the sequence and the secondary structure of HsCIpP in the presence and absence of ADEP-28. The catalytic triad residues (S153, H178, and D227) are indicated with diamonds. Note that Ser57 was introduced due to cloning. FIG. 8C shows 2Fₒ-F_{c} electron density map of ADEP-28 contoured at 1.0 σ. FIG. 8D shows superposition of the monomeric structures of ADEP28-bound HsCIpP (PDB 6BBA, chain A) and apo HsCIpP (PDB 1TG6, chain A), viewed from within the HsCIpP lumen. FIG. 8E shows cartoon putty representation of a single ring of ADEP-28-HsCIpP complex with thicker tubes indicating higher B-factors. FIG. 8F shows 2Fo-Fc map (mesh) contoured at 1.0 σ, and Fo-Fc map (mesh indicted with white arrow) contoured at 3.5 σ showing covalent modification on the thiol group of Cys86 that is observed in each HsCIpP subunit.

FIGS. 9A-C show representation of ADEP-binding causing structural rearrangements within the HsCIpP cylinder. FIG. 9A shows local molecular interactions at the binding pocket between adjacent HsCIpP subunits F and G in the absence of ADEP are shown in (i). Local molecular interactions between ADEP-28 and residues in the binding pocket between two adjacent HsCIpP subunits F and G are shown in (ii). ADEP-28 and the amino acid residues of interest are shown as sticks. Non-covalent bonds are indicated with dotted lines. The axial loop is indicated. FIG. 9B shows ADEP-28 binding induces the ordering of N-terminal axial pore loops into β-hairpins due to interactions shown in the three panels. FIG. 9C shows molecular interactions at the equatorial plane of apo and ADEP-28-bound HsCIpP. The different HsCIpP subunits are distinguished by different shades of grey. All the relevant amino acid residues are shown as sticks. The catalytic triad residues are S153, H178 and D227. Secondary structures of each HsCIpP subunit are labelled as shown, with subunits from the opposite ring denoted with the prime mark (′). Non-covalent bonds between residues are indicated with dotted lines.

FIGS. 10A and B show representation of structural details of the ADEP-28 binding pocket in HsCIpP. FIG. 10A shows a cartoon representation based on LigPlot of the ADEP-28 interactions with amino acid residues from the hydrophobic binding pocket between the two adjacent F and G subunits in HsCIpP. FIG. 10B show superposition of the amino acid residues lining the ADEP-binding pockets of CIpP from: human mitochondria (HsCIpP; this disclosure), Bacillus subtilis (BsCIpP, PDB 3KTI (Lee et al., 2010)), Neisseria meningitidis (NmCIpP, PDB 5DKP (Goodreid et al., 2016)), Staphylococcus aureus (SaCIpP, PDB 5VZ2), Mycobacterium tuberculosis (MtClpP2; PDB 4U0G (Schmitz et al., 2014)) and Escherichia coli (EcCIpP; PDB 3MT6 (Li et al., 2010)). All amino acid residues are shown as sticks and are listed from top to bottom in the same order as their respective CIpP proteins. The ADEPs are shown as very thin sticks.

FIG. 11 shows representation of molecular interactions at the ring-ring interface of different compact and compressed CIpP structures. The structures shown are from E. coli (EcCIpP, 3HLN; contains an A153C mutation introducing a disulfide bridge between two apposing αE helices), L. monocytogenes (LmCIpP1, 4JCQ), M. tuberculosis (MtCIpP1, 2CE3), P. falciparum (PfCIpP, 2F6I), S. aureus (SaCIpP, 4EMM), S. aureus (SaCIpP, 3ST9), and S. pneumoniae (SpCIpP, 1Y7O, contains an A153P mutation). Four neighbouring subunits are represented as cartoons and different shades of grey in the same way as in FIGS. 9A-C, with the relevant residues shown as stick models.

FIGS. 12A and B show representation of the ADEP-28-HsCIpP complex showing the presence of equatorial side pores in different CIpP structures. FIG. 12A shows the structures are shown as viewed from the inside of the HsCIpP tetradecamer. Top left panel—In the compact ADEP-28-HsCIpP complex, the catalytic triad is distorted such that H178 and D227 form hydrogen bonding and/or electrostatic interactions with D227 and H178 residues of the apposing ring, respectively, securing the compact conformation. Distortion of the catalytic triad causes S153 to move away from H178 and D227. The side pore is located in close proximity to the catalytic site. Top right panel—In the extended apo-HsCIpP (PDB 1TG6), the active site surface is significantly different from that of the compact ADEP-28-HsCIpP structure. The catalytic triads are ordered and poised for catalysis of bound peptide substrates, here shown as sticks occupying the substrate-binding cavity. The peptide substrates are shown by superimposing the structure of H. pylori CIpP with bound NVLGFTQ peptide (PDB 2ZL2) (Kim and Kim, 2008) to HsCIpP structure. Bottom panels—Cartoon representation of the compact ADEP-28-HsCIpP complex and extended apo-HsCIpP, showing the relative positions of the catalytic triads and peptide substrates. Four HsCIpP subunits that form the interface are in shades of grey the same way as in FIGS. 9A-C and FIG. 11. In the compact ADEP-28-HsCIpP structure, the unraveling of two N-terminal turns of the αE helix forms a structured loop that impinges on the substrate binding site (bottom left panel). In contrast, in the apo-HsCIpP structure, the β7 strand and αE helix are fully formed and present a cavity for substrate peptide binding. FIG. 12B shows surface representation of compact and compressed CIpP structures from human (HsCIpP, this study), E. coli (EcCIpP, PDB 3HLN), L. monocytogenes (LmCIpP, PDB 4JCQ), M. tuberculosis (MtCIpP1, PDB 2CE3), P. falciparum (PfCIpP, PDB 2F6I), S. aureus (SaCIpP, PDB 4EMM), S. aureus (SaCIpP, PDB 3ST9), and S. pneumoniae (SpCIpP, PDB 1Y7O). Equatorial side pores of distinct sizes and shapes are indicated by black surfaces that are from residues forming the rims of these pores.

FIG. 13 shows representation of putative intermediates in the CIpP functional cycle represented by various CIpP structures from different species. Shown are the relative positions of the Ser-His-Asp catalytic triads in the extended and compact structures of CIpP from different species. The PDB IDs for the extended HsCIpP, EcCIpP, LmCIpP1, and SaCIpP are: 1TG6, 1YG6, 4JCR, and 3STA, respectively; while those for the compact are: this study, 3HLN, 4JCQ, and 4EMM, respectively. Also, there is a compressed structure of SaCIpP with PDB ID 3ST9. Note that the compact state of EcCIpP was the result of the introduction of disulfide bond between two αE helices of apposing rings due to the mutation of A153 to Cys. For LmCIpP1, the WT protein has Asn instead of Asp in the catalytic triad (residue 165) and is in the compact state. The N165D mutant adopts the extended state. The Cα atoms of the catalytic Ser, His, and Asp are shown as spheres in black or different shades of grey. The distances between the different Cα atoms are shown as lines in the. The numbers on the left refer to the distance between the plane formed by the Ser Cα atoms of one heptamer relative to the plane formed by the Ser Cα atoms in the apposing heptamer.

FIGS. 14A-C show graphical representation of small angle X-ray scattering data for HsCIpP. FIG. 14A shows scattering profiles (Log of scattering intensity as a function of the scattering vector q) of apo HsCIpP in buffer, in DMSO-containing buffer and in the presence of ADEP-28. Experimental curves (open circles) were fitted using the GNOM program (solid lines). HsCIpP+DMSO and HsCIpP+ADEP-28 curves were divided by 10 and 100, respectively, for visualization purposes. FIG. 14B shows particle distance distribution functions of HsCIpP in buffer, in DMSO-containing buffer, and in the presence of ADEP-28. The vertical lines indicate maximum p(r) and arrows indicate Dₘₐₓ. FIG. 14C shows the X-ray structures of apo HsCIpP (PDB: 1TG6) and ADEP-28-bound HsCIpP superimposed on the DAMs of HsCIpP+DMSO and HsCIpP+ADEP-28.

FIG. 15A and B show ADEP-14 are less toxic to cells with lower level of CIpP. FIG. 15A shows Western blots for HsCIpP. FIG. 15B shows cytotoxicity of ADEP-14 on MDA-MB-231 cells.

FIGS. 16A and B show ADEP impairing MDA-MB-231 cell migration in 2D culture. FIG. 16A shows scratch wound healing on MDA-MB-231 WT and CLPP-KD cells in the presence or absence of ADEP-14. FIG. 16B shows quantification of scratch wound closure data by measuring the area of scratch wounds.

### DETAILED DESCRIPTION

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, the singular forms “a”, “an” and “the” include plural references unless the content clearly dictates otherwise. The modifier “about” used in connection with a quantity is inclusive of the stated value and has the meaning dictated by the context (e.g., it includes the degree of error associated with measurement of the particular quantity). When referring to a period such as about a year or annually, it includes a range from 9 months to 15 months. All ranges disclosed herein are inclusive of the endpoints, and the endpoints are independently combinable with each other.

As used herein, the term “treating” and its derivatives, refer to improving the condition, such as reducing or alleviating symptoms associated with the condition or improving the prognosis or survival of the subject.

As used herein, the term “effective amount” or “therapeutically effective amount” means an amount effective, at dosages and for periods of time necessary to achieve the desired result. For example, in the context or treating cancer, an effective amount is an amount that, for example, induces remission, reduces tumor burden, and/or prevents tumor spread (e.g. spread by metastasis) or growth compared to the response obtained without administration of the compound. Effective amounts may vary according to factors such as the disease state, age, sex, weight of the subject. The amount of a given polypeptide that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The present inventors have identified acyldepsipeptide (ADEP) analogs as treatment for cancer. The ADEP analogs activate human mitochondrial CIpP (HsCIpP) in cancer cells, which triggers intrinsic apoptotic pathway, thereby killing the cancer cells.

Accordingly, the present disclosure provides a method for treating a subject having cancer, comprising using or administering a therapeutically effective amount of an ADEP analog to the subject in need thereof. Also provided herein is use of a therapeutically effective amount of ADEP analog for treating a subject with cancer. Further provided is use of a therapeutically effective amount of ADEP analog in the manufacture of a medicament for treating subject with cancer. Even further provided is a therapeutically effective amount of ADEP analog for use in treating a subject with cancer.

The disclosure describes a number of ADEP analogs that are useful for treating cancer. In an embodiment, the methods and uses described herein comprise administering or using an ADEP analog, wherein the ADEP analog is ADEP-01, ADEP-02, ADEP-03, ADEP-04, ADEP-05, ADEP-06, ADEP-07, ADEP-08, ADEP-09, ADEP-10, ADEP-11, ADEP-12, ADEP-13, ADEP-14, ADEP-15, ADEP-16, ADEP-17, ADEP-18, ADEP-19, ADEP-20, ADEP-21, ADEP-22, ADEP-23, ADEP-24, ADEP-25, ADEP-26, ADEP-27, ADEP-28, ADEP-29, ADEP-30, ADEP-31, ADEP-32, ADEP-33, ADEP-34, ADEP-35, ADEP-36, ADEP-37, ADEP-38, ADEP-39, ADEP-40, ADEP-41, ADEP-42, ADEP-43, ADEP-44, ADEP-45, or ADEP-46. In another embodiment, the ADEP analog is ADEP-01, ADEP-02, ADEP-04, ADEP-06, ADEP-10, ADEP-13, ADEP-14, ADEP-15, ADEP-17, ADEP-20, ADEP-25, ADEP-28, ADEP-29, ADEP-30, ADEP-32, ADEP-37, ADEP-38, or ADEP-41. In another embodiment, the ADEP analog is ADEP-14, ADEP-17, ADEP-28, ADEP-29, ADEP-30, ADEP-32, ADEP-37, or ADEP-38. In an aspect, the ADEP analogs described herein are useful for activating protease activity of HsCIpP

Usefulness of structural analogs of ADEP in cancer treatment was assessed by their ability to activate and dysregulate the protease activity of HsCIpP by measuring their relative degradation (RD) index. All ADEP analogs were first screened at 25 μM, and their RD25 scores were calculated as described in the Example. The analogs that achieved RD25≥0.87 were screened a second time at 5 μM, followed by calculation of their RD5 scores. The top analogs with RD5≥0.74 were screened a third time at 1 μM, followed by calculation of their RD1 scores.

In an embodiment, the methods and uses described herein comprise administering or using an ADEP analog, wherein the ADEP analog activates protease activity of HsCIpP, wherein the protease activity is at least 0.2, 0.3, 0.4, 0.5, 0.55, 0.6, 0.65, 0.7, 0.71, 0.72, 0.73, 0.74, 0.75, 0.76, 0.77, 0.78, 0.79, 0.8, 0.85, 0.86, 0.87, 0.88, 0.89, 0.90, 0.91, 0.92, 0.93, 0.94, 0.95, 0.96, 0.97, 0.98, or 0.99 as measured by the RD index at 25 μM, 5 μM or 1 μM of the ADEP analog, preferably at least 0.2, 0.74, or 0.87, optionally at least 0.5, 0.75, 0.8, 0.85, 0.9 or 0.95. In another embodiment, the protease activity is at least 0.87 as measured by the RD index at 25 μM of the ADEP analog, optionally at least 0.9, optionally at least 0.95. In another embodiment, the protease activity is at least 0.74 as measured by the RD index at 5 μM of the ADEP analog, optionally at least 0.8, optionally at least 0.85. In another embodiment, the protease activity is at least 0.2 as measured by the RD index at 1 μM of the ADEP analog, optionally at 0.5, optionally at least 0.75, optionally at least 0.8.

The analogs described herein are useful for treating cancer. For example, the analogs are useful for treating cancer expressing high level of HsCIpP, such as when a cancer cell expresses at least 1.5-fold level of HsCIpP mRNA or protein compared to normal cells. Breast cancer, prostate cancer, colon cancer, liver cancer, uterus cancer, thyroid cancer, lung cancer, lymph nodes cancer, bladder cancer, ovary cancer, stomach cancer, testis cancer, kidney cancer, cervical cancer, osteosarcoma, skin cancer, pancreatic cancer, brain cancer such as neuroblastoma, as well as non-solid cancer such as acute myeloid leukemia, all exhibit high HsCIpP expression. In an embodiment, the methods and uses described herein comprise administering or using an ADEP analog for treating cancer, wherein the cancer is breast cancer, prostate cancer, colon cancer, liver cancer, uterus cancer, thyroid cancer, lung cancer, lymph nodes cancer, bladder cancer, ovary cancer, stomach cancer, testis cancer, brain cancer, skin cancer, pancreatic cancer, acute myeloid leukemia, kidney cancer, cervical cancer, osteosarcoma, or neuroblastoma. In another embodiment, the cancer is non-solid cancer. In another embodiment, the non-solid cancer is acute myeloid leukemia. In another embodiment, the cancer is breast cancer, kidney cancer, cervical cancer, osteosarcoma, or neuroblastoma. In an embodiment, the cancer is breast cancer. In an embodiment, the breast cancer is Invasive ductal carcinoma. In another embodiment, the brain cancer is neuroblastoma. In an embodiment, the cancer is metastatic. In an embodiment, the metastatic cancer is breast cancer. In another embodiment, the cancer cell expresses at least 1.2, 1.3, 1.4, 1.5, 1.6, 1.7, 1.8, 1.9, 2, 2.5, 3, 3.5, 4, 4.5, 5, 6, 7, 8, 9 or 10-fold level of HsCIpP mRNA or protein compared to a normal cell, optionally at least 1.5-fold.

The term “breast cancer”, as used herein, refers to a cancer that develops from breast tissue. Breast cancers are classified by several grading systems. Breast cancer can be classified by its histological appearance. Breast cancers can be derived from the epithelium lining the ducts or lobules, and these cancers are classified as ductal or lobular carcinoma. Carcinoma in situ is growth of low-grade cancerous or precancerous cells within a particular tissue compartment such as the mammary duct without invasion of the surrounding tissue. In contrast, invasive carcinoma does not confine itself to the initial tissue compartment. Grading is also used for classification. Grading compares the appearance of the breast cancer cells to the appearance of normal breast tissue. Normal cells in an organ like the breast become differentiated, such that they take on specific shapes and forms that reflect their function as part of that organ. Cancerous cells lose that differentiation. In breast cancer, the cells that would normally line up in an orderly fashion make up the milk ducts become disorganized. Cell division becomes uncontrolled. Cell nuclei become less uniform. Breast cancer can also be classified by stages.

Breast cancer staging using the TNM system is based on the size of the tumor (T), whether the tumor has spread to the lymph nodes (N) in the armpits, and whether the tumor has metastasized (M) (i.e. spread to a more distant part of the body). Larger size, nodal spread, and metastasis have a larger stage number and a worse prognosis. The main stages are: Stage 0 is a pre-cancerous or marker condition, either ductal carcinoma in situ (DCIS) or lobular carcinoma in situ (LCIS); Stages 1-3 are within the breast or regional lymph nodes; and Stage 4 is “metastatic” cancer that has a less favorable prognosis since it has spread beyond the breast and regional lymph nodes. The ADEP analogs described herein are useful for treating breast cancer. The ADEP analogs described herein are also useful for treating or preventing metastatic cancer. Further, the ADEP analogs described herein are also useful for treating or preventing metastatic breast cancer.

The term “subject”, as used herein, refers to any individual who is the target of administration or treatment. The subject can be an animal, for example, a mammal, optionally a human. The term “patient” refers to a subject under the care or treatment of a health care professional. In an embodiment, the subject is human. In another embodiment, the subject is non-human animal. In another embodiment, the non-human animal is a pet or a farm-animal. In another embodiment, the non-human animal is an ovine, a bovine, an equine, a caprine, a porcine, a canine, a feline, a rabbit, a rodent or a non-human primate.

The ADEP analog of the present disclosure may be formulated into a pharmaceutical composition, such as by mixing with a suitable excipient, carrier, and/or diluent, by using techniques that are known in the art. For example, the ADEP analog can be administered or used in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the inhibitor may be combined with various sweetening or flavouring agents, colouring matter or dyes, with emulsifying and/or suspending agents and with diluents such as water, ethanol, propylene glycol and glycerin, and combinations thereof.

As used herein, the term “pharmaceutically acceptable carrier” is intended to include any and all solvents, dispersion media, coatings, isotonic and absorption delaying agents, and the like, compatible with pharmaceutical use or administration. Suitable carriers are described in the most recent edition of Remington's Pharmaceutical Sciences, a standard reference text in the field, which is incorporated herein by reference. Optional examples of such carriers or diluents include, but are not limited to, water, saline, ringer's solutions, dextrose solution, and 5% human serum albumin.

In one embodiment, the active ingredient is prepared with a carrier that will protect it against rapid elimination from the body, such as a sustained/controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

A pharmaceutical composition is formulated to be compatible with its intended route of use or administration. The use or administration of compound to a subject comprises ingestion, inhalation, injection, or topical application. The route of injection includes but not limited to intradermal, subcutaneous, intramuscular, intravenous, intraosseous, intraperitoneal, intrathecal, epidural, intracardiac, intraarticular, intracavernous, intravitreal, intracerebral, intracerebroventricular, or intraportal. Topical use or administration also includes transdermal application. In an embodiment, the methods and uses described herein comprises an ADEP analog, wherein the ADEP analog is injected, administered, or used via intradermal, subcutaneous, intramuscular, intravenous, intraosseous, intraperitoneal, intrathecal, epidural, intracardiac, intraarticular, intracavernous, intravitreal, intracerebral, intracerebroventricular, or intraportal route. In another embodiment, the ADEP analog is administered or used topically, optionally transdermally. In another embodiment, the ADEP analog is administered or used subcutaneously, intraperitoneally, intravenously, topically, or orally. In a specific embodiment, wherein the cancer is skin cancer, the ADEP analog is administered or used topically, optionally transdermally.

The methods of treating cancer described herein also include selecting a subject determined to have cancer cells comprising an increased level of expression of an HsCIpP gene product relative to a reference expression level. The reference expression level is an expression level of HsCIpP in a non-cancerous cell. The person skilled in art can readily recognize methods for measuring expression level of an HsCIpP gene product relative to the reference expression level, for example, by using Enzyme-linked immunosorbent assay (ELISA), protein microarray, immunoelectrophoresis, Western blotting, immunohistochemistry, High-performance liquid chromatography (HPLC), and/or mass spectrometry.

Also provided is a compound for use in treating a subject having a cancer, wherein the compound is a therapeutically effective amount of an acyldepsipeptide (ADEP) analog. In an embodiment, the ADEP analog activates protease activity of human mitochondrial CIpP (HsCIpP), and wherein the protease activity is at least 0.87 as measured by the relative degradation (RD) index at 25 μM of the ADEP analog, optionally at least 0.9, optionally at least 0.95. In another embodiment, the protease activity is at least 0.74 as measured by the RD index at 5 μM of the ADEP analog, optionally at least 0.8, optionally at least 0.85. In another embodiment, the protease activity is at least 0.2 as measured by the RD index at 5 μM of the ADEP analog, optionally at 0.5, optionally at least 0.75, optionally at least 0.8. In another embodiment, the ADEP analog is ADEP-01, ADEP-02, ADEP-04, ADEP-06, ADEP-10, ADEP-13, ADEP-14, ADEP-15, ADEP-17, ADEP-20, ADEP-25, ADEP-28, ADEP-29, ADEP-30, ADEP-32, ADEP-37, ADEP-38, or ADEP-41. In another embodiment, the ADEP analog is ADEP-14, ADEP-17, ADEP-28, ADEP-29, ADEP-30, ADEP-32, ADEP-37, or ADEP-38.

In an embodiment, the compound is for use in treating a cancer wherein the cancer is breast cancer, prostate cancer, colon cancer, liver cancer, uterus cancer, thyroid cancer, lung cancer, lymph nodes cancer, bladder cancer, ovary cancer, stomach cancer, testis cancer, brain cancer, skin cancer, pancreatic cancer, acute myeloid leukemia, kidney cancer, cervical cancer, osteosarcoma, or neuroblastoma. In another embodiment, the cancer is non-solid cancer. In another embodiment, the non-solid cancer is acute myeloid leukemia. In another embodiment, the cancer is breast cancer, kidney cancer, cervical cancer, osteosarcoma, or neuroblastoma. In an embodiment, the cancer is breast cancer. In an embodiment, the breast cancer is Invasive ductal carcinoma. In another embodiment, the brain cancer is neuroblastoma. In an embodiment, the cancer is metastatic. In an embodiment, the metastatic cancer is breast cancer. In another embodiment, the subject is human. In another embodiment, the subject is non-human animal. In another embodiment, the non-human animal is a pet or a farm-animal. In another embodiment, the non-human animal is an ovine, a bovine, an equine, a caprine, a porcine, a canine, a feline, a rabbit, a rodent or a non-human primate. In another embodiment, the ADEP analog is administered or used subcutaneously, intraperitoneally, intravenously, topically, or orally.

In another aspect, also provided is an acyldepsipeptide (ADEP) analog, wherein the ADEP analog is ADEP-01, ADEP-02, ADEP-03, ADEP-04, ADEP-05, ADEP-06, ADEP-07, ADEP-08, ADEP-09, ADEP-10, ADEP-11, ADEP-12, ADEP-13, ADEP-14, ADEP-15, ADEP-16, ADEP-17, ADEP-18, ADEP-19, ADEP-20, ADEP-21, ADEP-22, ADEP-23, ADEP-24, ADEP-25, ADEP-26, ADEP-27, ADEP-28, ADEP-29, ADEP-30, ADEP-31, ADEP-32, ADEP-33, ADEP-34, ADEP-35, ADEP-36, ADEP-37, ADEP-38, ADEP-39, ADEP-40, ADEP-41, ADEP-42, ADEP-43, ADEP-44, ADEP-45, or ADEP-46, or a variant or a derivative thereof.

The following non-limiting Examples are illustrative of the present disclosure:

### EXAMPLES

### Example 1: Identification of Acyldepsipeptide Analogs Dysregulate Human Mitochondrial CIpP (HsCIpP) Protease Activity and Cause Apoptotic Cell Death in Cancer Cells

### Introduction

The present Example describes the identification of ADEP analogs that target HsCIpP. These analogs increase both the peptidase and protease activity of HsCIpP in vitro and displace HsCIpX from HsCIpP at low compound concentrations. Importantly, treatment of immortalized and cancer cell lines with ADEPs was found to induce cell death in an HsCIpP-dependent manner. A cell line deleted of HsCIpP showed high tolerance to all ADEP analogs tested. ADEP induced cytotoxicity via activating the intrinsic, caspase-dependent apoptosis, leading to cell death. A co-crystal structure of ADEP-HsCIpP was obtained and, unexpectedly, revealed an unusual compacted CIpP conformation.

### Materials and Methods

### Bacterial Cell Cultures

All Escherichia coli strains used for DNA propagation and protein expression (see Table 1) were grown in Luria-Bertani Broth (LB; 10 g/L bio-tryptone+5 g/L yeast extract+10 g/L NaCl) supplemented with the appropriate antibiotics, unless stated otherwise. For DNA propagation, cells were grown at 37° C. with shaking. For protein expression, cells in pre-cultures were grown 16-18 hours at 37° C. with shaking. Cells in protein expression cultures were also grown at 37° C. with shaking until induction of protein expression with 1 mM IPTG. The cultures were then maintained at 37° C. for 3-5 hours or at 18° C. for 16-18 hours.

TABLE 1
Key Resources

| | | |
|---|---|---|
| REAGENT or RESOURCE | SOURCE | IDENTIFIER |

Antibodies

| | | |
|---|---|---|
| Mouse monoclonal anti-HsClpP | Abcam | RRID: AB_941071 |
| Rabbit polyclonal anti-HsClpX | Abcam | RRID: AB_11000791 |
| Mouse monoclonal anti-GAPDH | Abcam | RRID: AB_2107448 |
| Rabbit polyclonal anti-Caspase-8 | Cell Signaling | RRID: AB_10545768 |
| | Technology | |
| Rabbit polyclonal anti-Caspase-9 | Cell Signaling | RRID: AB_2068621 |
| | Technology | |
| Rabbit monoclonal anti-PUMA | Cell Signaling | RRID: AB_2064551 |
| | Technology | |
| Goat polyclonal anti-Caspase-3 | R&D Biosystems | RRID: AB_354518 |
| Mouse monoclonal anti-Mcl-1 | EMD Millipore | RRID: AB_10806351 |
| Mouse monoclonal anti-Bcl2 | EMD Millipore | RRID: AB_11210561 |
| Mouse monoclonal anti-FLAG-tag | Sigma-Aldrich | RRID: AB_262044 |
| HRP-conjugated goat anti-rabbit | BioRad | RRID: AB_11125142 |
| IgG | | |
| HRP-conjugated goat anti-mouse | BioRad | RRID: AB_11125547 |
| IgG | | |
| HRP-conjugated rabbit anti-goat | Sigma-Aldrich | RRID: AB_258242 |
| IgG | | |

Chemicals, Peptides, and Recombinant Proteins

| | | |
|---|---|---|
| ADEP-28 | (Goodreid et al., | N/A |
| | 2016) | |
| ADEP-41 | (Goodreid et al., | N/A |
| | 2016) | |
| ADEP-06 | (Goodreid et al., | N/A |
| | 2016) | |
| ADEP-02 | (Goodreid et al., | N/A |
| | 2016) | |
| ADEP-04 | (Goodreid et al., | N/A |
| | 2016) | |
| Other ADEP analogs, see Table 3 | (Goodreid et al., | N/A |
| | 2016) | |
| N-acetyl-Trp-Leu-Ala-7-amido-4- | R&D Biosystems | Cat#S-330-02M |
| methylcoumarin | | |
| Benzyloxycarbonyl-Arg-Arg-7- | Sigma-Aldrich | Cat#C5429 |
| amido-4-methylcoumarin | | |
| N-acetyl-Leu-Glu-His-Asp-7-amido- | Sigma-Aldrich | Cat#SCP0104 |
| 4-methylcoumarin | | |
| N-acetyl-Val-Glu-Thr-Asp-7-amido- | Sigma-Aldrich | Cat#SCP0139 |
| 4-methylcoumarin | | |
| Benzyloxycarbonyl-Leu-Leu-Leu-7- | Sigma-Aldrich | Cat#C0608 |
| amido-4-methylcoumarin | | |
| N-succinyl-Leu-Tyr-7-amido-4- | MP Biochemicals | Cat#03AMC117 |
| methylcoumarin | | |
| C-terminal His6-tagged Human | (Kimber et al., | N/A |
| mitochondrial ClpP (HsClpP-His₆) | 2010) | |
| Untagged human mitochondrial | This disclosure | N/A |
| ClpP (HsClpP) | | |
| Untagged human mitochondrial | This disclosure | N/A |
| ClpX (HsClpX) | | |
| Escherichia coli ClpA (EcClpA) | (Lo et al., 2001) | N/A |
| Escherichia coli ClpP (EcClpP) | (Wojtyra et al., | N/A |
| | 2003) | |
| Yeast SUMO protease | (Lee et al., 2008) | N/A |
| Casein fluorescein isothiocyanate | Sigma-Aldrich | Cat#C3777-25MG |
| from bovine milk (Casein-FITC) | | |
| Bovine milk α-casein | Sigma-Aldrich | Cat#C6780-1G |
| Creatine phosphate | Sigma-Aldrich | Cat#27920 |
| Creatine phosphokinase | Sigma-Aldrich | Cat#C3755 |
| Adenosine 5′-triphosphate disodium | Sigma-Aldrich | Cat#A26209 |
| salt hydrate (ATP) | | |
| Sulforhodamine B (SRB) | Sigma-Aldrich | Cat#230162 |

Critical Commercial Assays

| | | |
|---|---|---|
| In Situ Cell Death Detection Kit, | Roche | Cat#Roche- |
| Fluorescein | | 11684795910 |
| MitoTracker Red CMXRos | Molecular Probes | Cat#M7512 |
| Gateway LR Clonase II Enzyme Mix | Thermo-Fisher | Cat#11791020 |
| | Scientific | |
| Gateway BP Clonase II Enzyme | Thermo-Fisher | Cat#11789020 |
| Mix | Scientific | |
| Seahorse XF Cell Mitochondrial | Agilent | Cat#103015-100 |
| Stress Kit | | |

Deposited Data

| | | |
|---|---|---|
| X-ray structure: HsClpP in complex | This disclosure | PDB: 6BBA |
| with ADEP-28 | | |
| X-ray structure: HsClpP | (Kang et al., 2004) | PDB: 1TG6 |
| X-ray structure: BsClpP in complex | (Lee et al., 2010) | PDB: 3KTI |
| with ADEP1 | | |
| X-ray structure: EcClpP | (Bewley et al., | PDB: 1YG6 |
| | 2006) | |
| X-ray structure: EcClpP A153C | (Kimber et al., | PDB: 3HLN |
| | 2010) | |
| X-ray structure: EcClpP in complex | (Li et al., 2010) | PDB: 3MT6 |
| with ADEP1 | | |
| X-ray structure: HpClpP in complex | (Kim and Kim, | PDB: 2ZL2 |
| with peptide NVLGFTQ | 2008) | |
| X-ray structure: LmClpP1 N65D | (Zeiler et al., 2013) | PDB: 4JCR |
| X-ray structure: LmClpP1 | (Zeiler et al., 2013) | PDB: 4JCQ |
| X-ray structure: MtClpP1 | (Ingvarsson et al., | PDB: 2CE3 |
| | 2007) | |
| X-ray structure: MtClpP1P2 in | (Schmitz et al., | PDB: 4U0G |
| complex with ADEP | 2014) | |
| X-ray structure: NmClpP in complex | (Goodreid et al., | PDB: 5DKP |
| with ADEP A54556 | 2016) | |
| X-ray structure: PfClpP | (El Bakkouri et al., | PDB: 2F6I |
| | 2010) | |
| X-ray structure: SaClpP (extended) | (Zhang et al., 2011) | PDB: 3STA |
| X-ray structure: SaClpP (compact) | (Ye et al., 2013) | PDB: 4EMM |
| X-ray structure: SaClpP | (Zhang et al., 2011) | PDB: 3ST9 |
| (compressed) | | |
| X-ray structure: SaClpP in complex | This disclosure | PDB: 5VZ2 |
| with ADEP | | |
| X-ray structure: SpClpP A153P | (Gribun et al., | PDB: 1Y7O |
| | 2005) | |

Experimental Models: Cell Lines

| | | |
|---|---|---|
| HEK293 T-REx | Gift from A. | RRID: CVCL_U427 |
| | Trifunovic | |
| HEK293 T-REx CLPP^{-/-} | (Seiferling et al., | N/A |
| | 2016) | |
| HEK293 T-REx + CLPP-FLAG | This disclosure | N/A |
| HEK293 T-REx + CLPP_{S153A}-FLAG | This disclosure | N/A |
| HEK293 T-REx ΔCLPX* | This disclosure | N/A |
| HEK293 T-REx CLPP^{-/-} ΔCLPX* | This disclosure | N/A |
| HeLa | Gift from P. Kim | RRID: CVCL_0030 |
| HeLa T-REx | Gift from L. | RRID: CVCL_D587 |
| | Attisano | |
| U2OS | Gift from A. | RRID: CVCL_0042 |
| | Palazzo | |
| SH-SY5Y | Gift from M. Babu | RRID: CVCL_0019 |

Experimental Models: Organisms/Strains

| | | |
|---|---|---|
| Escherichia coli DH5α | Lab stock | NCBI: txid668369 |
| Escherichia coli BL21(DE3) | (Kimber et al., | N/A |
| ΔclpP::cat (SG1146) | 2010) | |

Oligonucleotides

| | | |
|---|---|---|
| CLPX Exon1 pX330 F: | This disclosure | N/A |
| CACCGCGGTGCTTGTACTTGCG | | |
| GCG (SEQ ID NO: 1) | | |
| CLPX Exon1 pX330 R: | This disclosure | N/A |
| AAACCGCCGCAAGTACAAGCAC | | |
| CGC (SEQ ID NO: 2) | | |
| CLPX Exon1 ssODN: | This disclosure | N/A |
| GGCCTCGCGGAGATGCCCAGCT | | |
| GCGGTGCTTGTACTTGCGGCGC | | |
| GGCGTAGGTCCGGCTCATCACC | | |
| TCCTCACTCGCCTCCGCGCAGA | | |
| GA (SEQ ID NO: 3) | | |

Recombinant DNA

| | | |
|---|---|---|
| pDT1668-LhclpP | (Kimber et al., | N/A |
| | 2010) | |
| pET9a-EcClpA M168T | (Lo et al., 2001) | N/A |
| pET9a-EcClpP | (Wojtyra et al., | N/A |
| | 2003) | |
| pET28-Ulp1 | (Lee et al., 2008) | N/A |
| pETSUMO | (Lee et al., 2008) | N/A |
| pETSUMO2-CLPP(-MTS) | This disclosure | N/A |
| pST39 | (Selleck and Tan, | N/A |
| | 2008) | |
| pST39-SUMO2-CLPX(-MTS) | This disclosure | N/A |
| pDEST-MTS-HsCLPP-3xFLAG | This disclosure | N/A |
| pDEST-MTS-HsCLPP_{S153A}-3xFLAG | This disclosure | N/A |
| pOG44 | Thermo-Fisher | Cat#V600520 |
| | Scientific | |
| pX330 | (Cong et al., 2013) | N/A |
| pX330-CLPX KO | This disclosure | N/A |

Software and Algorithms

| | | |
|---|---|---|
| HKL2000 | (Otwinowski and | http://www.hkl- |
| | Minor, 1997) | xray.com/download- |
| | | instructions-hkl-2000 |
| PHENIX | (Adams et al., | https://www.phenix- |
| | 2010) | online.org/ |
| COOT | (Emsley and | http://www2.mrc- |
| | Cowtan, 2004) | lmb.cam.ac.uk/Personal/ |
| | | pemsley/coot/ |
| ATSAS 2.7.2 software package | (Petoukhov et al., | https://www.embl- |
| | 2012) | hamburg.de/biosaxs/ |
| | | manuals/atsas- |
| | | 2.7/install.html |
| UCSF Chimera software | (Pettersen et al., | https://www.cgl.ucsf.edu/ |
| | 2004) | chimera/ |
| MaxQuant software 1.6.1.0 | Max Planck | http://www.coxdocs.org/ |
| | Institute | doku.php?id=maxquant: |
| | | common:download_and_ |
| | | installation#download_ |
| | | and_installation_guide |
| NIS-Elements Basic Research | Nikon | https://www.nikoninstruments.com/ |
| software | | Products/Software/NIS- |
| | | Elements-Basic-Research |
| ZEN Black software 8.1 | Carl Zeiss | https://www.zeiss.com/ |
| | Microscopy | microscopy/int/software- |
| | | cameras.html |
| Wave Desktop software | Agilent | https://www.agilent.com/ |
| | | en/products/cell- |
| | | analysis- |
| | | (seahorse)/software- |
| | | download-for-wave- |
| | | desktop |
| Quantity One 1D Analysis software | BioRad | http://www.bio- |
| | | rad.com/en- |
| | | ca/product/quantity-one- |
| | | 1-d-analysis- |
| | | software?ID=1de9eb3a- |
| | | 1eb5-4edb-82d2- |
| | | 68b91bf360fb |

Other

| | | |
|---|---|---|
| Rigaku FR-E SuperBright rotating | Structural | https://www.thesgc.org/ |
| anode generator | Genomics | science/crystallography |
| | Consortium, | |
| | University of | |
| | Toronto, Toronto, | |
| | Canada | |
| Pilatus 300K detector, SAXS1 | Brazilian | http://www.lnls.cnpem.br/ |
| beamline | Synchrotron Light | linhas-de-luz/saxs1- |
| | Laboratory, LNLS, | en/overview/ |
| | CNPEM, | |
| | Campinas, Brazil | |
| Proxeon EASY nanoLC 1000 | Thermo-Fisher | Cat#LC120 |
| System | Scientific | |
| Orbitrap Elite mass spectrometer | Thermo-Fisher | Cat#IQLAAEGAAPFAD |
| | Scientific | BMAZQ |
| Acclaim PepMap C18 column (15 | Thermo-Fisher | https://www.thermofisher.com/ |
| cm x 50 μm ID, 3 μm, 100 Å) | Scientific | order/catalog/product/ |
| | | 160321 |
| Eclipse 80i fluorescence | Nikon | https://www.nikoninstruments.com/ |
| microscope | | Products/Upright- |
| | | Microscopes/Research/ |
| | | Eclipse-80i |
| X-Cite Series 120Q excitation | Excelitas | http://www.excelitas.com/ |
| light source | Technologies | Pages/Product/X-Cite- |
| | | 120Q.aspx |
| ELYRA PS.1 superresolution | Carl Zeiss | https://www.zeiss.com/ |
| microscope | Microscopy | microscopy/int/products/ |
| | | superresolution- |
| | | microscopy.html |
| iXon 885 EMCCD camera | Andor Technology | http://www.andor.com/ |
| | | cameras/ixon-emccd- |
| | | camera-series |
| Microscope coverslips for SIM | Electron | Cat#72230-01 |
| | Microscopy | |
| | Sciences | |
| Microscope glass slides for SIM | VWR | Cat#48312-401 |
| ChemiDoc XRS + System | BioRad | http://www.bio- |
| | | rad.com/en- |
| | | ca/product/chemidoc- |
| | | xrs- |
| | | system?ID=NINJHRKG4 |
| Seahorse XFe96 Metabolic Flux | Agilent | https://www.agilent.com/ |
| Analyzer | | en/products/cell- |
| | | analysis- |
| | | (seahorse)/seahorse- |
| | | analyzers/seahorse- |
| | | xfe96-analyzer |
| Seahorse XFe96 Sensor Cartridge | Agilent | Cat#102416-100 |
| Seahorse XF96 Cell Culture | Agilent | Cat#101085-004 |
| Microplates | | |
| EnSpire 2300 Multilabel Reader | Perkin-Elmer | http://www.perkinelmer.com/ |
| | | product/enspire- |
| | | base-unit-2300-0000 |
| 96-well black flat-bottom plate | Greiner Bio-one | Cat#655077 |
| | International | |

### Mammalian Cell Cultures

All mammalian cell lines used (see Table 1) were maintained in Dulbecco's modified Eagle's medium (DMEM) supplemented with 10% fetal bovine serum (FBS), 2 mM L-glutamine and 100 U/mL penicillin-streptomycin, unless stated otherwise. Cells were grown and maintained in standard tissue culture plates or tissue culture flasks with ventilated caps. For DNA transfection, cells were kept in the same media but without any antibiotics at least 24 hours prior to the procedure. All cells were passaged by standard trypsinization procedures at least three times before use in any experiments.

For long-term cryogenic storage, cells were first trypsinized and re-suspended in media, followed by gradual addition of 80% FBS+20% DMSO to a final 1:1 (v:v) ratio. The cell stocks were then transferred into cryogenic storage vials and stored in foam storage racks at −80° C. for at least 24 hours before long-term storage in a liquid nitrogen storage tank. To recover cells from the frozen stocks, the frozen cells were thawed at 37° C. and re-suspended in media at 9× the frozen stock's volume. The media was applied gradually to the cells to minimize osmotic shock. Cells were then collected by centrifugation and re-suspended in fresh media after which, they were transferred to tissue culture plates or ventilated tissue culture flasks for growth and maintenance as described above.

### Molecular Cloning and Protein Purification

For protein expression, CLPP gene was cloned without its mitochondrial targeting sequence (MTS) (residues M1-P57) into a modified pETSUMO vector (Lee et al., 2008) resulting in pETSUMO2-CLPP(-MTS) expressing 2×(His6-thrombin)-SUMO-CLPP(-MTS). CLPX gene was also cloned without its MTS (residues M1-F64) into a modified pST39 (Selleck and Tan, 2008) resulting in pST39-SUMO2-CLPX(-MTS) expressing 2x(His6-thrombin)-SUMO-CLPX(-MTS). All plasmids were propagated in E. coli DH5α and isolated using the PureLink Quick Plasmid Miniprep Kit (Thermo-Fisher Scientific). Linearized DNA and PCR amplification products were purified by gel extraction using the PureLink Quick Gel Extraction Kit (Thermo-Fisher Scientific).

HsCIpP was expressed with a N-terminal 2×(His6-thrombin)-SUMO tag in E. coli SG1146, which is BL21(DE3) ΔcIpP::cat (Kimber et al., 2010). Cells transformed with pETSUMO2-CLPP(-MTS) were grown aerobically in LB+50 μg/mL kanamycin at 37° C. until OD₆₀₀ reached ˜0.6. Protein expression was then induced with 1.5 mM IPTG (Thermo-Fisher Scientific) for 3-5 hours. Cells were harvested by centrifugation, and then re-suspended in 25 mM TrisHCl, pH 7.5, 0.5 M NaCl, 10% glycerol, and 10 mM imidazole. Cells were then lysed by 2 passes on the French Press, and the cell debris was removed by centrifugation. The SUMO-tagged HsCIpP was purified on Ni-NTA beads (Thermo-Fisher Scientific) using standard protocols. Subsequently, SUMO protease (Lee et al., 2008) was added to remove the N-terminal 2×(His6-thrombin)-SUMO tag. The purified, untagged HsCIpP was concentrated with an Amicon Ultra-15 centrifugal filter unit (10000 MWCO) (EMD Millipore) at 4° C., aliquoted, flash-frozen in liquid nitrogen and stored at −80° C. until use. A similar protocol was used for HsCIpX. All fractions collected during the purification were analyzed by SDS-PAGE. The proteins were found to be >95% pure.

For high-throughput drug activity assays, C-terminally Hiss-tagged HsCIpP (HsCIpP-Hiss) was expressed in SG1146 transformed with pDT1668-LhclpP and purified as described in (Kimber et al., 2010). Untagged E. coli CIpP (EcCIpP) was expressed and purified as described in (Wojtyra et al., 2003). E. coli CIpA (EcCIpA) was expressed and purified as described in (Lo et al., 2001).

### Screen of ADEP Analogs Against HsCIpP

Structural analogs of acyldepsipeptide (ADEP) (Goodreid et al., 2016) that were previously developed as potential antibiotics against bacterial CIpP were screened and assessed for their ability to activate and dysregulate the protease activity of HsCIpP, by measuring their relative degradation (RD) index. Note that ADEP-28 was also previously reported by Carney et al. (2014). Details of the experimental protocol have been described in Leung et al., 2011. The RD index is defined as follows:

$$RD = \frac{{( Δ {ϕ}_{ClpP + compound} )}_{after 6 hrs} - {( Δ {ϕ}_{ClpP} )}_{after 6 hrs}}{{( Δ {ϕ}_{E . coli ClpAP} )}_{after 6 hrs} - {( Δ {ϕ}_{E . coli ClpP} )}_{after 6 hrs}} .$$

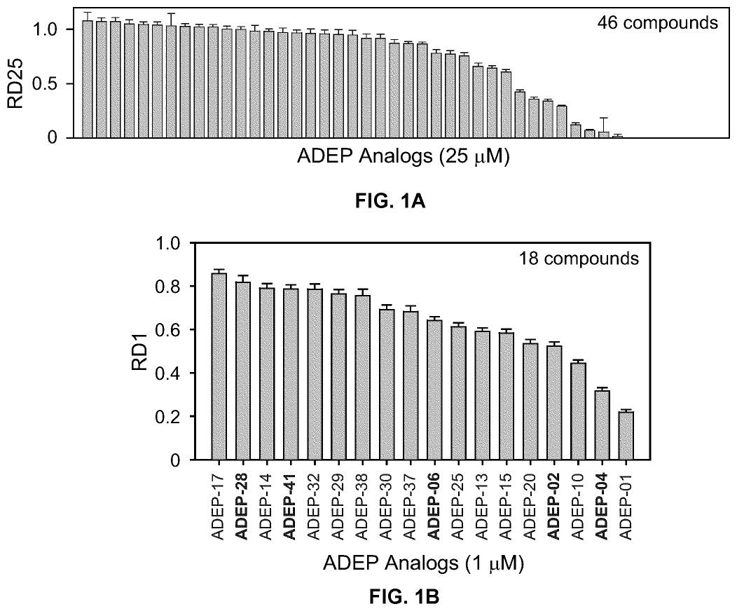

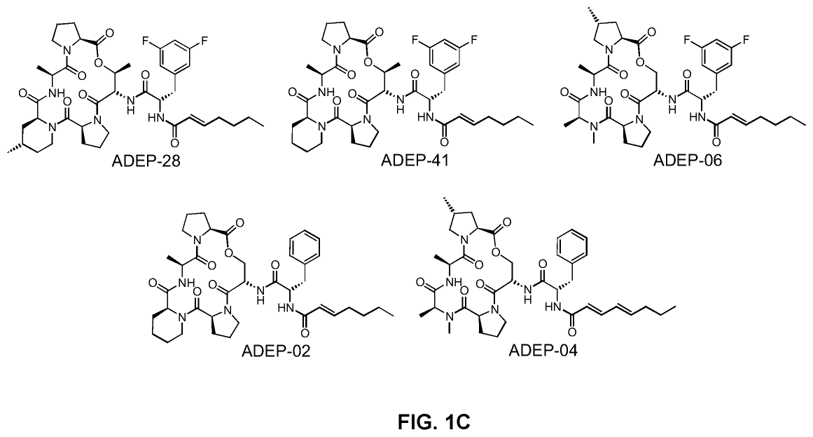

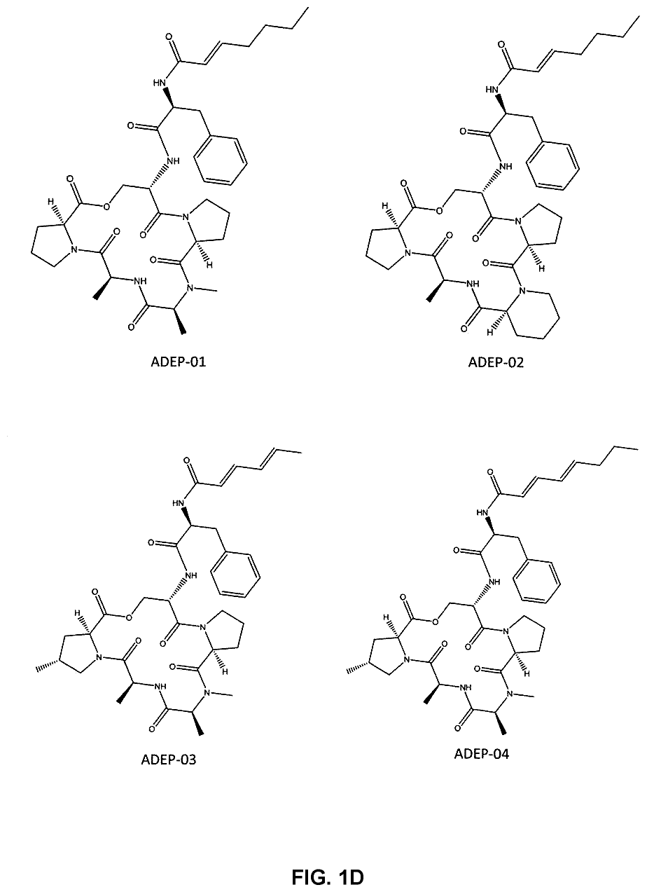

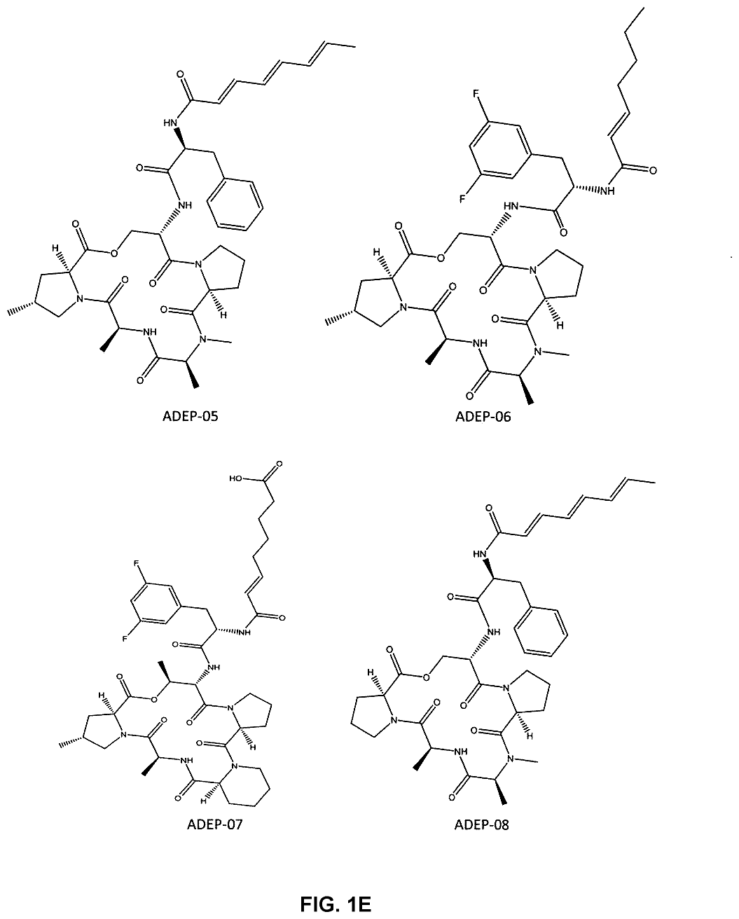

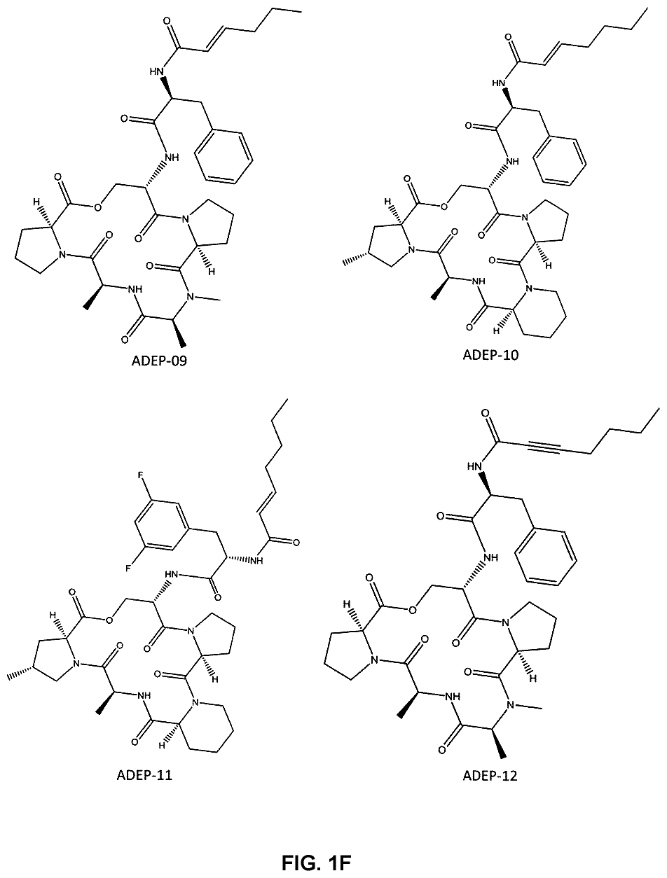

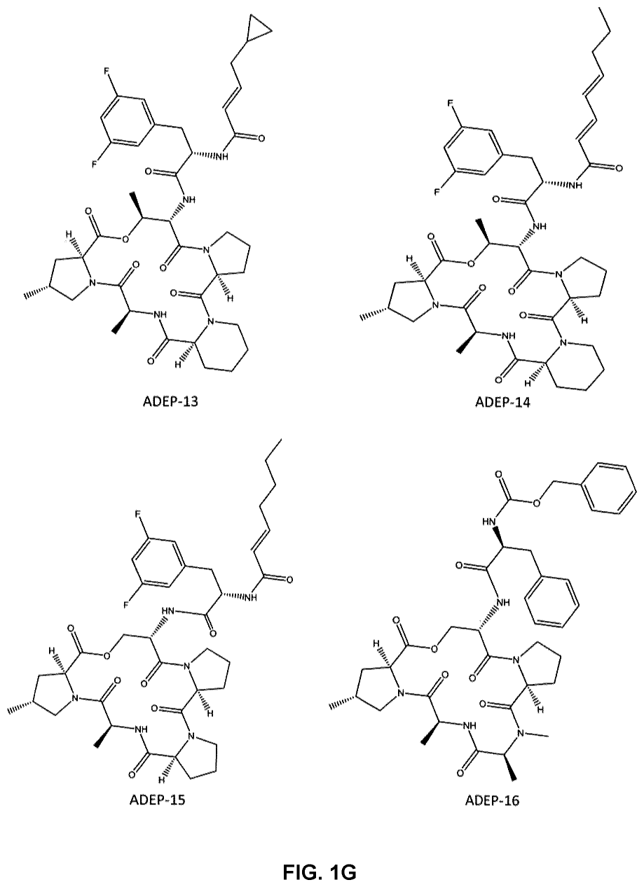

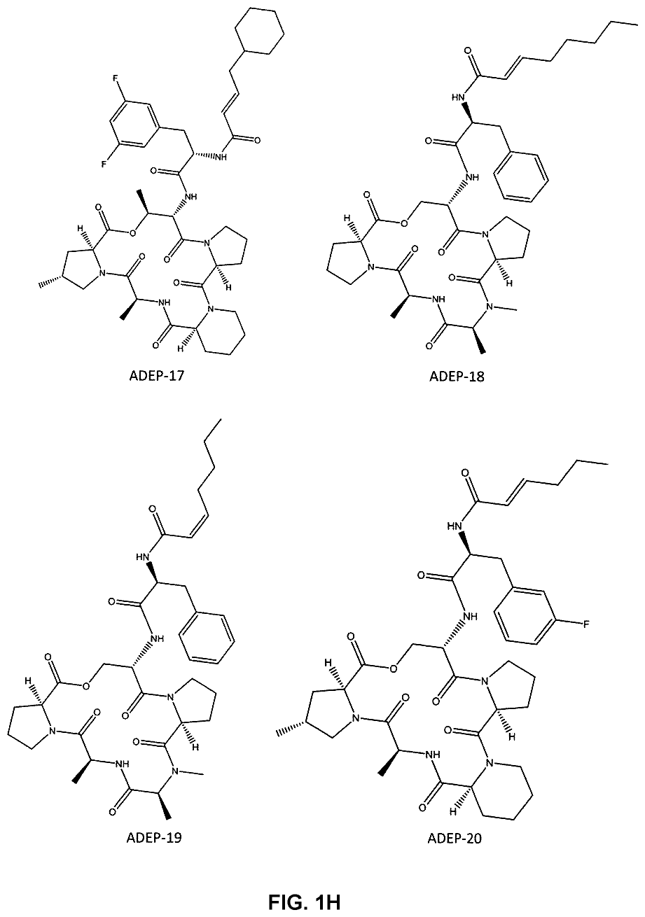

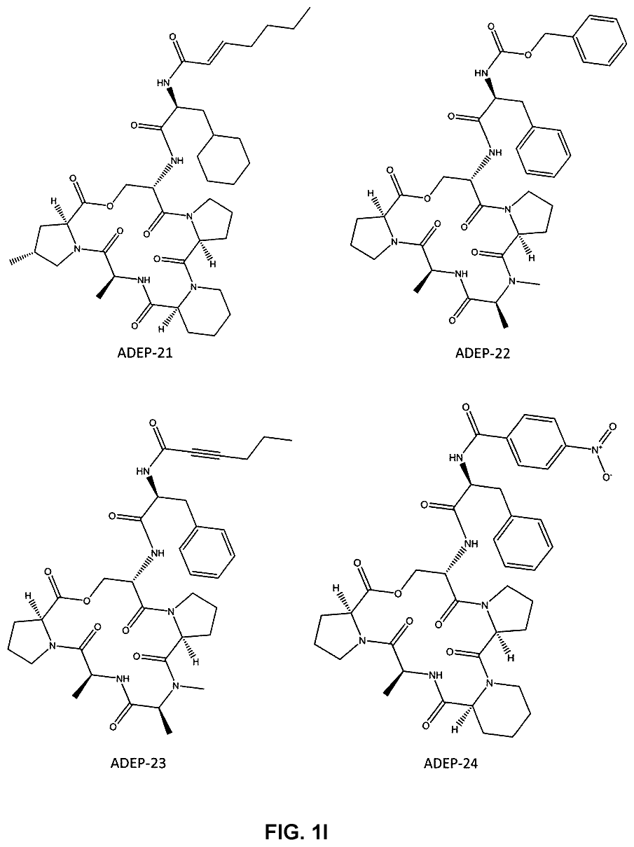

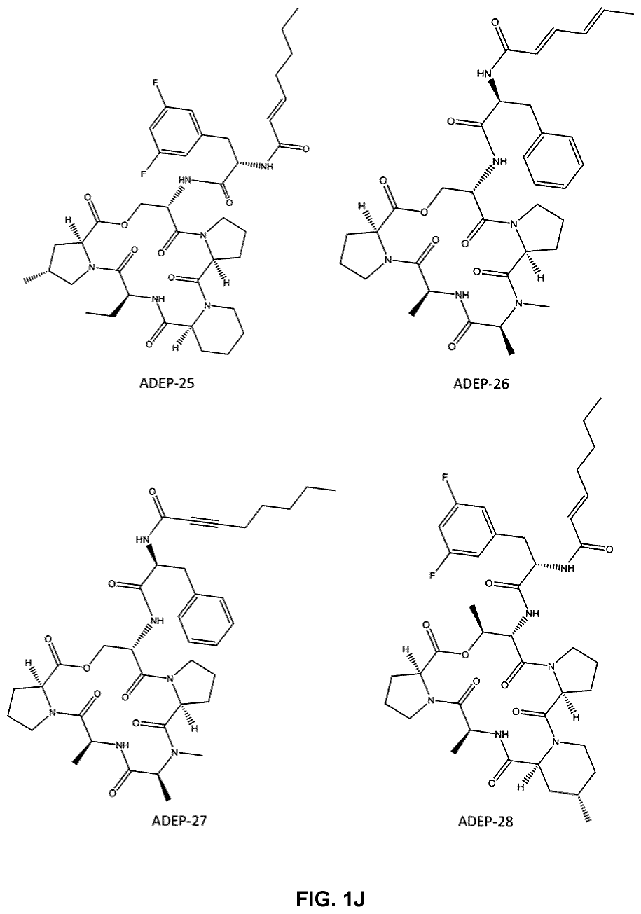

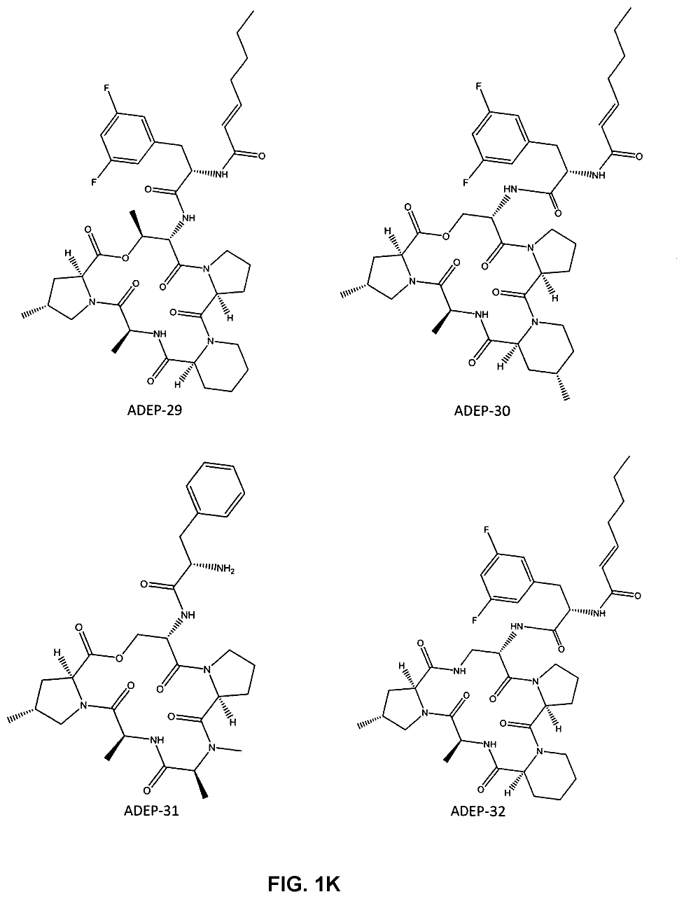

Δφ is the change in fluorescence after 6 hr of starting the reaction measured using 485 nm excitation and 535 nm emission, which primarily detects the signal from casein-FITC. E. coli CIpAP was used as a benchmark for maximum CIpP proteolytic activity. The CIpP in the numerator can be from any other organism. RD25 measurements refer to the measurement in the presence of 25 μM compound. All ADEP analogs were first screened at 25 μM, and their RD25 scores were calculated using the above formula and method as described in Leung et al., 2011 (FIG. 1A). Those that achieved RD25≥0.87 were screened a second time at 5 μM, followed by calculation of their RD5 scores. The top analogs with RD5≥0.74 were screened a third time at 1 μM, followed by calculation of their RD1 scores (FIG. 1B).

### Peptidase Activity Assays

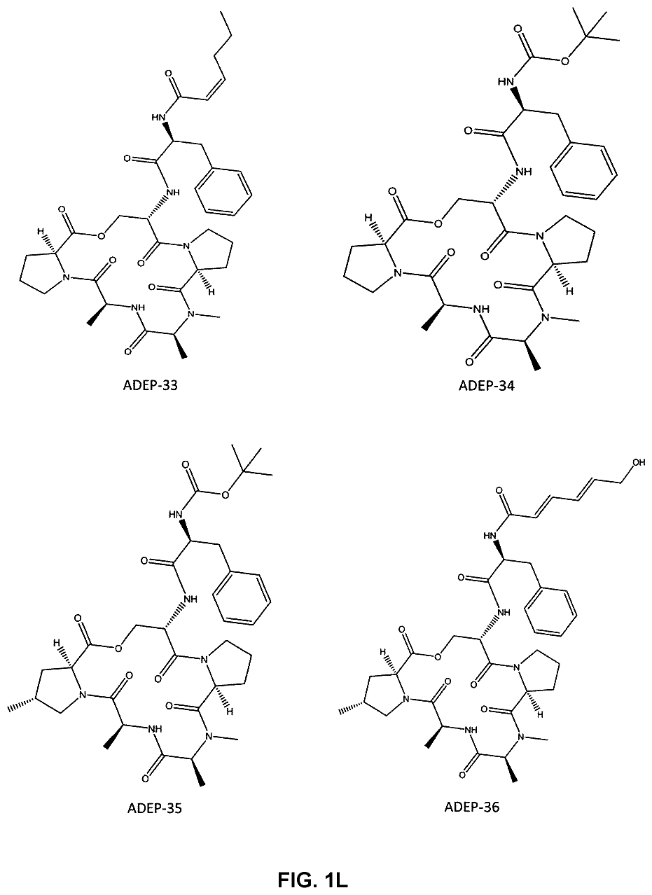

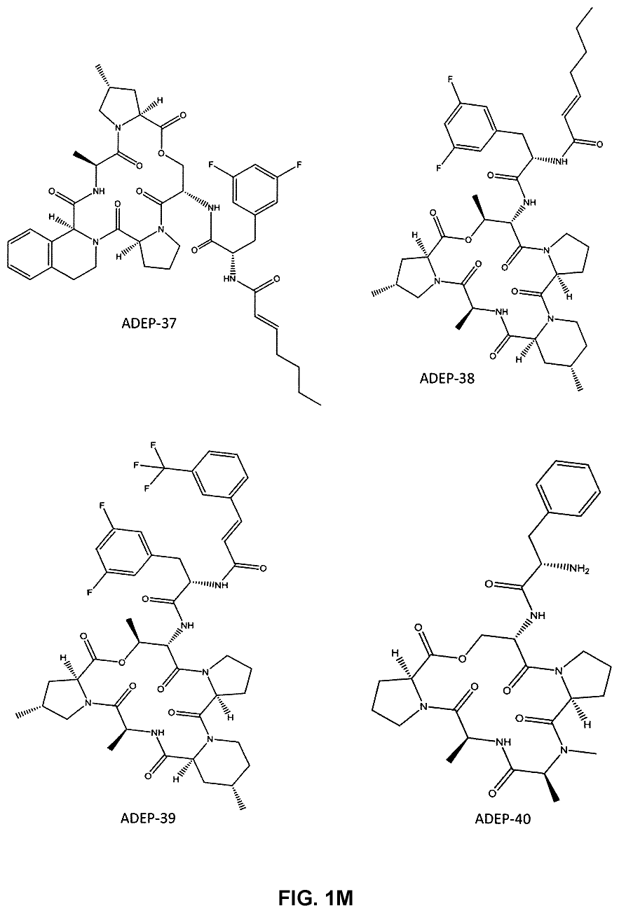

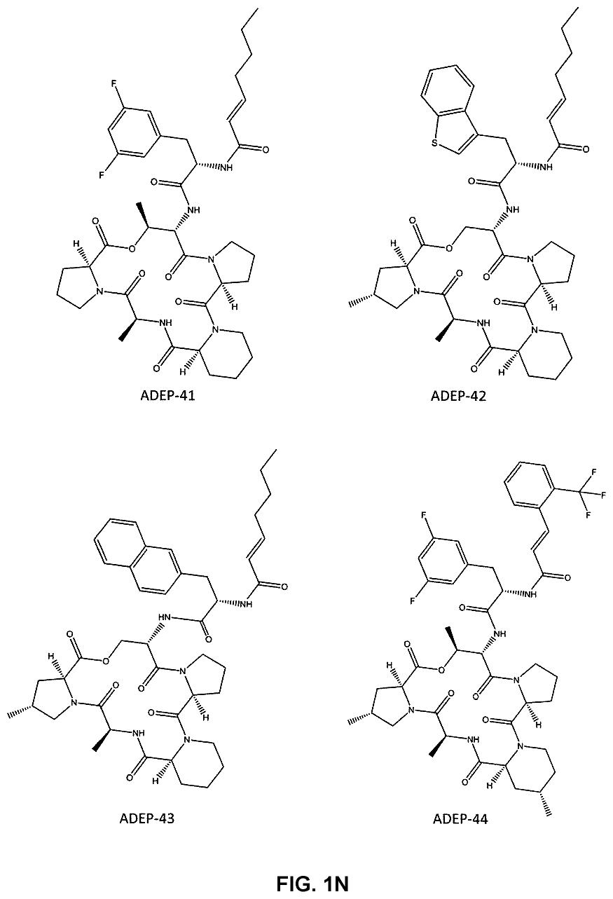

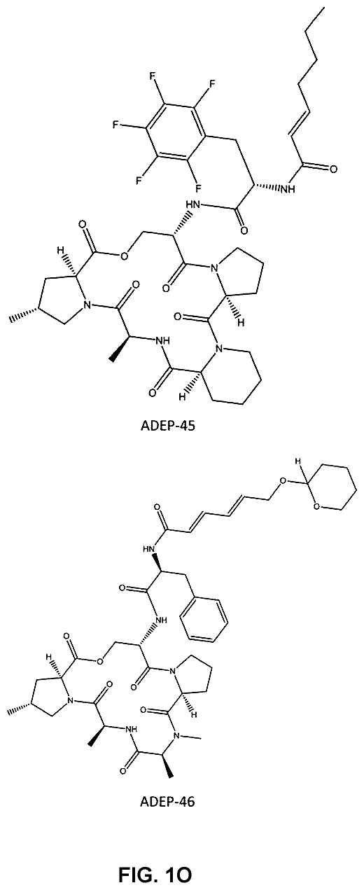

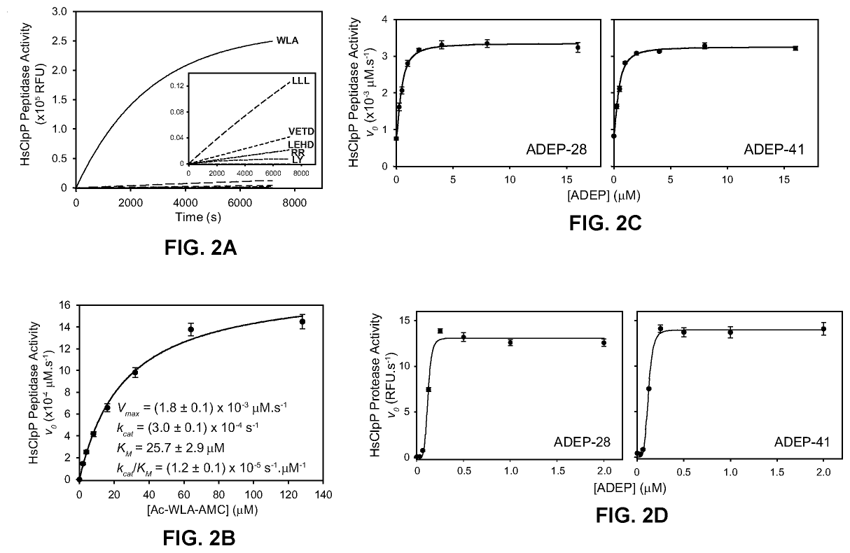

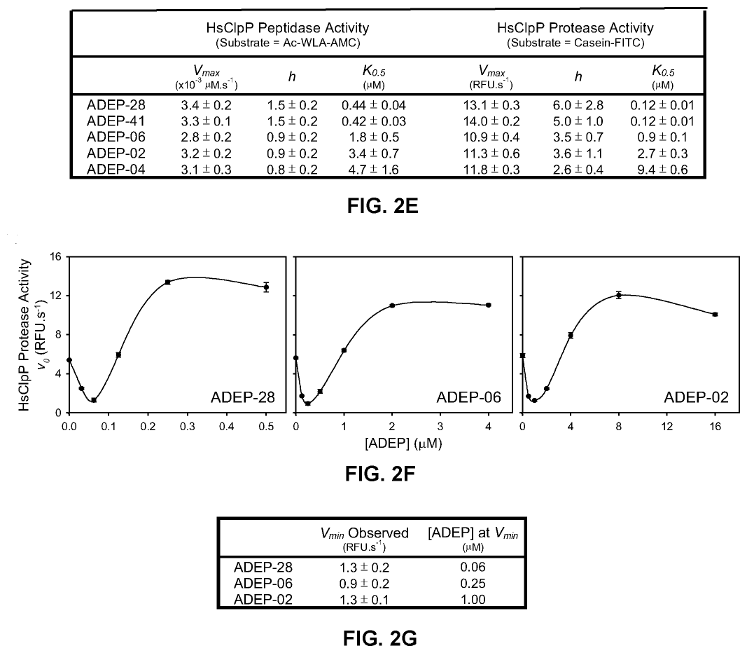

The peptidase activity of purified, untagged HsCIpP was assessed by monitoring the degradation of peptidyl substrates labelled with a C-terminal 7-amido-4-methylcoumarin (AMC) fluorophore. As previously reported (Kang et al., 2002), HsCIpP did not exhibit any observable peptidase activity towards the commonly used N-succinyl-Leu-Tyr-7-amido-4-methylcoumarin (Suc-LY-AMC) peptide (FIG. 2A). To identify a suitable peptidyl substrate, commercially available AMC-labelled peptides were purchased and tested for degradation by HsCIpP. These include N-acetyl-Trp-Leu-Ala-AMC (Ac-WLA-AMC) (R&D Systems) that was also previously used by Csizmadia et (Csizmadia et al., 2016), benzyloxycarbonyl-Arg-Arg-AMC (Z-RR-AMC) (Sigma-Aldrich), N-acetyl-Leu-Glu-His-Asp-AMC (Ac-LEND-AMC) (Sigma-Aldrich), N-acetyl-Val-Glu-Thr-Asp-AMC (Ac-VETD-AMC) (Sigma-Aldrich), and benzyloxycarbonyl-Leu-Leu-Leu-AMC (Z-LLL-AMC) (Sigma-Aldrich). Suc-LY-AMC (MP Biomedicals) was also tested as a negative control. All stock solutions of the AMC-labelled peptides were prepared by dissolving the lyophilized peptides in DMSO at 90 mM or a lower concentration recommended by the manufacturer. All assays were performed in 50 mM TrisHCl, pH 8, 10 mM MgCl₂, 100 mM KCl, 0.02% Triton X-100 (v:v), 5% glycerol (v:v), and 1 mM DTT. HsCIpP was used at 8 μM (final monomeric concentration) for the initial screening for a suitable substrate, but at 6 μM in subsequent experiments. All AMC-labelled peptides were used at the final concentration of 500 μM in the initial screen. Ac-WLA-AMC was used at the final concentration of 100 μM in all other experiments. Addition of the peptides also raised the final concentration of DMSO in the reaction mix to 3% (v:v). All reactions were carried out with 150 μL of the reaction mix per well on a 96-well black flat-bottom plate (Greiner) at 37° C. Degradation of the peptidyl substrates and subsequent release of the free AMC moiety was monitored by fluorescence with λₑₓ=360 nm and λₑₘ=440 nm using the EnSpire 2300 Multilabel Reader (Perkin-Elmer) set to perform a reading every 30 s with 20 flashes. A total of 240 readings were collected for each of three independent replicates prepared for each peptidyl substrate.

### Protease Activity Assays

The protease activity of purified HsCIpP was assessed by monitoring the degradation of bovine milk casein labelled with fluorescein isothiocyanate (casein-FITC) (Sigma-Aldrich) in the presence of ADEP and/or purified HsCIpX. All reactions were carried out in 25 mM HEPES, pH 7.5, 35 mM KCl, 25 mM MgCl₂, 0.03% Tween-20, 10% glycerol, and 1 mM DTT, supplemented with 16 mM creatine phosphate and 300 μM ATP. HsCIpP was used at 3 μM (final monomeric concentration) and casein-FITC was added at the final concentration of 10 μM. 13 U of creatine phosphokinase (Sigma-Aldrich) was also added for ATP regeneration. DMSO was present at 1% (v:v) final concentration in the experiments that involve the use of ADEP analogs. All reactions were carried out with 150 μL of the reaction mix per well in a 96-well black flat-bottom plate at 37° C. Degradation of casein-FITC and subsequent release of the free FITC moiety was monitored by fluorescence with λₑₓ=494 nm and λₑₘ=518 nm using the EnSpire 2300 Multilabel Reader set to perform a reading every 40 s with 20 flashes. A total of 100 readings were collected in each experiment.

To investigate the casein-FITC degradation by HsCIpXP, kinetic analysis was performed with HsCIpP held constant at 3 μM (final monomeric concentration) and HsCIpX was titrated from 0 to 18 μM (final monomeric concentration). Initial rates (v₀) were determined from data collected over three independent experiments. Non-linear regression analysis was then performed on the initial rates data using the Hill equation, as follows:

$${v}_{0} = \frac{{{V}_{max} [ HsClpX or ADEP ]}^{h}}{{K}_{0.5}^{h} + {[ HsClpX or ADEP ]}^{h}}$$

Vₘₐₓ is maximal velocity, h is the Hill coefficient, and K_{0.5} is the microscopic apparent dissociation constant.

The effect of ADEP in activating HsCIpP was assessed via the degradation of casein-FITC by HsCIpP independent of HsCIpX. All reactions were carried out with HsCIpP at 3 μM (final monomeric concentration) in the same way as described above. ADEP analogs were titrated from 0-2 μM or 0-128 μM, depending on their potency. Initial rates data were collected and analyzed using the Hill equation above to determine Vₘₐₓ, h and K_{0.5} for each ADEP analog. For experiments that require HsCIpX in the reaction, the protein was added at 9 μM (final monomeric concentration).

For gel-based degradation assays, unlabelled bovine milk α-casein (Sigma-Aldrich; C6780-1G) was used in place of casein-FITC in the same reaction condition as before. HsCIpP and HsCIpX were used at 6 μM and 18 μM (both final monomeric concentrations), respectively, to ensure that sufficient amount of casein was degraded to be observable by SDS-PAGE, while maintaining the same HsCIpP-to-HsCIpX ratio as previously used. ATP concentration was also raised to 3 mM. As a representative analog, ADEP-28 was used at 0.1 μM and 2 μM alongside a DMSO-only control. Samples were collected at designated time points and analyzed by SDS-PAGE. Casein degradation was quantified by densitometry using the Quantity One 1D Analysis Software (BioRad). Three independent experiments were performed for each concentration of ADEP-28 and the DMSO-only control.

### X-Ray Structure Determination of ADEP-28-HsCIpP

Purified untagged HsCIpP was dialyzed against buffer containing 25 mM Bis-Tris, pH 6.5 and 3 mM dithiothreitol, concentrated to 10 mg/mL, and 750 μM of ADEP-28 was added. The solution was pre-incubated for 1 hr at 37° C. Subsequently, 0.5 μL of the protein-ADEP-28 solution was mixed with 0.5 μL of crystallization reagent (0.1 M sodium acetate trihydrate pH 4.6, 4% w/v Polyethylene glycol 4,000). The drops were equilibrated against the crystallization reagent in the reservoir at 21° C. Large crystals of HsCIpP were observed within two weeks and grew to maximal dimensions within a month.

Crystals of HsCIpP were cryoprotected by quick soaking in crystallization solution containing 20% glycerol and 1 mM ADEP-28, then flash-cooled in liquid nitrogen. Diffraction data were collected at the Structural Genomics Consortium-University of Toronto at 100 K and wavelength of 1.5418 Å using Rigaku FR-E SuperBright rotating anode generator. Diffraction data were indexed, integrated, and scaled in HKL2000 (Otwinowski and Minor, 1997). The structure of HsCIpP complex with ADEP-28 was determined by molecular replacement using Phaser in PHENIX (Adams et al., 2010) with the published apo-HsCIpP structure (PDB 1TG6) (Kang et al., 2004) as search model. The crystal asymmetric unit contained 7 subunits of HsCIpP forming a single heptameric ring. The structure was refined initially in PHENIX with simulated annealing and coordinate shaking to remove model bias. Subsequent model building and refinement were performed in COOT (Emsley and Cowtan, 2004) and PHENIX (Adams et al., 2010) using translation-libration-screw (TLS) parameters with individual coordinate, occupancy and B factor optimization. The final model was refined to an R_{work}/R_{free} of 0.19/0.24 up to a resolution of 2.80 Å. The model has good geometry with >97% of amino acid residues in favored and allowed regions of the Ramachandran plot. Data collection and refinement statistics are summarized in Table 2. The PDB accession number is 6BBA.

TABLE 2
Data collection and refinement statistics of
ADEP-28-HsCIpP structure Data Collection
Data Collection

| | |
|---|---|
| Space group | P 3₂ 2 1 |

Cell dimensions

| a, b, c (Å) | 172.40, 172.40, 135.96 | |
|---|---|---|
| α, β, γ (°) | 90.0, 90.0, 120.0, | |
| Wavelength (Å) | 1.5418 | |
| Resolution (Å) | 46.73-2.80 | (2.90-2.80) |
| Rₘₑₐₛ/Rₚᵢₘ | 0.188/0.056 | |
| \|/σ\| | 15.4 | (1.3) |
| Completeness (%) | 99.9 | (100.0) |
| Redundancy | 11.1 | (11.0) |

Refinement

| Resolution (Å) | 46.73-2.80 | |
|---|---|---|
| No. reflections | 57,813 | (5,716) |

| | |
|---|---|
| R_{work}/R_{free} | 0.1866/0.2346 |
| No. atoms | 10,846 |
| Protein | 10,246 |
| Ligand | 392 |
| Water | 208 |

B factors

| | |
|---|---|
| Protein | 62.9 |
| Ligand | 61.3 |
| Water | 57.7 |

r.m.s. deviations

| | |
|---|---|
| Bond lengths (Å) | 0.010 |
| Bond angles (°) | 1.18 |

Values in parentheses are for the highest resolution shell.
The highest resolution shell includes all reflections between 2.80 and 2.90 Å.
R_{work} is Σ|Fₒ-F_{c}|/ΣFₒ.
R_{free} is the cross-validation R-factor computed for a test set of reflections (3.5% of total).

### Small Angle X-Ray Scattering (SAXS) Experiments

Data collection was performed using a Pilatus 300K detector in the SAXS1 beamline, located at the Brazilian Synchrotron Light Laboratory (LNLS, CNPEM, Campinas, Brazil). Measurements were done using a monochromatic X-ray beam (λ=1.488 Å) and sample-to-detector distance of ˜1,000 mm. The scattering intensity, I(q), was detected as a function of the scattering vector (q), where q=4π sinθ/λ and 2θ is the scattering angle. The range for q was between 0.013 Å⁻¹ and 0.5 Å⁻¹. Samples were prepared at 0.5 mg/mL in buffer of 50 mM TrisHCl, pH 7.5, 200 mM KCl, 25 mM MgCl₂ and 10% glycerol. The final concentration of DMSO was kept at 2.3% in samples containing ADEP (0.6 mM final) or DMSO only. Ten frames of 10 seconds and one frame of 300 seconds were recorded for every sample at 20° C. to inspect for X-ray damage.

Data analysis was performed using software from the ATSAS 2.7.2 package (Petoukhov et al., 2012). All data curves were verified for X-ray damage and aggregation using the Guinier approximation and the PRIMUS software. Molecular weight determination was performed by comparing the forward scattering intensity 1(0)-values obtained from Guinier approximation and I(0)-values determined from standard BSA samples (Barbosa et al., 2013; Mylonas and Svergun, 2007). Curves were normalized by protein concentration. The pair distance distribution functions, p(r), were generated by the GNOM software. Generation of dummy atoms models (DAMs) was performed using the SAXS curve until q=0.2 and P72 symmetry in the simulated-annealing method implemented by the DAMMIF software. Ten DAM models were averaged using the DAMAVER package. Superimposition of crystallographic structures and DAMs was performed using the SUPCOMB program. Visualization of DAMs and generation of density maps were done using the UCSF Chimera software (Pettersen et al., 2004).

### Testing the Effect of ADEPs on Different Cell Lines

Different cell lines were grown and maintained in Dulbecco's

Modified Eagle Media (DMEM) (Gibco) supplemented with 10% fetal bovine serum (Gibco), 100 U/mL penicillin-streptomycin (Gibco) and 2 mM L-glutamine (Gibco) at 37° C. under a moist atmosphere with 6% CO₂. All cells were passaged at least 3 times prior to use.

Cell lines used in the experiments presented here were acquired from various sources. HEK293 T-REx wild-type (WT) and HEK293 T-REx CLPP^{-/-} cells were obtained from Professor Aleksandra Trifunovic (University of Cologne, Germany). HeLa (regular) cells were obtained from Professor Peter Kim (Hospital for Sick Children, Canada). HeLa T-REx cells were obtained from Professor Lilianna Attisano (University of Toronto, Canada). U2OS cells were obtained from Professor Alex Palazzo (University of Toronto, Canada). Undifferentiated SH-SYSY cells were obtained from Professor Mohan Babu (University of Regina, Canada).

HEK293 T-REx+CLPP-FLAG and HEK293 T-REx+CLPP_{S153A}-FLAG were generated using the Flp-In System (Sigma-Aldrich). Briefly, full length CLPP gene that includes its native MTS was cloned as pDEST-MTS-HsCLPP-3×FLAG using the Gateway system (Thermo Fisher Scientific). For expressing the proteolytically inactive HsCIpP, the S153A mutation was introduced into pDEST-MTS-HsCLPP-333 FLAG by QuikChange site-directed mutagenesis (Qiagen) to generate the pDEST-MTS-HsCLPP_{S153A}-3×FLAG. To generate the required cell lines expressing the FLAG-tagged HsCIpP proteins, HEK293 T-REx WT cells were co-transfected with pDEST-MTS-HsCLPP-3×FLAG or pDEST-MTS-HsCLPP(S153A)-3×FLAG and pOG44 (Thermo-Fisher Scientific), using the jetPRIME In Vitro DNA Transfection Reagent (Polyplus Transfection). Stable cell populations were isolated by multiple rounds of selection in media supplemented with 200 μg/mL hygromycin.

HEK293 T-REx ΔCLPX* and HEK293 T-REx CLPP^{-/-} ΔCLPX* were generated by disrupting the CLPX gene in HEK293 T-REx WT and CLPP^{-/-} cells, respectively, using CRISPR-Cas9 methodology as detailed in Cong et al., 2013. Briefly, the required gRNA sequence targeting in proximity to exon 1 of CLPX was introduced into pX330 with the oligonucleotides CLPX Exon1 pX330 F and CLPX Exon1 pX330 R (sequence shown in Table 1) via designated BbsI restriction sites. Next, the modified pX330 plasmid and the oligonucleotide CLPX Exon1 ssODN (sequence shown in Table 1) were co-transfected into WT and CLPP^{-/-} cells using Lipofectamine 2000 (Thermo-Fisher Scientific) following the manufacturer's protocol. CLPX Exon1 ssODN provides the necessary repair template after the Cas9-mediated DNA cleavage and causes omission of CLPX's start codon to inhibit protein expression. The efficacy of CLPX disruption in suppressing HsCIpX expression was assessed by Western blotting. Both WT and CLPP were subjected to two additional rounds of CRISPR-Cas9 treatment in sequence to maximize the suppression of HsCIpX expression.

To assess the cytotoxicity of ADEP analogs, cells were seeded at 1×10³ or 2×10³ cells in 50 μL per well on sterile 96-well flat-bottom tissue culture plates (VWR). Growth media without any cells was included as a control. Cells were grown for at least 24 hours to allow proper adherence to the growth surface. Afterwards, ADEP analogs were serially diluted and introduced to the tissue cultures via fresh growth media at 50 μL per well. Inclusion of activators/DMSO brought the final DMSO concentration of the growth media up to a maximum of 0.5% (v:v). For experiments that involve overexpression of FLAG-tagged HsCIpP (WT or S153A mutant), Dox (Sigma-Aldrich) was also applied at 0.4 μg/mL (final concentration) as needed. A total of four independent replicates were prepared for each cell line and for each growth condition used. Cells were grown in the presence of ADEP/DMSO for 72 hours.

To assess cell survival, live adherent cells were fixed with 10% (w:v) trichloroacetic acid (TCA) at 4° C. for 1-2 hours. The tissue culture plates were then rinsed with water to remove dead cells and cell debris and air-dried overnight. Fixed cells were stained with a solution of 0.4% (w:v) sulforhodamine B (SRB) (Sigma-Aldrich) dissolved in 1% (v:v) acetic acid at room temperature for 30 minutes, followed by plate-rinsing with 1% acetic acid and air-drying. The SRB retained was extracted with 10 mM of Tris base (200 μL per sample). Absorbance at λ=510 nm (A₅₁₀) was then measured using the EnSpire 2300 Multilabel Reader. A₅₁₀ is linearly proportional to the amount of cellular protein present in each sample.

To determine the IC₅₀ for the ADEP analogs, relative cell viability (RCV) values were calculated by normalizing the A₅₁₀ readings in ADEP-containing sample to the DMSO-only control. Plots of RCV vs. ADEP concentration were constructed, followed by non-linear regression analysis using a standard dose response equation, to determine the required IC₅₀ values.

$$RCV = {RCV}_{min} + \frac{{RCV}_{max} - {RCV}_{min}}{1 + 1 {0}^{( log {IC}_{50} - log [ ADEP ] ) h}}$$

In this equation, RCVₘᵢₙ and RCVₘₐₓ are the minimum and maximum RCV observed, respectively; [ADEP] is the molar concentration of

ADEP applied; and h is the Hill coefficient. For HEK293 T-REx CLPP^{-/-} and HEK293 T-REx CLPP^{-/-} [symbol] CLPX* cells, IC₅₀ values were estimated by manual examination of the plotted data due to the lack of a minimum plateaus, which prohibited meaningful analysis using the Hill model.

### Mitochondrial Proteome Profiling for WT and CLPP^{-/-}

HEK293 T-REx WT and CLPP^{-/-} cells were grown to ˜80% confluence, with two biological replicates prepared for WT and three for CLPP^{-/-}. All cells were chemically crosslinked by treatment with 0.5 mM of the cell membrane-permeable crosslinker dithiobis succinimidyl propionate (DSP) for 30 min at room temperature. The crosslinking reagent was quenched from the samples by the addition and incubation with 100 mM Tris-HCl (pH 7.5) and 2 mM EDTA for 10 min at room temperature. Cells were suspended by gentle pipetting and harvested by centrifugation at 600×g for 5 min and then washed twice in ice-cold NKM buffer (10 mM TrisHCl pH 7.5, 130 mM NaCl, 5 mM KCl, and 7.5 mM MgCl₂). The pellet was resuspended in ice-cold buffer containing 10 mM TrisHCl (pH 6.8), 130 mM NaCl, 10 mM KCl, 150 mM MgCl₂, 1 mM PMSF, and 1 mM DTT. The resuspended cells were then allowed to swell for 10 min, followed by lysis via repeated passages through a syringe fitted with a 23-gauge needle. Cell lysates were added to 1 cell-pellet-volume of 2 M sucrose and centrifuged at 1,300×g for 5 min at 4° C. The resulting supernatant was further centrifuged at 7,000×g for 10 min at 4° C. to pellet the mitochondrial fraction.

To lyse the mitochondrial fraction and further alkylate the mitochondrial proteins, the pellet was dissolved in 8 M urea, 20 mM TrisHCl pH 7.5 and 8 mM chloroacetamide, and subjected to sonication for 30 s with 15-30 s cooling cycles for 3 min on ice. After incubating the lysate at room temperature for 30 min, 50 mM TrisHCl was added to dilute the urea concentration from 8 M to 2 M. To this mixture, 20 mM DTT and 5 μg/mg-of-sample of Trypsin Gold (Promega) were also added to trypsinize the proteins overnight at room temperature. Trypsin activity was stopped by adding 1 μL of trifluoroacetic acid until a pH of 2-3 was reached. After desalting the samples using the C18 packed tips (Glygen Corp), the bound peptides were eluted for the tips with 0.1% formic acid and 60% acetonitrile. Protein content for WT and CLPP^{-/-} samples was assessed by Bradford assay and adjusted to 0.9 mg. These were then dried and resuspended in 1% formic acid for mass spectrometry analysis.

All samples were analyzed by a Proxeon EASY nanoLC 1000 System (Thermo-Fisher Scientific) coupled to an Orbitrap Elite mass spectrometer (Thermo-Fisher Scientific). For chromatographic separation, 5 μL of WT and CLPP^{-/-} samples with same protein concentration was loaded onto an Acclaim PepMap C18 column (15 cm×50 μm ID, 3 μm, 100 A; Thermo-Fisher Scientific). Peptides were separated using the following elution gradients at time points in sequence: 0-3% B (0-2 min); 3-24% B (2-170 min); 24-100% B (172-190 min); 100% B (190-200 min). For this step, Buffer A refers to 0.1% formic acid and Buffer B refers to 0.1% formic acid in acetonitrile. Separation of peptides was achieved at a column flow rate of 0.30 μL/min. Eluted peptides were immediately ionized by positive electrospray ionization at an ion source temperature of 250° C. and an ion spray voltage of 2.1 kV. Full-scan MS spectra (m/z 350-2000) was acquired in the Orbitrap at mass resolution of 60000 (m/z 400) using the positive ion mode. The automatic gain control was set at 1 e6 for full FTMS scans and 5e4 for MS/MS scans. Fragmentation was performed with collision-induced dissociation (CID) in the linear ion trap with an ion intensity of >1500 counts. The 15 most intense ions isolated for ion trap CID with charge states ≥2 were sequentially fragmented using the normalized collision energy setting at 35%, activation Q at 0.250, and an activation time of 10 ms. Ions selected for MS/MS were dynamically excluded for 30 s.

The mass spectra files were then searched using MaxQuant ver. 1.6.1.0 (Max Planck Institute) against a UniProt human protein database of canonical sequences. The resulting data were filtered for contaminants and reverse matches. The protein intensities of the detected mitochondrial proteins were normalized against the pooled WT samples, and Student t-test was used to identify proteins with a 1.5-fold change (CLPP^{-/-}/WT) at a p-value significance 0.05.

### Light and Fluorescence Microscopy

HEK293 T-REx WT and CLPP^{-/-} cells were grown on glass cover slips placed in a 6-well tissue culture plate. Prior to use, the cover slips were cleaned by treatment with 1 M HCl for 2-3 days, followed by thorough rinsing with distilled water and a second treatment with 95% ethanol for 16-18 hours. The cleaned cover slips were sterilized by autoclaving and dried under UV light. To ensure proper attachment of the cells, the sterilized cover slips were coated with gelatin by application of a 0.2% solution and incubation at 37° C. for at least 2 hours. Excess gelatin solution was then removed, and the cover slips were allowed to dry in a sterile environment for 2-4 hours before use.

WT cells treated with ADEP-41 were seeded at a density of 1×10⁵ cells/mL (2 mL per well) to compensate for lack of cell growth and division resulting from the ADEP treatment. WT cells treated with DMSO and CLPP cells treated with ADEP-41 or DMSO were all seeded at 5×10⁴ cells/mL (2 mL per well). After the cells have adhered properly, ADEP-41 (20 μM for a 24-hour treatment; 10 μM for a 72-hour treatment) or DMSO was applied via media exchange (final DMSO concentration at 0.2% for all samples). Cells were then grown for 24 or 72 hours prior to fixing and permeabilization for microscopy.

Cells were fixed in the presence of 4% formaldehyde at room temperature for 30-45 minutes, followed by membrane permeabilization with 0.1% Triton X-100 (in PBS from Gibco) supplemented with 0.1% sodium citrate at room temperature for 30 minutes. All samples were rinsed gently with PBS in between steps. For the TUNEL (Terminal deoxynucleotidyl transferase dUTP nick end labelling) assay, the fixed and permeabilized cells were labelled using the In Situ Cell Death Detection Kit, Fluorescein (Roche-11684795910), following the manufacturer's instructions. Cells were mounted onto standard glass microscope slides with DAPI Fluoromount-G (Southern Biotech) for visual examination using the Eclipse 80i fluorescence microscope (Nikon) equipped with the X-Cite Series 120Q excitation light source (Excelitas Technologies). Both light and fluorescence microscopic images were captured and analyzed using the NIS-Elements Basic Research Software (Nikon).

### 3D Structured Illumination Microscopy (3DSIM)

HEK293 T-REx WT and CLPP^{'1/-} cells were seeded on coverslips (Electron Microscopy Sciences) pre-coated with poly-D-lysine hydrobromide (Sigma-Aldrich) in a 12-well culture plate (Falcon). Cells were grown for 24 hours to allow adhesion, followed by treatment with 2 μM ADEP-41 or DMSO in the same manner as described in previous sections, for 24 or 72 hours. At these time points, the original media was removed, and cells were incubated for 30 minutes at 37° C., 5% CO₂ with 200 nM MitoTracker Red CMXRos (Molecular Probes) in Opti-MEM™ Reduced Serum Medium (Gibco). Cells were then washed once with PBS and fixed with 4% (v/v) paraformaldehyde (Electron Microscopy Sciences) in PBS for 20 minutes at room temperature.

The fixed cells were then washed twice with PBS, permeabilized with 0.2% (v/v) Triton X-100 (BioShop) in PBS for 10 minutes at room temperature and incubated with 2 μg/mL Hoechst 33342 (Molecular Probes) in PBS for 10 minutes at room temperature to counterstain nuclei. Coverslips were then washed three times with PBS+0.05% (v/v) Tween-20 and mounted on microscope glass slides (VWR) using 0.5% (w/v) propyl gallate (Sigma-Aldrich) dissolved in glycerol as the mounting medium.

3DSIM data were collected on the ELYRA PS.1 super resolution microscope (Carl Zeiss Microscopy) using a 63× (1.4 NA) Plan-Apochromatic oil immersion objective (Carl Zeiss Microscopy) and a 1.6× Optovar. For imaging, fluorophores were laser-excited at wavelengths 405 nm and 555 nm, and the emissions were collected with band-pass 420-480, 570-620 filters, respectively. Z-stacks were acquired over a 10-μm thickness with 101 nanometre-steps, using an iXon 885 EMCCD camera (Andor). For each image field, grid excitation patterns were acquired for five phases and three rotation angles (−75°, −15°, +45°). Raw data were reconstructed using the SIM module of ZEN Black software version 8.1 (Carl Zeiss Microscopy), with a Wiener noise filter value of −5. The final images were obtained from the reconstructed data by using a maximum intensity projection (MIP) algorithm, implemented by the ZEN Black software.

### Western Blots

HEK293 T-REx WT, HEK293 T-REx CLPP^{-/-}, HeLa (regular), HeLa T-REx, U2OS and undifferentiated SH-SYSY cells were grown in the presence of ADEP or DMSO as described in previous sections. Cells were harvested by the standard trypsinization protocol and counted with a hemocytometer. Cell lysates were prepared by first re-suspending the cells in PBS at 5×10⁶ cells/mL, followed by sonication. Proteins in the lysates were analyzed by Western blotting using Immobilin-P PVDF membranes (EMD Millipore). Blots were imaged and analyzed with the ChemiDoc XRS+ System (BioRad). Quantification of HsCIpP expression was performed by densitometry using the Quantity One 1D Analysis Software (BioRad). HsCIpP expression levels were corrected for sample loading errors and normalized to the expression level in HEK293 T-REx WT. Primary antibodies for HsCIpP, HsCIpX and GAPDH were purchased from Abcam. Primary antibodies for Caspase-8, Caspase-9 and PUMA were purchased from Cell Signaling Technologies. The primary antibody for Caspase-3 was purchased from R&D Systems. Primary antibodies for Mcl-1 and Bcl-2 were purchased from EMD Millipore. The primary antibody for the FLAG-tag was purchased from Sigma-Aldrich. HRP-conjugated goat anti-rabbit IgG (for HsCIpX, Caspase-8, Caspase-9 and PUMA) and HRP-conjugated goat anti-mouse IgG (for HsCIpP, Mcl-1, Bcl-2, GAPDH and FLAG-tag) were purchased from BioRad. HRP-conjugated rabbit anti-goat IgG (for Caspase-3) was purchased from Sigma-Aldrich.

### Cellular Respiration Analysis

HEK293 T-REx WT and CLPP^{31 /-} cells were seeded onto Seahorse XF96 Cell Culture Microplates (Agilent) pre-coated with poly-D-lysine (Sigma-Aldrich). Adherent cells were then grown for 24 hours in the presence of 1 μM of ADEP-41 or DMSO as described in previous sections. Cellular respiration was assessed using the Seahorse XF Cell Mitochondrial Stress Kit (Agilent) following the manufacturer's protocol. Oxygen consumption rate (OCR) and extracellular acidification rate (ECAR) were monitored using the Seahorse XFe96 Analyzer (Agilent) fitted the XFe96 sensor cartridge (Agilent). Carbonyl cyanide-4-(trifluoromethoxy)phenylhydrazone (FCCP) that was included in the Mitochondrial Stress Kit was applied to the cell cultures at a final concentration of 0.25 μM. Application of other reagents were done as outlined in the manufacturer's experimental guidelines. Data analysis was performed with the Wave Desktop program (Agilent) following the manufacturer's instructions. For OCR and ECAR normalization, cells were first fixed with 4% formaldehyde as described in previous sections. Nuclear DNA of the fixed cells was stained with Hoechst 33342 (Invitrogen) dissolved in PBS at 5 μg/mL for 30 minutes at room temperature, followed by three rounds of washes with PBS. To measure Hoechst 33342 fluorescence, 50 μL of PBS was applied to each well, followed by fluorescence measurements (λₑₓ=350 nm; λₑₘ=461 nm) using the EnSpire 2300 Multilabel Reader. Fluorescence data were expressed arbitrarily in 10⁴ HFU (Hoechst fluorescence units) to keep the normalized OCR and ECAR within conventional numerical ranges. Operation of the Seahorse XFe96 Analyzer and data collection was performed by the SPARC BioCentre (Hospital for Sick Children, Toronto, Canada).

### Quantification and Statistical Analysis

Statistical analyses and software that were used for specific experiments have been described in the relevant subsections under materials and methods. In general, all quantitative data were collected from at least three independent replicates and are presented as means±SD. All enzyme kinetics experiments and cytotoxicity assays on mammalian cells were repeated multiple times to ensure data reproducibility. Quantitative microscopy data for TUNEL were derived from examining at least 50 cells per sample replicate per repeated experiment.

### Data and Software Availability

The structure of the ADEP-28-HsCIpP complex has been deposited in the Protein Data Bank (PDB) under the accession number 6BBA. All software used for the experiments presented here are available at the sources listed in the Table 1.

### Results

Identification of ADEP Analogs that Dysregulate HsCIpP

To identify ADEP analogs that allow HsCIpP to degrade folded proteins independent of HsCIpX+ATP (i.e., that activate or dysregulate HsCIpP), the inventors employed the in vitro screening method that was used for identifying small molecule activators of Escherichia coli CIpP (EcCIpP) (Leung et al., 2011). In this approach, the ability of HsCIpP to degrade FITC-labeled casein (casein-FITC) was measured and compared to the ability of EcCIpAP to degrade casein-FITC. This is quantitatively expressed as relative degradation (RD) index (Leung et al., 2011).

Forty-six ADEP analogs (Goodreid et al., 2016) were tested against HsCIpP (see FIGS. 1D-1I for structures). Initially, the analogs were screened at a final concentration of 25 μM and their RD25 scores (i.e., RD at 25 μM of compound) were measured (FIG. 1A). Top hits were then screened at 5 μM and then at 1 μM compound concentration (FIG. 1B and Table 3). For follow-up experiments, five ADEP analogs (FIG. 10) were chosen based on their diverse RD1 scores to provide a good representation across the potency spectrum. Furthermore, the selected ADEP analogs have distinct structural differences (FIG. 1C).

TABLE 3
RD25. RD5 and RD1 scores of ADEP analogs in HsCIpP

| | Hs | Hs | |
|---|---|---|---|
| Compound | RD25 | SD | SMILES |
| ADEP-02 | 1.08 | 0.08 | O═C([C@H]C)NC([C@@]1([H])N(CCCC1)C([C@@]2([H]) |
| | | | N(C([C@@H](NC([C@H](CC3═CC═CC═C3) |
| | | | NC(/C═C/CC═OCC)═O)═O)CO4)═O)CCC2)═O)═O) |
| | | | N5CCC[C@@]5([H])C4═O |
| ADEP-10 | 1.07 | 0.03 | O═([C@H](C)NC([C@@]1([H])N(CCCC1)C([C@@]2([H]) |
| | | | N(C([C@@H](NC([C@H](CC3═CC═CC═C3) |
| | | | NC(/C═C/CCCC)═O)═O)CO4)═O)CCC2)═O)═O) |
| | | | N5C[C@H](C)C[C@@]5([H])C4═O |
| ADEP-18 | 1.07 | 0.04 | O═C([C@H](C)NC([C@H](C)N(C)C([C@@] |
| | | | 1([H])N(C([C@@H](NC([C@H](CC2═CC═CC═C2) |
| | | | NC(/C═C/CCCCC)═O)═O)CO3)═O)CCC1)═O)═O) |
| | | | N4CCC[C@@]4([H])C3═O |
| ADEP-06 | 1.05 | 0.04 | O═C([C@H](C)NC([C@H](C)N(C)C([C@@]1([H]) |
| | | | N(C([C@@H](NC([C@H](CC2═CC(F)═CC(F)═C2) |
| | | | NC(/C═C/CCCC)═O)═O)CO3)═O)CCC1)═O)═O) |
| | | | N4C[C@H](C)C[C@@]4([H])C3═O |
| ADEP-15 | 1.05 | 0.02 | O═C([C@H](C)NC([C@@](CCC1)([H])N1C([C@@]2([H]) |
| | | | N(C([C@@H](NC([C@H](CC3═CC(F)═CC(F)═C3)NC |
| | | | (/C═C/CCCC)═O)═O)CO4)═O)CCC2)═O)═O)N5C[C@H] |
| | | | (C)C[C@@]5([H])C4═O |
| ADEP-20 | 1.04 | 0.03 | O═C([C@H](C)NC([C@@]1([H])N(CCCC1)C([C@@]2([H]) |
| | | | N(C([C@@H](NC([C@H](CC3═CC(F)═CC═C3) |
| | | | NC(/C═C/CCC)═O)═O)CO4)═O)CCC2)═O)═O)N5C |
| | | | [C@N(C)C[C@@]5([H])C4═O |
| ADEP-32 | 1.03 | 0.11 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CCCC)═O)═O)CN4)═O)CCC2)═O)═O)N5C[C@N(C) |
| | | | C[C@@]5([H])C4═O |
| ADEP-04 | 1.03 | 0.02 | O═C([C@H](C)NC([C@H](C)N(C)C([C@@]1([H])N(C([C@@H] |
| | | | (NC([C@H](CC2═CC═CC═C2)NC(/C═C/C═C/CCC)═O)═O) |
| | | | CO3)═O)CCC1)═O)═O)N4C[C@N(C)C[C@@]4([H])C3═O |
| ADEP-13 | 1.02 | 0.02 | O═C([C@H](C)NC([C@@]1([H])N(CCCC1)C([C@@]2([H]) |
| | | | N(C([C@@H](NC([C@H](CC3═CC(F)═CC(F)═C3) |
| | | | NC(/C═C/CC4CC4)═O)═O)[C@N(C)O5)═O)CCC2)═O)═O) |
| | | | N6C[C@N(C)C[C@@]6([H])C5═O |
| ADEP-17 | 1.02 | 0.02 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CC4CCCCC4)═O)═O)[C@H](C)O5)═O)CCC2)═O)═O) |
| | | | N6C[C@N(C)C[C@@]6([H])C5═O |
| ADEP-21 | 1.00 | 0.03 | O═C([C@H](C)NC([C@@]1([H])N(CCCC1)C([C@@]2([H]) |
| | | | N(C([C@@H](NC([C@H](CC3CCCCC3)NC |
| | | | (/C═C/CCCC)═O)═O)CO4)═O)CCC2)═O)═O)N5C |
| | | | [C@H](C)C[C@@]5([H])C4═O |
| ADEP-14 | 1.00 | 0.03 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/C═C/CCC)═O)═O)[C@H](C)O4)═O)CCC2)═O)═O)N5C |
| | | | [C@H](C)C[C@@]5([H])C4═O |
| ADEP-30 | 0.98 | 0.05 | O═C([C@H](C)NC([C@@]1([H])N(CC[C@H](C)C1)C([C@@]2 |
| | | | ([H])N(C([C@@H](NC([C@H](CC3═CC(F)═CC(F)═C3) |
| | | | NC(/C═C/CCCC)═O)═O)CO4)═O)CCC2)═O)═O)N5C[C@H](C) |
| | | | C[C@@]5([H])C4═O |
| ADEP-05 | 0.98 | 0.02 | O═([C@H](C)NC([C@H](C)N(C)C([C@@]1([H])N |
| | | | (C([C@@H]( N C([C@H](CC2═CC═CC═C2)NC |
| | | | (/C═C/C ═C/C ═C/C)═O)═O)CO3)═O)CCC1)═O)═O) |
| | | | N4C[C@N(C)C[C@@]4([H])C3═O |
| ADEP-25 | 0.97 | 0.04 | O═C([C@H(CC)NC([C@@]1([H])N(CCCC1)C([C@@]2([H]) |
| | | | N(C([C([C@@H](NC([C@H](CC3═CC(F)═CC(F)═C3)NC |
| | | | (/C═C/CCCC)═O)═O)CO4)═O)CCC2)═O)═O)N5C[C@H](C)C |
| | | | [C@@]5([H])C4═O |
| ADEP-08 | 0.97 | 0.03 | O═([C@H](C)NC([C@H](C)N(C)C([C@@]1([H])N |
| | | | (C([C@@H](NC([C@H](CC2═CC═CC═C2)NC |
| | | | (/C═C/C═C/C═C/C)═O)═O)CO3)═O)CCC1)═O)═O) |
| | | | N4CCC[C@@]4([H])C3═O |
| ADEP-41 | 0.96 | 0.04 | O═([C@H](C)NC([C@@]1([H])N(CCCC1)C([C@@]2 |
| | | | ([H])N(C([C@@H](NC([C@H](CC3═CC(F)═CC(F)═C3)NC |
| | | | (/C═C/CCCC)═O)═O)[C@N(C)O4)═O)CCC2)═O)═O)N5CCC |
| | | | [C@@]5([H])C4═O |
| ADEP-01 | 0.96 | 0.04 | O═([C@H](C)NC([C@H](C)N(C)C([C@@]1([H])N |
| | | | (C([C@@H](NC([C@H](CC2═CC═CC═C2)NC |
| | | | (/C═C/CCCC)═O)═O)CO3)═O)CCC1)═O)═O)N4CCC |
| | | | [C@@]4([H])C3═O |
| ADEP-28 | 0.95 | 0.04 | O═([C@H](C)NC([C@@]1([H])N(CC[C@H](C)C1)C |
| | | | ([C@@]2([H])N(C([C@@H](NC([C@H] |
| | | | (CC3═CC(F)═CC(F)═C3)NC(/C═C/CCCC)═O)═O) |
| | | | [C@H](C)O4)═O)CCC2)═O)═O)N5CCC[C@@]5([H])C4═O |
| ADEP-38 | 0.95 | 0.04 | O═([C@H](C)NC([C@@]1([H])N(CC[C@H](C)C1)C([C@@]2 |
| | | | ([H])N(C([C@@H](NC([C@H](CC3═CC(F)═CC(F)═C3 |
| | | | NC(/C═C/CCCC)═O)═O)[C@H](C)O4)═O)CCC2)═O)═O)N |
| | | | 5C[C@N(C)C[C@@]5([H])C4═O |
| ADEP-37 | 0.92 | 0.04 | O═C([C@H](C)NC([C@@]1([H])N(CCC2═C1C═CC═C2) |
| | | | C([C@@]3([H])N(C([C@@H](NC([C@H](CC4═CC(F)═CC |
| | | | (F)═C4)NC(/C═C/CCCC)═O)═O)CO5)═O)CCC3)═O)═O)N6C |
| | | | [C@H](C)C[C@@]6([H])C5═O |
| ADEP-29 | 0.92 | 0.04 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CCCC)═O)═O)[C@N(C)O4)═O)CCC2)═O)═O) |
| | | | N5C[C@H](C)C[C@@]5([H])C4═O |
| ADEP-43 | 0.87 | 0.03 | O═C([C@H](C)NC([C@@]1([H])N(CCCC1)C([C@@]2([H]) |
| | | | N(C([C@@H](NC([C@H](CC3═CC(C═CC═C4)═C4C═C3) |
| | | | NC(/C═C/CCCC)═O)═O)CO5)═O)CCC2)═O)═O)N6C[C@H] |
| | | | (C)C[C@@]6([H])C5═O |
| ADEP-42 | 0.87 | 0.02 | O═C([C@H](C)NC([C@@]1([H])N(CCCC1)C([C@@]2([H]) |
| | | | N(C([C@@H](NC([C@H](CC3═CSC4═C3C═CC═C4)NC |
| | | | (/C═C/CCCC)═O)═O)CO5)═O)CCC2)═O)═O)N6C[C@N(C) |
| | | | C[C@@]6([H])C5═O |
| ADEP-26 | 0.86 | 0.02 | O═C([C@H](C)NC([C@H](C)N(C)C([C@@]1([H]) |
| | | | N(C([C@@H](NC([C@H](CC2═CC═CC═C2)NC |
| | | | (/C═C/C═C/C)═O)═O)CO3)═O)CCC1)═O)═O) |
| | | | N4CCC[C@@]4([H])C3═O |
| ADEP-39 | 0.78 | 0.04 | O═C([C@H](C)NC([C@@]1([H])N(CC[C@H](C)C1) |
| | | | C([C@@]2([H])N(C([C@@H](NC([C@H] |
| | | | (CC3═CC(F)═CC(F)═C3)NC(/C═C/C4═CC═CC |
| | | | (C(F)(F)F)═C4)═O)═O)[C@H](C)O5)═O)CCC2)═O)═O) |
| | | | N6C[C@N(C)C[C@@]6([H])C5═O |
| ADEP-11 | 0.77 | 0.03 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CCCC)═O)═O)CO4)═O)CCC2)═O)═O)N5C |
| | | | [C@N(C)C[C@@]5([H])C4═O |
| ADEP-09 | 0.76 | 0.03 | O═C([C@H](C)NC([C@H](C)N(C)C([C@@]1([H])N |
| | | | (C([C@@H](NC([C@H](CC2═CC═CC═C2)NC |
| | | | (/C═C/CCC)═O)═O)CO3)═O)CCC1)═O)═O) |
| | | | N4CCC[C@@]4([H])C3═O |
| ADEP-03 | 0.66 | 0.03 | O═C([C@H](C)NC([C@H](C)N(C)C([C@@]1([H]) |
| | | | N(C([C@@H](NC([C@H](CC2═CC═CC═C2)NC |
| | | | (/C═C/C═C/C)═O)═O)CO3)═O)CCC1)═O)═O) |
| | | | N4C[C@N(C)C[C@@]4([H])C3═O |
| ADEP-27 | 0.64 | 0.02 | O═C([C@H](C)NC([C@H](C)N(C)C([C@@]1 |
| | | | ([H])N(C([C@@H](NC([C@H](CC2═CC═CC═C2) |
| | | | NC(C#CCCCCC)═O)═O)CO3)═O)CCC1)═O)═O) |
| | | | N4CCC[C@@]4([H])C3═O |
| ADEP-07 | 0.61 | 0.02 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CCCCC(O)═O)═O)═O)[C@N(C)O4)═O)CCC2)═O)═O) |
| | | | N5C[C@N(C)C[C@@]5([H])C4═O |
| ADEP-44 | 0.42 | 0.02 | O═([C@H](C)NC([C@@]1([H])N(CC[C@H](C)C1)C([C@ |
| | | | @]2([H])N(C([C@@H](NC([C@H](CC3═CC(F)═CC(F)═C3) |
| | | | NC(/C═C/C4═CC═CC═C4C(F)(F)F)═O)═O) |
| | | | [C@N(C)O5)═O)CCC2)═O)═O) |
| | | | N6C[C@H](C)C[C@@]6([H])C5═O |
| ADEP-12 | 0.36 | 0.02 | O═([C@H](C)NC([C@H](C)N(C)C([C@@]1([H])N |
| | | | (C([C@@H](NC([C@H](CC2═CC═CC═C2)NC |
| | | | (C#CCCCC)═O)═O)CO3)═O)CCC1)═O)═O) |
| | | | N4CCC[C@@]4([H])C3═O |
| ADEP-19 | 0.34 | 0.02 | O═([C@H](C)NC([C@H](C)N(C)C([C@@]1([H])N |
| | | | (C([C@@H](NC([C@H](CC2═CC═CC═C2)NC |
| | | | (/C═C\CCCC)═O)═O)CO3)═O)CCC1)═O)═O) |
| | | | N4CCC[C@@]4([H])C3═OO═([C@H](C)NC |
| | | | ([C@@]1([H])N(CCCC1)C([C@@]2([H]) |
| ADEP-45 | 0.29 | 0.01 | N(C([C@@H](NC([C@H](CC(C(F)═C3F)═C(F)C(F)═C3F) |
| | | | NC(/C═C/CCCC)═O)═O)CO4)═O)CCC2)═O)═O) |
| | | | N5C[C@H](C)C[C@@]5([H])C4═O |
| ADEP-23 | 0.12 | 0.02 | O═([C@H](C)NC([C@H](C)N(C)C([C@@]1([H])N |
| | | | (C([C@@H](NC([C@H](CC2═CC═CC═C2)NC |
| | | | (C#CCCC)═O)═O)CO3)═O)CCC1)═O)═O) |
| | | | N4CCC[C@@]4([H])C3═O |
| ADEP-33 | 0.07 | 0.01 | O═([C@H](C)NC([C@H](C)N(C)C([C@@]1([H])N |
| | | | (C([C@@H](NC([C@H](CC2═CC═CC═C2)NC |
| | | | (/C═C\CCC)═O)═O)CO3)═O)CCC1)═O)═O) |
| | | | N4CCC[C@@]4([H])C3═O |
| ADEP-16 | 0.06 | 0.13 | O═([C@H](C)NC([C@H](C)N(C)C([C@@]1([H]) |
| | | | N(C([C@@H]( N C([C@H](CC2═CC═CC═C2)NC |
| | | | (OCC3═CC═CC═C3)═O)═O)CO4)═O)CCC1)═O)═O) |
| | | | N5C[C@N(C)C[C@@]5([H])C4═O |
| ADEP-46 | 0.02 | 0.02 | O═([C@H](C)NC([C@H](C)N(C)C([C@@]1([H])N |
| | | | (C([C@@H]( N C([C@H](CC2═CC ═CC ═C2)NC |
| | | | (/C═C/C ═C/CC3(H])OCCCC3)═O)═O)CO4)═O) |
| | | | CCC1)═O)═O)N5C[C@H](C)C[C@@]5([H])C4═O |
| ADEP-36 | 0.00 | | O═([C@H](C)NC([C@H](C)N(C)C([C@@]1([H])N |
| | | | (C([C@@H](NC([C@H](CC2═CC═CC═C2) |
| | | | NC(/C═C/C═C/CO)═O)═O)CO3)═O)CCC1)═O)═O) |
| | | | N4C[C@H](C)C[C@@]4([H])C3═O |
| ADEP-24 | 0.00 | | O═([C@H](C)NC([C@@]1([H])N(CCCC1)C([C@@]2([H]) |
| | | | N(C([C@@H](NC([C@H](CC3═CC═CC═C3)NC |
| | | | (C4═CC═C([N+]([O−])═O)C═C4)═O)═O) |
| | | | CO5)═O)CCC2)═O)═O)N6CCC[C@@]6([H])C5═O |
| ADEP-34 | 0.00 | | O═([C@H](C)NC([C@H](C)N(C)C([C@@]1([H])N(C |
| | | | ([C@@H](NC([C@H](CC2═CC═CC═C2)NC |
| | | | (OC(C)(C)C)═O)═O)CO3)═O)CCC1)═O)═O) |
| | | | N4CCC[C@@]4([H])C3═O |
| ADEP-35 | 0.00 | | O═([C@H](C)NC([C@H](C)N(C)C([C@@]1([H])N(C([C@@H] |
| | | | (NC([C@H](CC2═CC═CC═C2)NC(OC(C)(C)C)═O)═O) |
| | | | CO3)═O)CCC1)═O)═O)N4C[C@H](C)C[C@@]4([H])C3═O |
| ADEP-31 | 0.00 | | O═([C@H](C)NC([C@H](C)N(C)C([C@@]1([H])N(C([C@@H] |
| | | | (NC([C@H](CC2═CC═CC═C2)N)═O)CO3)═O)CCC1)═O)═O) |
| | | | N4C[C@N(C)C[C@@]4([H])C3═O |
| ADEP-22 | 0.00 | | O═([C@H](C)NC([C@H](C)N(C)C([C@@]1([H])N |
| | | | (C([C@@H](NC([C@H](CC2═CC═CC═C2)NC |
| | | | (OCC3═CC═CC═C3)═O)═O)CO4)═O)CCC1)═O)═O) |
| | | | N5CCC[C@@]5([H])C4═O |
| ADEP-40 | 0.00 | | O═([C@H](C)NC([C@H](C)N(C)C([C@@]1([H])N(C([C@@H] |
| | | | (NC([C@H](CC2═CC═CC═C2)N)═O)CO3)═O) |
| | | | CCC1)═O)═O)N4CCC[C@@]4([H])C3═O |
| | Hs | Hs | |
| Compound | RD5 | SD | SMILES |
| ADEP-32 | 0.96 | 0.03 | O═([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CCCC)═O)═O)CN4)═O)CCC2)═O)═O)N5C |
| | | | [C@N(C)C[C@@]5([H])C4═O |
| ADEP-06 | 0.94 | 0.02 | O═([C@H](C)NC([C@H](C)N(C)C([C@@]1([H])N |
| | | | (C([C@@H](NC([C@H](CC2═CC(F)═CC(F )═C2)NC |
| | | | (/C═C/CCCC)═O)═O)CO3)═O)CCC1)═O)═O)N4C |
| | | | [C@N(C)C[C@@]4([H])C3═O |
| ADEP-30 | 0.93 | 0.02 | O═([C@H](C)NC([C@@]1([H])N(CC[C@H](C)C1)C |
| | | | ([C@@]2([H])N(C([C@@H](NC([C@H](CC3═CC(F)═CC(F)═C3) |
| | | | NC(/C═C/CCCC)═O)═O)CO4)═O)CCC2)═O)═O) |
| | | | N5C[C@H](C)C[C@@]5([H])C4═O |
| ADEP-41 | 0.93 | 0.03 | O═([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CCCC)═O)═O)[C@N(C)O4)═O)CCC2)═O)═O) |
| | | | N5CCC[C@@]5([H])C4═O |
| ADEP-28 | 0.93 | 0.05 | O═C([C@H](C)NC([C@@]1([H])N(CC[C@H](C)C1)C |
| | | | ([C@@]2([H])N(C([C@@H](NC([C@H] |
| | | | (CC3═CC(F)═CC(F)═C3)NC(/C═C/CCCC)═O)═O) |
| | | | [C@H](C)O4)═O)CCC2)═O)═O)N5CCC[C@@]5([H])C4═O |
| ADEP-25 | 0.92 | 0.02 | O═C([C@H](C)NC([C@@]1([H])N(CCCC1)C([C@@]2([H]) |
| | | | N(C([CC@@H](NC([C@H](CC3═CC(F)═CC(F)═C3)NC |
| | | | (/C═C/CCCC)═O)═O)CO4)═O)CCC2)═O)═O) |
| | | | N5C[C@H](C)C[C@@]5([H])C4═O |
| ADEP-14 | 0.92 | 0.02 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/C═C/CCC)═O)═O)[C@H](C)O4)═O)CCC2)═O)═O)N5 |
| | | | C[C@H](C)C[C@@]5([H])C4═O |
| ADEP-15 | 0.92 | 0.02 | O═C([C@H](C)NC([C@@](CCC1)([H])N1C([C@@]2([H])N |
| | | | (C([C@@H](NC([CC@I-INC3═CC(F)═CC(F)═C3)NC(/C═C/ |
| | | | CCCC)═O)═O)CO4)═O)CCC2)═O)═O)N5C[C@H](C)C[C@ |
| | | | @]5([H])C4═O |
| ADEP-13 | 0.91 | 0.03 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CC4CC4)═O)═O)[C@N(C)O5)═O)CCC2)═O)═O) |
| | | | N6C[C@N(C)C[C@@]6([H])C5═O |
| ADEP-17 | 0.91 | 0.02 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CC4CCCCC4)═O)═O)[C@H](C)O5)═O)CCC2)═O)═O) |
| | | | N6C[C@N(C)C[C@@]6([H])C5═O |
| ADEP-20 | 0.90 | 0.03 | O═C([C@H](C)NC([C@@]1([H])N(CCCC1)C([C@@]2([H]) |
| | | | N(C([C@@H](NC([C@H](CC3═CC(F)═CC═C3)NC |
| | | | (/C═C/CCC)═O)═O)CO4)═O)CCC2)═O)═O)N5C |
| | | | [C@N(C)C[C@@]5([H])C4═O |
| ADEP-38 | 0.89 | 0.03 | O═C([C@H](C)NC([C@@]1([H])N(CC[C@H](C)C1)C([C@@]2 |
| | | | ([H])N(C([C@@H](NC([C@H](CC3═CC(F)═CC(F)═C3)NC |
| | | | (/C═C/CCCC)═O)═O)[C@H](C)O4)═O)CCC2)═O)═O)N |
| | | | 5C[C@N(C)C[C@@]5([H])C4═O |
| ADEP-02 | 0.88 | 0.02 | O═C([C@H](C)NC([C@@]1([H])N(CCCC1)C([C@@]2([H]) |
| | | | N(C([C@@H](NC([C@H](CC3═CC═CC═C3)NC |
| | | | (/C═C/CCCC)═O)═O)CO4)═O)CCC2)═O)═O) |
| | | | N5CCC[C@@]5([H])C4═O |
| ADEP-29 | 0.87 | 0.03 | O═C([C@H](C)NC([C@@]1([H])N(CCCC1)C([C@@]2([H]) |
| | | | N(C([C@@H](NC([C@H](CC3═CC(F)═CC(F)═C3)NC |
| | | | (/C═C/CCCC)═O)═O)[C@N(C)O4)═O)CCC2)═O)═O) |
| | | | N5C[C@H](C)C[C@@]5([H])C4═O |
| ADEP-37 | 0.86 | 0.03 | O═C([C@H](C)NC([C@@]1([H])N(CCC2═C1C═CC═C2) |
| | | | C([C@@]3([H])N(C([C@@H](NC([C@H] |
| | | | (CC4═CC(F)═CC(F)═C4)NC |
| | | | (/C═C/CCCC)═O)═O)CO5)═O)CCC3)═O)═O) |
| | | | N6C[C@H](C)C[C@@]6([H])C5═O |
| ADEP-10 | 0.85 | 0.03 | O═C([C@H](C)NC([C@@]1([H])N(CCCC1)C([C@@]2([H]) |
| | | | N(C([C@@H](NC([C@H](CC3═CC═CC═C3)NC |
| | | | (/C═C/CCCC)═O)═O)CO4)═O)CCC2)═O)═O) |
| | | | N5C[C@N(C)C[C@@]5([H])C4═O |
| ADEP-O4 | 0.78 | 0.02 | O═C([C@H](C)NC([C@H](C)N(C)C([C@@]1([H])N |
| | | | (C([C@@H]( N C([C@H](CC2═CC═CC═C2)NC |
| | | | (/C═C/C═C/CCC )═O)═O)CO3)═O)CCC1)═O)═O) |
| | | | N4C[C@N(C)C[C@@]4([H])C3═O |
| ADEP-18 | 0.72 | 0.02 | O═C([C@H](C)NC([C@H](C)N(C)C([C@@]1([H]) |
| | | | N(C([C@@H](NC([C@H](CC2═CC═CC═C2)NC |
| | | | (/C═C/CCCCC)═O)═O)CO3)═O)CCC1)═O)═O) |
| | | | N4CCC[C@@]4([H])C3═O |
| ADEP-21 | 0.70 | 0.02 | O═C([C@H](C)NC([C@@]1([H])N(CCCC1)C([C@@]2([H]) |
| | | | N(C([C@@H](NC([C@H](CC3CCCCC3) |
| | | | NC(/C═C/CCCC)═O)═O)CO4)═O)CCC2)═O)═O) |
| | | | N5C[C@H](C)C[C@@]5([H])C4═O |
| ADEP-01 | 0.70 | 0.02 | O═C([C@H](C)NC([C@H](C)N(C)C([C@@]1([H]) |
| | | | N(C([C@@H](NC([C@H](CC2═CC═CC═C2) |
| | | | NC(/C═C/CCCC)═O)═O)CO3)═O)CCC1)═O)═O) |
| | | | N4CCC[C@@]4([H])C3═O |
| ADEP-05 | 0.69 | 0.02 | O═C([C@H](C)NC([C@H](C)N(C)C([C@@]1([H])N |
| | | | (C([C@@H](NC([C@H](CC2═CC═CC═C2) |
| | | | NC(/C═C/C═C/C═C/C)═O)═O)CO3)═O)CCC1)═O)═O) |
| | | | N4C[C@N(C)C[C@@]4([H])C3═O |
| ADEP-43 | 0.55 | 0.02 | O═C([C@H](C)NC([C@@]1([H])N(CCCC1)C([C@@]2([H]) |
| | | | N(C([C@@H](NC([C@H](CC3═CC(C═CC═C4)═C4C═C3) |
| | | | NC(/C═C/CCCC)═O)═O)CO5)═O)CCC2)═O)═O)N6C[C@H] |
| | | | (C)C[C@@]6([H])C5═O |
| ADEP-08 | 0.52 | 0.02 | O═C([C@H](C)NC([C@H](C)N(C)C([C@@]1([H])N |
| | | | (C([C@@H](NC([C@H](CC2═CC═CC═C2) |
| | | | NC(/C═C/C═C/C═C/C)═O)═O)CO3)═O)CCC1)═O)═O) |
| | | | N4CCC[C@@]4([H])C3═O |
| ADEP-42 | 0.49 | 0.01 | O═C([C@H](C)NC([C@@]1([H])N(CCCC1)C([C@@]2([H]) |
| | | | N(C([C@@H](NC([C@H](CC3═CSC4═C3C═CC═C4)NC |
| | | | (/C═C/CCCC)═O)═O)CO5)═O)CCC2)═O)═O)N6C[C@N(C) |
| | | | C[C@@]6([H])C5═O |
| | Hs | Hs | |
| Compound | RD1 | SD | SMILES |
| ADEP-17 | 0.86 | 0.02 | O═C(N1C[C@@H](C[C@]12[H])CAC@@H](NC([C@] |
| | | | 3(N(C([C@]4(N(CCC4)C([C@H]([C@@H](OC2═O)C) |
| | | | NC([C@@H](NC(/C═C/CC5CCCCC5)═O) |
| | | | CC6═CC(F)═CC(F)═C6)═O)═O)[H])═O)CCCC3)[H])═O)C |
| ADEP-28 | 0.82 | 0.03 | O═C(N1CCC[C@]12[H])[C@@H](NC([C@]3(N(C([C@] |
| | | | 4(N(CCC4)C([C@H]([C@@H](OC2═O)C)NC([C@@H] |
| | | | (NC(/C═C/CCCC)═O)CC5═CC(F)═CC(F)═C5)═O)═O)[H])═O) |
| | | | CC[C@@H](C3)C)[H])═O)C |
| ADEP-14 | 0.79 | 0.02 | O═C(N1C[C@@N(C[C@]12[H])C)[C@@H](NC([C@] |
| | | | 3(N(C([C@]4(N(CCC4)C([C@H]([C@@H](OC2═O)C) |
| | | | NC([C@@H](NC(/C═C/C═C/CCC)═O) |
| | | | CC5═CC(F)═CC(F)═C5)═O)═O)[H])═O)CCCC3)[H])═O)C |
| ADEP-41 | 0.79 | 0.02 | O═C(N1CCC[C@]12[H])[C@@H](NC([CC@]3(N(C([C@] |
| | | | 4(N(CCC4)C([C@H]([C@@H](OC2═O)C)NC([CC@@H] |
| | | | (NC(/C═C/CCCC)═O)CC5═CC(F)═CC(F)═C5)═O)═O) |
| | | | [H])═O)CCCC3)[H])═O)C |
| ADEP-32 | 0.79 | 0.02 | O═C(N1C[C@@N(C[C@]12[H])C)[C@@H](NC([C@] |
| | | | 3(N(C([C@]4(N(CCC4)C([C@H](CNC2═O)NC([C@@H] |
| | | | (NC(/C═C/CCCC)═O)CC5═CC(F)═CC(F)═C5)═O)═O)[H])═O) |
| | | | CCCC3)[H])═O)C |
| ADEP-29 | 0.76 | 0.02 | O═C(N1C[C@@N(C[C@]12[H])C)[C@@H](NC([C@] |
| | | | 3(N(C([CC@]4(N(CCC4)C([C@H]([C@@H](OC2═O)C) |
| | | | NC([C@@H](NC(/C═C/CCCC)═O) |
| | | | CC5═CC(F)═CC(F)═C5)═O)═O)H])═O)CCCC3)[H])═O)C |
| ADEP-38 | 0.76 | 0.03 | O═C(N1C[C@@N(C[C@]12[H])C)[C@@H](NC([C@] |
| | | | 3(N(C([C@]4(N(CCC4)C([C@H]([C@@H](OC2═O)C) |
| | | | NC([C@@H](NC(/C═C/CCCC)═O) |
| | | | CC5═CC(F )═CC(F)═C5)═O)═O)H])═O) |
| | | | CC[C@@N(C3)CAN)═O)C |
| ADEP-30 | 0.69 | 0.02 | O═C(N1C[C@@N(C[C@]12[H])C)[C@@H](NC([C@] |
| | | | 3(N(C([C@]4(N(CCC4)C([C@H](COC2═O)NC([C@@H] |
| | | | (NC(/C═C/CCCC)═O)CC5═CC(F)═CC(F)═C5)═O)═O) |
| | | | [H])═O)CC[C@@H](C3)C)[H])═O)C |
| ADEP-37 | 0.68 | 0.03 | O═C(N1C[C@@N(C[C@]12[H])C)[C@@H](NC([C@] |
| | | | 3(N(C([C@]4(N(CCC4)C([C@H](COC2═O)NC([C@@H] |
| | | | (NC(/C═C/CCCC)═O)CC5═CC(F)═CC(F)═C5)═O)═O) |
| | | | [H])═O)CCC6═C3C═CC═C6)[H])═O)C |
| ADEP-06 | 0.64 | 0.02 | O═C(N1C[C@@N(C[C@]12[H])C)[C@@H](NC([C@@H] |
| | | | (N(C([CC@]3(N(CCC3)C([C@H](COC2═O)NC([CC@@H] |
| | | | (NC(/C═C/CCCC)═O)CC4═CC(F)═CC(F)═C4)═O)═O) |
| | | | [H])═O)C)C)═O)C |
| ADEP-25 | 0.61 | 0.02 | O═C(N1C[C@@N(C[C@]12[H])C)[C@@H](NC([C@] |
| | | | 3(N(C([C@]4(N(CCC4)C([C@H](COC2═O)NC([C@@H] |
| | | | (NC(/C═C/CCCC)═O)CC5═CC(F)═CC(F)═C5)═O)═O) |
| | | | [H])═O)CCCC3)[H])═O)CC |
| ADEP-13 | 0.59 | 0.02 | O═C(N1C[C@@N(C[C@]12[H])C)[C@@H](NC([C@] |
| | | | 3(N(C([C@]4(N(CCC4)C([C@H]([C@@H](OC2═O)C) |
| | | | NC([C@@H](NC(/C═C/CC5CC5)═O) |
| | | | CC6═CC(F)═CC(F)═C6)═O)═O)[H])═O)CCCC3)[H])═O)C |
| ADEP-15 | 0.58 | 0.02 | O═C(N1C[C@@H](C[C@]12[H])C)[C@@H](NC([C@] |
| | | | 3([H])CCCN3C([C@]4(N(CCC4)C([C@H](COC2═O) |
| | | | NC([C@H])NC(/C═C/CCCC)═O)CC5═CC |
| | | | (F)═CC(F)═C5)═O)═O)[H])═O)═O)C |
| ADEP-20 | 0.53 | 0.02 | O═C(N1C[C@@H](C[C@]12[H])CAC@@H](NC([C@] |
| | | | 3(N(C([C@]4(N(CCC4)C([C@H](COC2═O)NC([C@H] |
| | | | (NC(/C═C/CCC)═O)CC5═CC(F)═CC═C5)═O)═O)[H])═O) |
| | | | CCCC3)[HO═O)C |
| ADEP-02 | 0.52 | 0.02 | O═C(N1CCC[C@]12[H])[C@@H](NC([C@]3(N(C([C@] |
| | | | 4(N(CCC4)C([C@H](COC2═O)NC([C@H] |
| | | | (NC(/C═C/CCCC)═O)CC5═CC═CC═C5)═O)═O) |
| | | | [H])═O)CCCC3)[H])═O)C |
| ADEP-10 | 0.44 | 0.02 | O═C(N1C[C@@H](C[C@]12[H])CAC@@H](NC([C@] |
| | | | 3(N(C([C@]4(N(CCC4)C([C@H](COC2═O)NC([C@H] |
| | | | (NC(/C═C/CCCC)═O)CC5═CC═CC═C5)═O)═O)[H])═O) |
| | | | CCCC3)[H])═O)C |
| ADEP-O4 | 0.32 | 0.01 | O═C(N1C[C@@H](C[C@]12[H])CAC@@H](NC([C@@H] |
| | | | (N(C([C@]3(N(CCC3)C([C@H](COC2═O)NC([C@H] |
| | | | (NC(/C═C/C═C/CCC)═O)CC4═CC═CC═C4)═O)═O) |
| | | | [H])═O)C)C)═O)C |
| ADEP-01 | 0.22 | 0.01 | O═C(N1CCC[C@]12[H])[C@@H](NC([C@@H] |
| | | | (N(C([C@]3(N(CCC3)C([C@H](COC2═O) |
| | | | NC([C@H](NC(/C═C/CCCC)═O)CC4═CC═CC═C4)═O)═O) |
| | | | [H])═O)C)C)═O)C |

RD25 refers to the RD value of the ADEP analog at 25 μM as a measure of its ability to activate HsCIpP.
RDS refers to the RD value of the ADEP analog at 5 μM as a measure of its ability to activate HsCIpP.
RD1 refers to the RD value of the ADEP analog at 1 μM as a measure of its ability to activate HsCIpP.
SD refers to the standard deviation of the RD value.
SMILES refers to the structural formulas of the ADEP analogs in SMILES format. Configuration at tetrahedral carbon is specified by @ or @@. Consider the four bonds in the order in which they appear, left to right, in the SMILES form. Looking toward the central carbon from the perspective of the first bond, the other three are either clockwise or counter-clockwise. These cases are indicated with @@ and@, respectively. The structures described by the SMILES format can be drawn using a software like ChemDraw.

### ADEPs Enhance the Peptidase and Protease Activities of HsCIpP

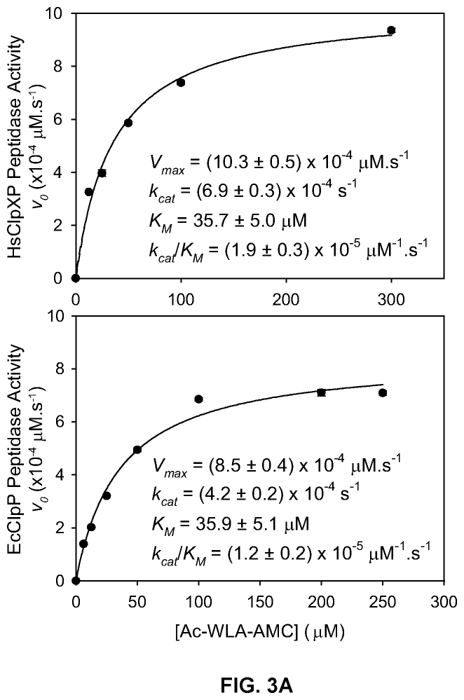

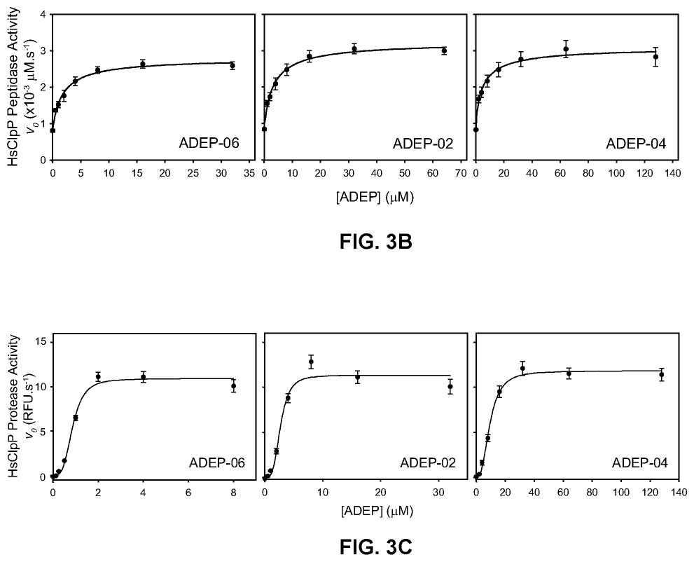

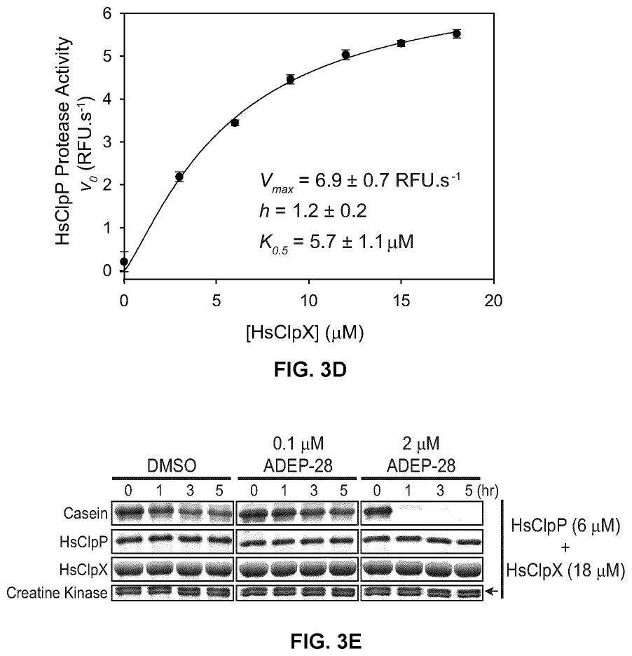

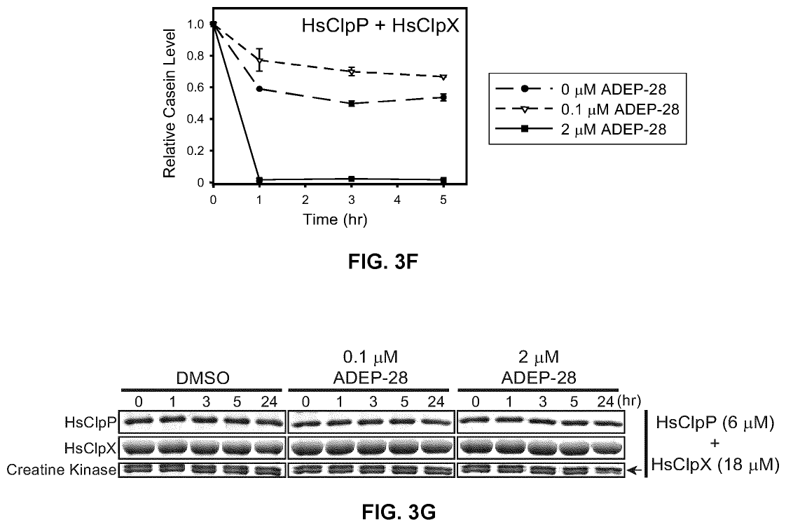

The effects of the ADEPs on the peptidase and protease activities of HsCIpP were investigated using N-acetyl-Trp-Leu-Ala-AMC (Ac-WLA-AMC) and casein-FITC, respectively. Ac-WLA-AMC was the best peptide substrate for HsCIpP among those tested (FIG. 2A). The degradation of Ac-WLA-AMC by 6 μM of HsCIpP (monomeric concentration) has an apparent k_{cat}, K_{M} and k_{cat}/K_{M} lower than that obtained for the degradation of the peptide with 1.5 μM of HsCIpP in the presence of 4.5 μM HsCIpX (compare FIG. 2B with 3A, top panel). Similar values were obtained for the degradation of Ac-WLA-AMC using 2 μM of EcCIpP (FIG. 3A, lower panel). In contrast, while there was no detectable degradation of Suc-LY-AMC by HsCIpP (FIG. 2A), the reported apparent k_{cat} of Suc-LY-AMC degradation by 10 μM EcCIpP (protomer concentration) is 2.4 s⁻¹ with K_{M} of 4.4×10³ μM, yielding a k_{cat}/K_{M} of 5.5×10⁻⁴ s⁻¹.μM⁻¹ (Bewley et al., 2006). Hence, while HsCIpP and EcCIpP have similar general peptidase activities against Ac-WLA-AMC, EcCIpP seems to have broader substrate preferences compared to HsCIpP.

Initial degradation rates data were analyzed using the Hill model. The results for ADEP-28 and ADEP-41 are shown in FIGS. 2C and D, and those for ADEP-06, ADEP-02 and ADEP-04 in FIGS. 3B and C. The kinetic parameters derived are listed in FIG. 2E. With all five ADEP analogs, the peptidase activity of HsCIpP was similarly enhanced, with Vₘₐₓ increased by about 1.8-fold. The Hill coefficients (h), ranging from 0.8 for ADEP-04 to 1.5 for ADEP-41 (FIG. 2E), seem to suggest that the binding of ADEP and the subsequent stimulation of HsCIpP's peptidase activity exhibits only marginal positive cooperativity. The apparent dissociation constants (K_{0.5}) of the ADEP analogs are closely correlated to their respective RD1 scores. Specifically, the higher the RD1 score of a given analog, the lower is its K_{0.5} (FIG. 2E).

Similar to their effects on HsCIpP's peptidase activity, all five ADEP analogs activate and enhance the protease activity of HsCIpP independently of HsCIpX. Notably, the Vₘₐₓ measured in the presence of the five ADEP analogs shows little variation (between 10.9 and 14.0 RFU.s⁻¹; FIG. 2E). Their K_{0.5}, determined by measuring the protease activity of HsCIpP, correlates with their respective RD1 scores. Both ADEP-28 and ADEP-41 exhibit the lowest K_{0.5} (0.12 μM), followed by ADEP-06, ADEP-02, and ADEP-04 (FIG. 2E). In this assay, unlike in the peptidase activity assay, the binding of ADEP and the subsequent activation and enhancement of HsCIpP's protease activity exhibits strong, positive cooperativity. Specifically, enhancement by ADEP-28 has the highest Hill coefficient (FIG. 2E).

By comparison, titration of HsCIpX and fitting the data to the Hill model yields a Vₘₐₓ of 6.88 RFU.s⁻¹, a K_{0.5} of 5.7 μM, and mild positive cooperativity (h=1.25) in HsCIpP-binding (FIG. 3D). These results indicate that the ADEPsexhibit stronger binding to HsCIpP than does HsCIpX under the conditions of these experiments.

### Low Concentration of ADEPs Displace HsCIpX From HsCIpXP Complexes

To assess the effects of ADEPs on the interaction between HsCIpX and HsCIpP, the degradation of casein-FITC by HsCIpXP was monitored in the presence of increasing concentrations of ADEP-28, ADEP-06 or ADEP-02. At low ADEP concentrations, the rate of casein-FITC degradation was decreased, down to an observed minimum (Vₘᵢₙ) that ranges from 0.9 to 1.3 RFU.s⁻¹ (FIGS. 2F and G). Notably, the ADEP concentration needed to minimize the protease activity is dependent on the apparent binding affinity of each analog. As ADEP concentration was further increased, the protease activity increased likely as the population of ADEP-activated HsCIpP increased. Accordingly, the Vₘₐₓ achieved with all three ADEP analogs at saturation is identical to the Vₘₐₓ previously observed in the absence of HsCIpX (FIGS. 2E and F). The initial decrease of protease activity at low ADEP concentrations was not caused by the degradation of HsCIpX, HsCIpP or creatine phosphokinase as shown by SDS-PAGE gels (FIGS. 3E-G).

Taken together, the results clearly illustrate that ADEP can induce the dissociation of the HsCIpXP complex at low concentrations, resulting in a net loss of protease activity. At higher ADEP concentrations, the population of ADEP-activated HsCIpP increases, resulting in the restoration and subsequent increase of protease activity.

### ADEPs Induce HsCIpP-Dependent Cytotoxicity in HEK293 T-REx Cells

To assess the biological impact of ADEPs in targeting HsCIpP in vivo, HEK293 T-REx WT and CLPP^{-/-} cells were treated with serially diluted ADEP-28, ADEP-41, ADEP-06, ADEP-02 and ADEP-04, followed by an assessment of their survival 72 hours post treatment. For HEK293 T-REx WT cells, all five ADEP analogs were found to be cytotoxic with 1050 values between 0.36 μM and 8.20 μM (FIGS. 4A, B and 5A). In comparison, IC₅₀ of cisplatin on HEK293 cells has been reported at 0.43 μM and that of the dihydrofolate reductase-targeting Metoprine at 0.80 μM (Bram et al., 2006).

Importantly, the cytotoxicity of ADEPs showed a strong, linear correlation with their respective apparent binding affinity for HsCIpP (FIG. 4C), revealing a direct link between the potency of ADEPs in activating HsCIpP and their cytotoxicity. Furthermore, ADEP-induced cytotoxicity is HsCIpP-dependent, as CLPP^{-/-} cells, which lack endogenous HsCIpP, are highly resistant to all five ADEP analogs (FIGS. 4A, B and 5A). Hence, without wishing to be bound by theory, these ADEPs are likely acting on-target in cells, although interactions with other proteins that do not cause cytotoxicity cannot yet be ruled out.

It should be noted that, using deep proteomics profiling of mitochondrial extracts isolated from WT and CLPP HEK293 T-REx cells, 281 distinct mitochondrial proteins were identified covering 24% of the estimated human mitochondrial proteome (281 of 1158 human mitochondrial proteins from MitoCarta2.0 inventory (Calvo et al., 2016)). Of these, only 19 mitochondrial proteins in CLPP^{-/-} were significantly (p≤0.05) altered with more than 1.5-fold change compared to WT cells (Table 4). This indicates that there are no drastic changes in the mitochondrial proteome upon CLPP deletion under the conditions tested.

TABLE 4
Relative fold change of mitochondrial proteins identified in the wild type versus CLPP knockouts

| | | | Total peptide ion intensity detected by | | | | | Avg. | |
|---|---|---|---|---|---|---|---|---|---|
| | | | MaxQuant in wild type (WT) and CLPP | | | | | fold | |
| | | | knockout (KO) | | | | | change | |
| UniProt | Protein | Protein | | | CLPP | CLPP | CLPP | (CLPP | P-Value |
| IDs | names | description | WT-1 | WT-2 | KO-1 | KO-2 | KO-3 | KO/WT) | significance |
| Q9UDR5 | AASS | Alpha-aminoadipic | 4.45E+06 | 1.27E+06 | 0.00E+00 | 0.00E+00 | 6.37E+05 | −5.95 | 0.023 |
| | | semialdehyde | | | | | | | |
| | | synthase, | | | | | | | |
| | | mitochondrial; | | | | | | | |
| | | Lysine | | | | | | | |
| | | ketoglutarate | | | | | | | |
| | | reductase; | | | | | | | |
| | | Saccharopine | | | | | | | |
| | | dehydrogenase | | | | | | | |
| Q9NRK6 | ABCB10 | ATP-binding | 6.39E+06 | 5.31E+05 | 0.00E+00 | 9.28E+05 | 0.00E+00 | −6.65 | 0.040 |
| | | cassette sub-family | | | | | | | |
| | | B member 10, | | | | | | | |
| | | mitochondrial | | | | | | | |
| P09110 | ACAA1 | 3-ketoacyl-CoA | 1.18E+06 | 4.49E+05 | 0.00E+00 | 0.00E+00 | 3.83E+04 | −16.43 | 0.018 |
| | | thiolase, | | | | | | | |
| | | peroxisomal | | | | | | | |
| P33121 | ACSL1 | Long-chain-fatty- | 7.15E+05 | 3.90E+05 | 0.00E+00 | 8.11E+03 | 0.00E+00 | −18.71 | 0.042 |
| | | acid--CoA ligase 1 | | | | | | | |
| O14874 | BCKDK | [3-methyl-2- | 8.36E+05 | 0.00E+00 | 5.32E+06 | 4.94E+06 | 9.96E+05 | 20.55 | 0.045 |
| | | oxobutanoate | | | | | | | |
| | | dehydrogenase | | | | | | | |
| | | [lipoamide]] kinase, | | | | | | | |
| | | mitochondrial | | | | | | | |
| Q9HA77 | CARS2 | Probable cysteine-- | 4.64E+06 | 5.63E+05 | 3.55E+06 | 2.73E+06 | 1.92E+06 | 1.97 | 0.045 |
| | | tRNA ligase, | | | | | | | |
| | | mitochondrial | | | | | | | |
| Q16740 | CLPP | ATP-dependent | 4.80E+06 | 1.52E+06 | 0.00E+00 | 0.00E+00 | 0.00E+00 | −86.23 | 0.007 |
| | | Clp protease | | | | | | | |
| | | proteolytic | | | | | | | |
| | | subunit, | | | | | | | |
| | | mitochondrial | | | | | | | |
| P23786 | CPT2 | Carnitine O- | 7.36E+05 | 2.36E+05 | 0.00E+00 | 0.00E+00 | 0.00E+00 | −13.35 | 0.011 |
| | | palmitoyltransferase | | | | | | | |
| | | 2, mitochondrial | | | | | | | |
| Q9BX68 | HINT2 | Histidine triad | 5.14E+06 | 7.84E+05 | 0.00E+00 | 1.29E+06 | 6.01E+05 | −2.66 | 0.038 |
| | | nucleotide-binding | | | | | | | |
| | | protein 2, | | | | | | | |
| | | mitochondrial | | | | | | | |
| Q16891 | IMMT | MICOS | 4.74E+07 | 9.75E+06 | 3.98E+06 | 3.62E+06 | 6.76E+06 | −3.26 | 0.014 |
| | | complex | | | | | | | |
| | | subunit MIC60 | | | | | | | |
| Q9GZY8 | MFF | Mitochondrial | 2.32E+06 | 6.78E+05 | 0.00E+00 | 0.00E+00 | 3.53E+05 | −5.44 | 0.026 |
| | | fission | | | | | | | |
| | | factor | | | | | | | |
| Q9BPX6 | MICU1 | Calcium uptake | 3.14E+06 | 1.22E+06 | 0.00E+00 | 3.18E+05 | 1.35E+05 | −10.74 | 0.022 |
| | | protein 1, | | | | | | | |
| | | mitochondrial | | | | | | | |
| Q8N5N7 | MRPL50 | 39S ribosomal | 7.05E+06 | 2.07E+06 | 0.00E+00 | 1.55E+06 | 6.12E+0 | −4.67 | 0.017 |
| | | protein L50, | | | | | | | |
| | | mitochondrial | | | | | | | |
| Q9BQC6 | MRPL57 | Ribosomal | 2.16E+06 | 7.32E+05 | 0.00E+00 | 0.00E+00 | 1.22E+05 | −13.50 | 0.014 |
| | | protein 63, | | | | | | | |
| | | mitochondrial | | | | | | | |
| Q9Y676 | MRPS18B | 28S ribosomal | 6.24E+06 | 1.56E+06 | 2.00E+05 | 0.00E+00 | 1.03E+06 | −4.54 | 0.030 |
| | | protein S18b, | | | | | | | |
| | | mitochondrial | | | | | | | |
| Q96TC7 | RMDN3 | Regulator of | 6.57E+06 | 3.30E+05 | 4.96E+06 | 3.75E+06 | 2.62E+06 | 2.51 | 0.049 |
| | | microtubule | | | | | | | |
| | | dynamics | | | | | | | |
| | | protein 3 | | | | | | | |
| Q99595 | TIMM17A | Mitochondrial | 1.18E+06 | 5.56E+05 | 0.00E+00 | 0.00E+00 | 0.00E+00 | −27.78 | 0.030 |
| | | import | | | | | | | |
| | | inner membrane | | | | | | | |
| | | translocase | | | | | | | |
| | | subunit | | | | | | | |
| | | Tim17-A | | | | | | | |
| Q9Y5J7 | TIMM9 | Mitochondrial | 3.79E+06 | 4.82E+05 | 0.00E+00 | 0.00E+00 | 6.70E+04 | −21.32 | 0.003 |
| | | import | | | | | | | |
| | | inner membrane | | | | | | | |
| | | translocase subunit | | | | | | | |
| | | Tim9 | | | | | | | |
| P49411 | TUFM | Elongation | 1.19E+08 | 1.65E+07 | 1.77E+07 | 2.86E+07 | 1.45E+07 | −1.90 | 0.015 |
| | | factor Tu, | | | | | | | |
| | | mitochondrial | | | | | | | |
| Q5JTZ9 | AARS2 | Alanine--tRNA | 1.36E+07 | 7.20E+05 | 8.12E+05 | 2.44E+06 | 5.95E+05 | −2.75 | 0.119 |
| | | ligase, | | | | | | | |
| | | mitochondrial | | | | | | | |
| P11310 | ACADM | Medium-chain | 1.46E+07 | 1.26E+06 | 2.59E+07 | 1.15E+07 | 8.28E+06 | 3.88 | 0.083 |
| | | specific acyl-CoA | | | | | | | |
| | | dehydrogenase, | | | | | | | |
| | | mitochondrial | | | | | | | |
| P53396 | ACLY | ATP-citrate | 1.82E+06 | 6.51E+05 | 0.00E+00 | 5.69E+05 | 6.62E+05 | −1.96 | 0.173 |
| | | synthase | | | | | | | |
| Q4G176 | ACSF3 | Acyl-CoA | 1.20E+06 | 6.33E+05 | 0.00E+00 | 0.00E+00 | 5.83E+05 | −2.65 | 0.177 |
| | | synthetase | | | | | | | |
| | | family member 3, | | | | | | | |
| | | mitochondrial | | | | | | | |
| O95573; | ACSL3; | Long-chain-fatty- | 6.15E+06 | 0.00E+00 | 3.15E+06 | 0.00E+00 | 1.55E+06 | 1.57 | 0.341 |
| O60488 | ACSL4 | acid--CoA ligase 3; | | | | | | | |
| | | Long-chain-fatty- | | | | | | | |
| | | acid--CoA ligase 4 | | | | | | | |
| Q9Y4W6 | AFG3L2 | AFG3-like | 7.33E+06 | 1.34E+06 | 1.07E+05 | 2.91E+06 | 1.55E+06 | −1.72 | 0.161 |
| | | protein 2 | | | | | | | |
| P27144 | AK4 | Adenylate kinase 4, | 7.31E+06 | 5.38E+06 | 4.51E+06 | 4.08E+06 | 1.75E+06 | −2.01 | 0.175 |
| | | mitochondrial | | | | | | | |
| P30837 | ALDH1B1 | Aldehyde | 3.29E+07 | 1.18E+07 | 5.95E+05 | 1.87E+07 | 5.88E+06 | −2.26 | 0.114 |
| | | dehydrogenase X, | | | | | | | |
| | | mitochondrial | | | | | | | |
| P05091 | ALDH2 | Aldehyde | 2.77E+07 | 8.69E+06 | 0.00E+00 | 4.29E+06 | 8.41E+06 | −2.53 | 0.120 |
| | | dehydrogenase, | | | | | | | |
| | | mitochondrial | | | | | | | |
| P51648 | ALDH3A2 | Fatty aldehyde | 2.87E+06 | 1.52E+06 | 0.00E+00 | 2.01E+06 | 1.46E+06 | −1.61 | 0.269 |
| | | dehydrogenase | | | | | | | |
| Q02252 | ALDH6Al | Methylmalonate- | 1.06E+07 | 2.54E+06 | 0.00E+00 | 1.92E+06 | 2.46E+06 | −2.55 | 0.081 |
| | | semialdehyde | | | | | | | |
| | | dehydrogenase | | | | | | | |
| | | [acylating], | | | | | | | |
| | | mitochondrial | | | | | | | |
| P78540 | ARG2 | Arginase-2, | 2.30E+06 | 4.54E+05 | 0.00E+00 | 0.00E+00 | 3.33E+06 | 1.99 | 0.361 |
| | | mitochondrial | | | | | | | |
| Q5T9A4 | ATAD3B | ATPase family AAA | 3.85E+07 | 1.12E+07 | 0.00E+00 | 5.14E+06 | 1.35E+07 | −2.21 | 0.170 |
| | | domain-containing | | | | | | | |
| | | protein 3B | | | | | | | |
| P25705 | ATP5A1 | ATP synthase | 3.29E+08 | 2.62E+07 | 2.45E+08 | 9.28E+07 | 6.85E+07 | 1.57 | 0.263 |
| | | subunit alpha, | | | | | | | |
| | | mitochondrial | | | | | | | |
| P36542 | ATP5C1 | ATP synthase | 2.14E+07 | 1.09E+07 | 8.61E+06 | 1.97E+07 | 2.17E+06 | −1.70 | 0.215 |
| | | subunit gamma, | | | | | | | |
| | | mitochondrial | | | | | | | |
| P30049 | ATP5D | ATP synthase | 2.20E+06 | 8.62E+05 | 1.09E+06 | 5.80E+06 | 8.91E+05 | 1.78 | 0.265 |
| | | subunit delta, | | | | | | | |
| | | mitochondrial | | | | | | | |
| P56134 | ATP5J2 | ATP synthase | 1.57E+06 | 7.44E+05 | 2.94E+05 | 6.54E+05 | 8.33E+05 | −1.50 | 0.277 |
| | | subunit f, | | | | | | | |
| | | mitochondrial | | | | | | | |
| O75964 | ATP5L | ATP synthase | 3.65E+06 | 8.98E+05 | 0.00E+00 | 0.00E+00 | 8.39E+05 | −3.45 | 0.073 |
| | | subunit g, | | | | | | | |
| | | mitochondrial | | | | | | | |
| Q9Y276 | BCS1L | Mitochondrial | 1.51E+06 | 5.30E+04 | 0.00E+00 | 0.00E+00 | 4.62E+04 | −7.41 | 0.095 |
| | | chaperone | | | | | | | |
| | | BCS1 | | | | | | | |
| Q86WA6 | BPHL | Valacyclovir | 1.61E+06 | 1.22E+05 | 4.32E+06 | 3.16E+06 | 6.60E+04 | 5.23 | 0.162 |
| | | hydrolase | | | | | | | |
| Q99832 | CCT7 | T-complex protein 1 | 2.85E+07 | 0.00E+00 | 5.91E+04 | 0.00E+00 | 1.38E+04 | −189.33 | 0.136 |
| | | subunit eta | | | | | | | |
| Q9NYJ1 | COA4 | Cytochrome c | 3.80E+06 | 1.80E+06 | 1.94E+06 | 0.00E+00 | 1.03E+06 | −2.17 | 0.154 |
| | | oxidase assembly | | | | | | | |
| | | factor 4 homolog, | | | | | | | |
| | | mitochondrial | | | | | | | |
| P10606 | COX5B | Cytochrome c | 1.64E+07 | 3.20E+06 | 0.00E+00 | 0.00E+00 | 4.73E+06 | −2.52 | 0.160 |
| | | oxidase subunit 5B, | | | | | | | |
| | | mitochondrial | | | | | | | |
| P12074 | COX6A1 | Cytochrome c | 2.54E+07 | 8.04E+06 | 8.52E+05 | 1.02E+08 | 1.25E+07 | 2.54 | 0.278 |
| | | oxidase subunit 6A1, | | | | | | | |
| | | mitochondrial | | | | | | | |
| P14854 | COX6B1 | Cytochrome c | 7.85E+06 | 5.56E+06 | 6.04E+06 | 5.29E+06 | 1.11E+06 | −1.84 | 0.208 |
| | | oxidase subunit 6B1 | | | | | | | |
| P09669 | COX6C | Cytochrome c | 4.46E+06 | 1.99E+05 | 0.00E+00 | 0.00E+00 | 0.00E+00 | −36.69 | 0.061 |
| | | oxidase subunit 6C | | | | | | | |
| O75390 | CS | Citrate synthase, | 1.15E+08 | 7.45E+06 | 1.01E+08 | 3.56E+07 | 1.93E+07 | 1.78 | 0.262 |
| | | mitochondrial | | | | | | | |
| P51398 | DAP3 | 28S ribosomal | 7.97E+06 | 6.12E+06 | 0.00E+00 | 1.81E+06 | 5.85E+06 | −2.17 | 0.238 |
| | | protein S29, | | | | | | | |
| | | mitochondrial | | | | | | | |
| P11182 | DBT | Lipoamide | 0.00E+00 | 1.80E+05 | 1.27E+06 | 0.00E+00 | 3.91E+04 | 1.96 | 0.357 |
| | | acyltransferase | | | | | | | |
| | | component of | | | | | | | |
| | | branched-chain | | | | | | | |
| | | alpha-keto acid | | | | | | | |
| | | dehydrogenase | | | | | | | |
| | | complex, | | | | | | | |
| | | mitochondrial | | | | | | | |
| P09622 | DLD | Dihydrolipoyl | 1.19E+07 | 6.40E+05 | 6.51E+05 | 1.27E+06 | 0.00E+00 | −5.34 | 0.077 |
| | | dehydrogenase, | | | | | | | |
| | | mitochondrial | | | | | | | |
| Q9NVH1 | DNAJC11 | DnaJ homolog | 9.16E+05 | 6.32E+05 | 0.00E+00 | 4.66E+06 | 7.46E+05 | 1.96 | 0.305 |
| | | subfamily C | | | | | | | |
| | | member 11 | | | | | | | |
| O00429 | DNM1L | Dynamin-1-like | 1.09E+06 | 5.14E+05 | 0.00E+00 | 0.00E+00 | 3.31E+05 | −3.72 | 0.097 |
| | | protein | | | | | | | |
| P30084 | ECHS1 | Enoyl-CoA | 1.38E+08 | 6.41E+06 | 1.14E+07 | 2.39E+07 | 7.50E+06 | −2.37 | 0.147 |
| | | hydratase, | | | | | | | |
| | | mitochondrial | | | | | | | |
| O75521 | ECI2 | Enoyl-CoA delta | 2.71E+06 | 1.45E+06 | 0.00E+00 | 0.00E+00 | 7.36E+05 | −4.83 | 0.084 |
| | | isomerase 2, | | | | | | | |
| | | mitochondrial | | | | | | | |
| Q96GK7; | FAHD2A; | Fumarylacetoacetate | 1.32E+06 | 4.59E+05 | 0.00E+00 | 0.00E+00 | 2.77E+05 | −4.28 | 0.053 |
| Q6P2I3 | FAHD2B | hydrolase domain- | | | | | | | |
| | | containing protein 2A; | | | | | | | |
| | | Fumarylacetoacetate | | | | | | | |
| | | hydrolase | | | | | | | |
| | | domain-containing | | | | | | | |
| | | protein 2B | | | | | | | |
| P49327 | FASN | Fatty acid | 1.91E+07 | 2.54E+06 | 3.49E+06 | 2.64E+07 | 4.88E+06 | 1.68 | 0.282 |
| | | synthase; | | | | | | | |
| | | [Acyl-carrier-protein] | | | | | | | |
| | | S-acetyltransferase; | | | | | | | |
| | | [Acyl-carrier-protein] | | | | | | | |
| | | S-malonyltransferase; | | | | | | | |
| | | 3-oxoacyl-[acyl- | | | | | | | |
| | | carrier-protein] | | | | | | | |
| | | synthase; | | | | | | | |
| | | 3-oxoacyl- | | | | | | | |
| | | [acyl-carrier-protein] | | | | | | | |
| | | reductase; | | | | | | | |
| | | 3-hydroxyacyl-[acyl- | | | | | | | |
| | | carrier-protein] | | | | | | | |
| | | dehydratase; | | | | | | | |
| | | Enoyl- | | | | | | | |
| | | [acyl-carrier-protein] | | | | | | | |
| | | reductase; | | | | | | | |
| | | Oleoyl- | | | | | | | |
| | | [acyl-carrier-protein] | | | | | | | |
| | | hydrolase | | | | | | | |
| P10109 | FDX1 | Adrenodoxin, | 6.94E+06 | 3.01E+06 | 0.00E+00 | 0.00E+00 | 4.54E+06 | −1.81 | 0.301 |
| | | mitochondrial | | | | | | | |
| P22830 | FECH | Ferrochelatase, | 2.73E+06 | 1.80E+06 | 1.48E+06 | 1.15E+07 | 1.37E+06 | 1.75 | 0.308 |
| | | mitochondrial | | | | | | | |
| Q14318 | FKBP8 | Peptidyl-prolyl | 1.74E+07 | 2.54E+06 | 1.46E+07 | 1.06E+07 | 6.63E+06 | 1.86 | 0.058 |
| | | cis-trans | | | | | | | |
| | | isomerase | | | | | | | |
| | | FKBP8 | | | | | | | |
| Q8TAE8 | GADD4 | Growth arrest and | 1.16E+06 | 2.23E+06 | 0.00E+00 | 9.57E+05 | 1.57E+06 | −2.29 | 0.246 |
| | 5G1P1 | DNA damage- | | | | | | | |
| | | inducible proteins- | | | | | | | |
| | | interacting protein 1 | | | | | | | |
| P04406 | GAPDH | Glyceraldehyde-3- | 1.45E+07 | 8.03E+05 | 7.34E+06 | 3.20E+07 | 5.87E+06 | 3.84 | 0.127 |
| | | phosphate | | | | | | | |
| | | dehydrogenase | | | | | | | |
| O43716 | GATC | Glutamyl-tRNA(Gln) | 1.01E+06 | 4.77E+05 | 0.00E+00 | 4.62E+06 | 1.26E+06 | 2.75 | 0.217 |
| | | amidotransferase | | | | | | | |
| | | subunit C, | | | | | | | |
| | | mitochondrial | | | | | | | |
| O75323 | GBAS | Protein NipSnap | 1.20E+07 | 2.38E+06 | 0.00E+00 | 9.87E+05 | 3.37E+06 | −2.28 | 0.154 |
| | | homolog 2 | | | | | | | |
| Q96RP9 | GFM1 | Elongation factor G, | 1.67E+06 | 2.76E+05 | 0.00E+00 | 4.10E+06 | 3.82E+05 | 2.11 | 0.312 |
| | | mitochondrial | | | | | | | |
| P32189; | GK; GK3P | Glycerol kinase; | 1.00E+06 | 7.78E+05 | 0.00E+00 | 2.46E+06 | 0.00E+00 | −1.51 | 0.370 |
| Q14409 | | Putative glycerol | | | | | | | |
| | | kinase 3 | | | | | | | |
| P00367; | GLUD1; | Glutamate | 6.67E+07 | 1.95E+07 | 6.48E+06 | 2.39E+07 | 2.29E+07 | −1.60 | 0.202 |
| P49448 | GLUD2 | dehydrogenase 1, | | | | | | | |
| | | mitochondrial; | | | | | | | |
| | | Glutamate | | | | | | | |
| | | dehydrogenase 2, | | | | | | | |
| | | mitochondrial | | | | | | | |
| P00505 | GOT2 | Aspartate | 2.79E+06 | 1.29E+06 | 0.00E+00 | 1.72E+06 | 9.15E+05 | −1.96 | 0.176 |
| | | aminotransferase, | | | | | | | |
| | | mitochondrial | | | | | | | |
| Q12849 | GRSF1 | G-rich | 7.52E+05 | 5.11E+05 | 3.61E+06 | 2.31E+06 | 0.00E+00 | 2.65 | 0.234 |
| | | sequence | | | | | | | |
| | | factor 1 | | | | | | | |
| Q16836 | HADH | Hydroxyacyl- | 7.75E+06 | 3.92E+06 | 2.87E+06 | 7.33E+06 | 1.48E+06 | −1.53 | 0.236 |
| | | coenzyme A | | | | | | | |
| | | dehydrogenase, | | | | | | | |
| | | mitochondrial | | | | | | | |
| P55084 | HADHB | Trifunctional enzyme | 1.22E+07 | 2.44E+06 | 3.60E+06 | 2.38E+07 | 3.51E+06 | 1.89 | 0.261 |
| | | subunit beta, | | | | | | | |
| | | mitochondrial; | | | | | | | |
| | | 3-ketoacyl-CoA | | | | | | | |
| | | thiolase | | | | | | | |
| Q9Y241 | HIGD1A | HIG1 domain | 2.35E+07 | 4.99E+06 | 1.17E+06 | 2.87E+06 | 5.27E+06 | −2.44 | 0.077 |
| | | family member 1A, | | | | | | | |
| | | mitochondrial | | | | | | | |
| P19367 | HK1 | Hexokinase-1 | 6.33E+06 | 1.00E+06 | 0.00E+00 | 3.52E+06 | 5.19E+05 | −1.86 | 0.193 |
| Q99714 | HSD17B10 | 3-hydroxyacyl-CoA | 6.22E+07 | 1.44E+07 | 3.69E+07 | 1.10E+08 | 1.95E+07 | 1.90 | 0.179 |
| | | dehydrogenase | | | | | | | |
| | | type-2 | | | | | | | |
| P51659 | HSD17B4 | Peroxisomal | 1.13E+07 | 4.14E+06 | 8.99E+05 | 4.36E+06 | 3.55E+06 | −1.94 | 0.141 |
| | | multifunctional | | | | | | | |
| | | enzyme type 2; | | | | | | | |
| | | (3R)-hydroxyacyl- | | | | | | | |
| | | CoA dehydrogenase; | | | | | | | |
| | | Enoyl-CoA | | | | | | | |
| | | hydratase 2 | | | | | | | |
| Q9NSE4 | IARS2 | Isoleucine--tRNA | 9.19E+06 | 2.71E+05 | 0.00E+00 | 2.31E+06 | 8.82E+05 | −1.87 | 0.258 |
| | | ligase, mitochondrial | | | | | | | |
| P50213 | IDH3A | Isocitrate | 8.80E+06 | 4.38E+05 | 0.00E+00 | 4.07E+06 | 6.56E+05 | −1.50 | 0.332 |
| | | dehydrogenase | | | | | | | |
| | | [NAD] subunit alpha, | | | | | | | |
| | | mitochondrial | | | | | | | |
| Q96AB3 | ISOC2 | Isochorismatase | 1.36E+06 | 7.22E+05 | 0.00E+00 | 1.78E+06 | 1.51E+05 | −1.80 | 0.268 |
| | | domain-containing | | | | | | | |
| | | protein 2, | | | | | | | |
| | | mitochondrial | | | | | | | |
| Q96GW9 | MARS2 | Methionine--tRNA | 2.25E+05 | 1.16E+05 | 0.00E+00 | 2.94E+05 | 0.00E+00 | −1.59 | 0.294 |
| | | ligase, mitochondrial | | | | | | | |
| Q8IVS2 | MCAT | Malonyl-CoA-acyl | 1.10E+06 | 1.06E+06 | 0.00E+00 | 2.30E+06 | 4.52E+05 | −1.52 | 0.330 |
| | | carrier protein | | | | | | | |
| | | transacylase, | | | | | | | |
| | | mitochondrial | | | | | | | |
| Q96RQ3 | MCCC1 | Methylcrotonoyl- | 2.45E+06 | 1.13E+06 | 0.00E+00 | 1.92E+06 | 4.37E+05 | −2.19 | 0.159 |
| | | CoA | | | | | | | |
| | | carboxylase subunit | | | | | | | |
| | | alpha, mitochondrial | | | | | | | |
| P23368 | ME2 | NAD-dependent | 4.23E+07 | 3.64E+06 | 1.20E+07 | 8.22E+06 | 2.31E+06 | −1.63 | 0.191 |
| | | malic enzyme, | | | | | | | |
| | | mitochondrial | | | | | | | |
| Q8IYU8 | MICU2 | Calcium uptake | 3.15E+06 | 2.92E+05 | 0.00E+00 | 4.08E+05 | 7.57E+05 | −1.70 | 0.258 |
| | | protein 2, | | | | | | | |
| | | mitochondrial | | | | | | | |
| Q9Y3B7 | MRPL11 | 39S ribosomal | 2.41E+07 | 7.95E+06 | 2.80E+06 | 3.14E+06 | 9.26E+06 | −1.91 | 0.189 |
| | | protein L11, | | | | | | | |
| | | mitochondrial | | | | | | | |
| P52815 | MRPL12 | 39S ribosomal | 4.41E+07 | 2.75E+06 | 4.46E+07 | 3.30E+07 | 7.43E+06 | 2.41 | 0.149 |
| | | protein L12, | | | | | | | |
| | | mitochondrial | | | | | | | |
| Q9BYD1 | MRPL13 | 39S ribosomal | 5.94E+06 | 9.42E+05 | 1.24E+07 | 4.94E+06 | 1.89E+06 | 3.01 | 0.172 |
| | | protein L13, | | | | | | | |
| | | mitochondrial | | | | | | | |
| Q9P015 | MRPL15 | 39S ribosomal | 5.88E+06 | 1.16E+06 | 2.49E+06 | 6.85E+06 | 1.81E+06 | 1.53 | 0.181 |
| | | protein L15, | | | | | | | |
| | | mitochondrial | | | | | | | |
| Q9NRX2 | MRPL17 | 39S ribosomal | 1.53E+06 | 3.70E+05 | 0.00E+00 | 2.25E+06 | 2.15E+06 | 2.59 | 0.230 |
| | | protein L17, | | | | | | | |
| | | mitochondrial | | | | | | | |
| Q9H0U6 | MRPL18 | 39S ribosomal | 2.98E+06 | 3.05E+05 | 0.00E+00 | 0.00E+00 | 7.78E+05 | −2.02 | 0.241 |
| | | protein L18, | | | | | | | |
| | | mitochondrial | | | | | | | |
| P49406 | MRPL19 | 39S ribosomal | 3.64E+06 | 9.49E+05 | 6.42E+05 | 7.74E+06 | 1.00E+06 | 1.64 | 0.326 |
| | | protein L19, | | | | | | | |
| | | mitochondrial | | | | | | | |
| Q16540 | MRPL23 | 39S ribosomal | 2.00E+06 | 2.71E+06 | 9.38E+05 | 1.00E+06 | 1.77E+06 | −2.10 | 0.233 |
| | | protein L23, | | | | | | | |
| | | mitochondrial | | | | | | | |
| Q96A35 | MRPL24 | 39S ribosomal | 2.04E+06 | 1.63E+06 | 0.00E+00 | 0.00E+00 | 1.62E+06 | −2.32 | 0.240 |
| | | protein L24, | | | | | | | |
| | | mitochondrial | | | | | | | |
| Q9P0M9 | MRPL27 | 39S ribosomal | 1.13E+06 | 2.78E+06 | 0.00E+00 | 0.00E+00 | 1.68E+06 | −3.36 | 0.207 |
| | | protein L27, | | | | | | | |
| | | mitochondrial | | | | | | | |
| Q96DV4 | MRPL38 | 39S ribosomal | 1.53E+06 | 8.48E+05 | 0.00E+00 | 1.23E+06 | 2.98E+05 | −2.35 | 0.155 |
| | | protein L38, | | | | | | | |
| | | mitochondrial | | | | | | | |
| Q9NYK5 | MRPL39 | 39S ribosomal | 7.18E+06 | 2.35E+06 | 1.34E+07 | 7.98E+06 | 5.73E+06 | 2.51 | 0.064 |
| | | protein L39, | | | | | | | |
| | | mitochondrial | | | | | | | |
| Q9BYD3 | MRPL4 | 39S ribosomal | 6.93E+06 | 1.70E+06 | 9.44E+06 | 8.48E+06 | 1.80E+06 | 2.03 | 0.140 |
| | | protein L4, | | | | | | | |
| | | mitochondrial | | | | | | | |
| Q8IXM3 | MRPL41 | 39S ribosomal | 3.73E+06 | 3.92E+05 | 7.86E+06 | 1.28E+07 | 5.69E+05 | 5.52 | 0.122 |
| | | protein L41, | | | | | | | |
| | | mitochondrial | | | | | | | |
| Q8N983 | MRPL43 | 39S ribosomal | 4.40E+06 | 9.42E+04 | 7.32E+06 | 1.57E+06 | 1.12E+06 | 3.62 | 0.200 |
| | | protein L43, | | | | | | | |
| | | mitochondrial | | | | | | | |
| Q9H2W6 | MRPL46 | 39S ribosomal | 5.90E+06 | 3.16E+06 | 0.00E+00 | 0.00E+00 | 1.08E+06 | −7.30 | 0.060 |
| | | protein L46, | | | | | | | |
| | | mitochondrial | | | | | | | |
| Q96EL3 | MRPL53 | 39S ribosomal | 2.21E+06 | 5.90E+05 | 0.00E+00 | 1.33E+06 | 4.44E+05 | −1.72 | 0.177 |
| | | protein L53, | | | | | | | |
| | | mitochondrial | | | | | | | |
| O60783 | MRPS14 | 28S ribosomal | 3.75E+06 | 2.49E+06 | 2.60E+06 | 1.41E+06 | 1.84E+06 | −1.49 | 0.268 |
| | | protein S14, | | | | | | | |
| | | mitochondrial | | | | | | | |
| Q9Y3D93 | MRPS2 | 28S ribosomal | 8.27E+06 | 2.42E+06 | 2.33E+06 | 1.30E+07 | 4.17E+06 | 1.56 | 0.251 |
| | | protein S23, | | | | | | | |
| | | mitochondrial | | | | | | | |
| Q9BYN86 | MRPS2 | 28S ribosomal | 1.97E+06 | 5.25E+05 | 8.97E+06 | 2.58E+06 | 1.07E+06 | 4.54 | 0.178 |
| | | protein S26, | | | | | | | |
| | | mitochondrial | | | | | | | |
| Q9Y2Q98 | MRPS2 | 28S ribosomal | 3.25E+06 | 1.88E+06 | 6.17E+05 | 0.00E+00 | 1.60E+06 | −2.41 | 0.182 |
| | | protein S28, | | | | | | | |
| | | mitochondrial | | | | | | | |
| Q926651 | MRPS3 | 28S ribosomal | 4.51E+06 | 9.43E+05 | 6.41E+05 | 5.99E+05 | 1.13E+06 | −1.90 | 0.108 |
| | | protein S31, | | | | | | | |
| | | mitochondrial | | | | | | | |
| Q9Y2913 | MRPS3 | 28S ribosomal | 1.27E+07 | 5.08E+05 | 0.00E+00 | 0.00E+00 | 4.99E+05 | −10.23 | 0.082 |
| | | protein S33, | | | | | | | |
| | | mitochondrial | | | | | | | |
| P829304 | MRPS3 | 28S ribosomal | 1.20E+07 | 3.59E+06 | 1.82E+07 | 1.13E+07 | 3.93E+06 | 1.83 | 0.182 |
| | | protein S34, | | | | | | | |
| | | mitochondrial | | | | | | | |
| P82932 | MRPS6 | 28S ribosomal | 5.47E+06 | 9.09E+05 | 1.24E+06 | 0.00E+00 | 1.25E+06 | −1.84 | 0.159 |
| | | protein S6, | | | | | | | |
| | | mitochondrial | | | | | | | |
| Q9Y2R9 | MRPS7 | 28S ribosomal | 6.44E+06 | 2.04E+05 | 0.00E+00 | 6.43E+05 | 1.73E+05 | −5.18 | 0.116 |
| | | protein S7, | | | | | | | |
| | | mitochondrial | | | | | | | |
| P00403 | MT-CO2 | Cytochrome c | 7.20E+06 | 2.34E+06 | 0.00E+00 | 7.56E+05 | 2.80E+06 | −2.22 | 0.184 |
| | | oxidase subunit 2 | | | | | | | |
| P11586 | MTHFD1 | C-1-tetrahydrofolate | 2.18E+06 | 4.41E+05 | 0.00E+00 | 1.97E+06 | 0.00E+00 | −1.64 | 0.316 |
| | | synthase, | | | | | | | |
| | | cytoplasmic; | | | | | | | |
| | | Methyl- | | | | | | | |
| | | enetetrahydrofolate | | | | | | | |
| | | dehydrogenase; | | | | | | | |
| | | Methenyl- | | | | | | | |
| | | tetrahydrofolate | | | | | | | |
| | | cyclohydrolase; | | | | | | | |
| | | Formyl- | | | | | | | |
| | | tetrahydrofolate | | | | | | | |
| | | synthetase; | | | | | | | |
| | | C-1-tetrahydrofolate | | | | | | | |
| | | synthase, | | | | | | | |
| | | cytoplasmic, | | | | | | | |
| | | N-terminally | | | | | | | |
| | | processed | | | | | | | |
| P13995 | MTHFD2 | Bifunctional | 2.60E+06 | 9.36E+05 | 0.00E+00 | 3.52E+06 | 7.12E+06 | 3.24 | 0.257 |
| | | methylenetetra- | | | | | | | |
| | | hydrofolate | | | | | | | |
| | | dehydrogenase/ | | | | | | | |
| | | cyclohydrolase, | | | | | | | |
| | | mitochondrial; | | | | | | | |
| | | NAD-dependent | | | | | | | |
| | | methylenetetra- | | | | | | | |
| | | hydrofolate | | | | | | | |
| | | dehydrogenase; | | | | | | | |
| | | Methenyl- | | | | | | | |
| | | tetrahydrofolate | | | | | | | |
| | | cyclohydrolase | | | | | | | |
| Q13505 | MTX1 | Metaxin-1 | 1.70E+06 | 1.00E+06 | 0.00E+00 | 0.00E+00 | 6.15E+05 | −3.88 | 0.115 |
| O75431 | MTX2 | Metaxin-2 | 2.47E+06 | 0.00E+00 | 1.37E+06 | 4.21E+06 | 1.14E+05 | 3.29 | 0.212 |
| O95299 | NDUFA10 | NADH | 3.03E+06 | 1.47E+05 | 9.30E+06 | 3.12E+06 | 1.17E+06 | 6.13 | 0.152 |
| | | dehydrogenase | | | | | | | |
| | | [ubiquinone] 1 alpha | | | | | | | |
| | | subcomplex | | | | | | | |
| | | subunit 10, | | | | | | | |
| | | mitochondrial | | | | | | | |
| Q9P0J0 | NDUFA13 | NADH | 3.42E+06 | 1.08E+06 | 2.03E+06 | 1.19E+07 | 1.82E+06 | 2.65 | 0.196 |
| | | dehydrogenase | | | | | | | |
| | | [ubiquinone] 1 alpha | | | | | | | |
| | | subcomplex | | | | | | | |
| | | subunit 13 | | | | | | | |
| P51970 | NDUFA8 | NADH | 3.69E+06 | 8.74E+05 | 0.00E+00 | 1.98E+06 | 3.89E+05 | −2.18 | 0.113 |
| | | dehydrogenase | | | | | | | |
| | | [ubiquinone] 1 alpha | | | | | | | |
| | | subcomplex | | | | | | | |
| | | subunit 8 | | | | | | | |
| Q16795 | NDUFA9 | NADH | 5.96E+06 | 1.74E+06 | 0.00E+00 | 3.65E+06 | 1.18E+06 | −1.84 | 0.165 |
| | | dehydrogenase | | | | | | | |
| | | [ubiquinone] 1 alpha | | | | | | | |
| | | subcomplex | | | | | | | |
| | | subunit 9, | | | | | | | |
| | | mitochondrial | | | | | | | |
| Q8N183 | NDUFAF2 | Mimitin, | 1.03E+07 | 1.25E+06 | 0.00E+00 | 1.43E+06 | 1.62E+06 | −2.59 | 0.080 |
| | | mitochondrial | | | | | | | |
| P28331 | NDUFS1 | NADH-ubiquinone | 2.19E+07 | 4.62E+06 | 6.80E+05 | 8.69E+06 | 2.35E+06 | −2.41 | 0.053 |
| | | oxidoreductase | | | | | | | |
| | | 75 kDa | | | | | | | |
| | | subunit, | | | | | | | |
| | | mitochondrial | | | | | | | |
| O75306 | NDUFS2 | NADH | 2.54E+06 | 8.48E+05 | 8.05E+06 | 2.24E+06 | 9.31E+05 | 2.73 | 0.226 |
| | | dehydrogenase | | | | | | | |
| | | [ubiquinone] iron- | | | | | | | |
| | | sulfur protein 2, | | | | | | | |
| | | mitochondrial | | | | | | | |
| O00217 | NDUFS8 | NADH | 2.95E+06 | 8.49E+05 | 0.00E+00 | 1.51E+06 | 4.39E+04 | −3.34 | 0.076 |
| | | dehydrogenase | | | | | | | |
| | | [ubiquinone] iron- | | | | | | | |
| | | sulfur protein 8, | | | | | | | |
| | | mitochondrial | | | | | | | |
| Q9BPW8 | NIPSNAP1 | Protein NipSnap | 9.70E+06 | 5.63E+06 | 1.08E+06 | 9.00E+05 | 3.40E+06 | −3.19 | 0.118 |
| | | homolog 1 | | | | | | | |
| Q9H857 | NT5DC2 | 5-nucleotidase | 2.48E+05 | 2.09E+05 | 0.00E+00 | 7.48E+05 | 8.23E+05 | 2.48 | 0.242 |
| | | domain-containing | | | | | | | |
| | | protein 2 | | | | | | | |
| Q02218 | OGDH | 2-oxoglutarate | 7.59E+06 | 7.92E+05 | 1.74E+06 | 3.27E+05 | 1.07E+06 | −1.85 | 0.138 |
| | | dehydrogenase, | | | | | | | |
| | | mitochondrial | | | | | | | |
| Q9Y3D7 | PAM16 | Mitochondrial | 1.91E+06 | 1.15E+06 | 7.76E+05 | 0.00E+00 | 1.37E+06 | −1.57 | 0.312 |
| | | import inner | | | | | | | |
| | | membrane | | | | | | | |
| | | translocase subunit | | | | | | | |
| | | TIM16 | | | | | | | |
| P05166 | PCCB | Propionyl-CoA | 8.76E+05 | 5.67E+05 | 0.00E+00 | 0.00E+00 | 0.00E+00 | −26.16 | 0.052 |
| | | carboxylase | | | | | | | |
| | | beta chain, | | | | | | | |
| | | mitochondrial | | | | | | | |
| Q16822 | PCK2 | Phospho- | 3.33E+06 | 1.48E+06 | 0.00E+00 | 0.00E+00 | 7.47E+05 | −5.13 | 0.064 |
| | | enolpyruvate | | | | | | | |
| | | carboxykinase | | | | | | | |
| | | [GTP], | | | | | | | |
| | | mitochondrial | | | | | | | |
| Q6L8Q7 | PDE12 | 2,5- | 2.06E+06 | 6.39E+05 | 0.00E+00 | 1.01E+06 | 0.00E+00 | −3.68 | 0.067 |
| | | phosphodiesterase | | | | | | | |
| | | 12 | | | | | | | |
| O00330 | PDHX | Pyruvate | 1.76E+06 | 1.18E+06 | 0.00E+00 | 1.80E+06 | 3.32E+05 | −2.36 | 0.179 |
| | | dehydrogenase | | | | | | | |
| | | protein X | | | | | | | |
| | | component, | | | | | | | |
| | | mitochondrial | | | | | | | |
| Q96HS1 | PGAM5 | Serine/threonine- | 1.36E+07 | 1.88E+05 | 0.00E+00 | 0.00E+00 | 2.59E+06 | −1.97 | 0.312 |
| | | protein phosphatase | | | | | | | |
| | | PGAM5, | | | | | | | |
| | | mitochondrial | | | | | | | |
| P35232 | PHB | Prohibitin | 2.35E+08 | 8.14E+07 | 3.00E+08 | 2.80E+08 | 5.31E+07 | 1.54 | 0.234 |
| Q5JRX3 | PITRM1 | Presequence | 9.57E+06 | 2.02E+06 | 0.00E+00 | 1.62E+07 | 3.49E+06 | 1.55 | 0.338 |
| | | protease, | | | | | | | |
| | | mitochondrial | | | | | | | |
| Q10713 | PMPCA | Mitochondrial- | 6.70E+06 | 1.21E+06 | 0.00E+00 | 5.46E+05 | 1.45E+06 | −2.71 | 0.088 |
| | | processing | | | | | | | |
| | | peptidase subunit | | | | | | | |
| | | alpha | | | | | | | |
| O75439 | PMPCB | Mitochondrial- | 2.26E+07 | 8.76E+05 | 2.89E+06 | 4.00E+07 | 3.06E+06 | 2.47 | 0.283 |
| | | processing | | | | | | | |
| | | peptidase subunit | | | | | | | |
| | | beta | | | | | | | |
| Q8TCS8 | PNPT1 | Polyribonucleotide | 1.23E+07 | 4.46E+05 | 3.78E+05 | 2.75E+05 | 2.99E+06 | −1.56 | 0.351 |
| | | nucleotidyl- | | | | | | | |
| | | transferase 1, | | | | | | | |
| | | mitochondrial | | | | | | | |
| Q9Y2S7 | POLDIP2 | Polymerase delta- | 8.87E+06 | 4.93E+05 | 1.41E+07 | 1.97E+07 | 2.00E+06 | 4.89 | 0.092 |
| | | interacting protein 2 | | | | | | | |
| O00411 | POLRMT | DNA-directed | 2.60E+06 | 0.00E+00 | 1.47E+06 | 4.81E+05 | 5.76E+05 | 1.79 | 0.253 |
| | | RNA polymerase, | | | | | | | |
| | | mitochondrial | | | | | | | |
| P32119 | PRDX2 | Peroxiredoxin-2 | 1.64E+06 | 9.81E+05 | 0.00E+00 | 0.00E+00 | 2.30E+05 | −9.17 | 0.059 |
| Q13162 | PRDX4 | Peroxiredoxin-4 | 2.34E+06 | 9.28E+05 | 0.00E+00 | 0.00E+00 | 1.02E+06 | −2.49 | 0.182 |
| P30044 | PRDX5 | Peroxiredoxin-5, | 1.03E+07 | 3.34E+05 | 1.24E+07 | 3.31E+06 | 1.09E+06 | 2.38 | 0.264 |
| | | mitochondrial | | | | | | | |
| Q9H7Z7 | PTGES2 | Prostaglandin E | 3.90E+05 | 4.93E+05 | 0.00E+00 | 8.96E+05 | 0.00E+00 | −2.27 | 0.251 |
| | | synthase 2; | | | | | | | |
| | | Prostaglandin E | | | | | | | |
| | | synthase 2 | | | | | | | |
| | | truncated form | | | | | | | |
| Q96C36 | PYCR2 | Pyrroline-5- | 1.55E+07 | 7.07E+05 | 0.00E+00 | 0.00E+00 | 2.46E+06 | −2.75 | 0.197 |
| | | carboxylate | | | | | | | |
| | | reductase 2 | | | | | | | |
| Q9HOR6 | QRSL1 | Glutamyl-tRNA(Gln) | 1.84E+06 | 7.62E+05 | 0.00E+00 | 0.00E+00 | 8.28E+05 | −2.45 | 0.187 |
| | | amidotransferase | | | | | | | |
| | | subunit A, | | | | | | | |
| | | mitochondrial | | | | | | | |
| Q8IX12 | RHOT1 | Mitochondrial Rho | 1.09E+06 | 6.62E+05 | 0.00E+00 | 0.00E+00 | 4.88E+05 | −3.16 | 0.147 |
| | | GTPase 1 | | | | | | | |
| P62906 | RPL10A | 60S ribosomal | 1.55E+07 | 3.36E+06 | 0.00E+00 | 7.71E+06 | 3.25E+06 | −1.72 | 0.175 |
| | | protein L10a | | | | | | | |
| P62263 | RPS14 | 40S ribosomal | 3.29E+07 | 1.15E+07 | 0.00E+00 | 4.77E+06 | 7.50E+06 | −3.44 | 0.065 |
| | | protein S14 | | | | | | | |
| P62244 | RPS15A | 40S ribosomal | 3.39E+06 | 7.27E+05 | 0.00E+00 | 5.95E+05 | 1.06E+06 | −1.89 | 0.191 |
| | | protein S15a | | | | | | | |
| P62269 | RPS18 | 40S ribosomal | 1.19E+07 | 5.80E+06 | 1.17E+06 | 2.63E+06 | 1.79E+06 | −4.09 | 0.058 |
| | | protein S18 | | | | | | | |
| Q6P087 | RPUSD3 | RNA | 1.34E+06 | 7.03E+05 | 0.00E+00 | 3.32E+05 | 3.30E+05 | −3.49 | 0.086 |
| | | pseudouridylate | | | | | | | |
| | | synthase domain- | | | | | | | |
| | | containing protein 3 | | | | | | | |
| Q9NP81 | SARS2 | Serine--tRNA ligase, | 1.95E+07 | 1.90E+06 | 0.00E+00 | 1.03E+07 | 1.83E+06 | −1.56 | 0.288 |
| | | mitochondrial | | | | | | | |
| P31040 | SDHA | Succinate | 1.37E+06 | 8.56E+05 | 1.44E+07 | 2.43E+06 | 1.74E+06 | 5.48 | 0.195 |
| | | dehydrogenase | | | | | | | |
| | | [ubiquinone] | | | | | | | |
| | | flavoprotein subunit, | | | | | | | |
| | | mitochondrial | | | | | | | |
| P21912 | SDHB | Succinate | 1.05E+07 | 1.28E+06 | 4.22E+05 | 1.85E+07 | 1.37E+06 | 1.70 | 0.354 |
| | | dehydrogenase | | | | | | | |
| | | [ubiquinone] iron- | | | | | | | |
| | | sulfur subunit, | | | | | | | |
| | | mitochondrial | | | | | | | |
| P53007 | SLC25Al | Tricarboxylate | 2.18E+06 | 4.50E+05 | 0.00E+00 | 6.21E+06 | 1.22E+06 | 2.58 | 0.246 |
| | | transport protein, | | | | | | | |
| | | mitochondrial | | | | | | | |
| O75746 | SLC25A12 | Calcium-binding | 1.51E+06 | 8.50E+05 | 0.00E+00 | 6.19E+05 | 3.85E+05 | −3.03 | 0.105 |
| | | mitochondrial carrier | | | | | | | |
| | | protein Aralar1 | | | | | | | |
| P05141 | SLC25A5 | ADP/ATP | 1.05E+08 | 8.05E+06 | 1.13E+08 | 5.38E+07 | 2.75E+07 | 2.31 | 0.155 |
| | | translocase 2; | | | | | | | |
| | | ADP/ATP | | | | | | | |
| | | translocase 2, N- | | | | | | | |
| | | terminally processed | | | | | | | |
| P12236 | SLC25A6 | ADP/ATP | 2.00E+07 | 2.11E+06 | 3.42E+07 | 4.48E+06 | 1.16E+07 | 3.10 | 0.173 |
| | | translocase 3; | | | | | | | |
| | | ADP/ATP | | | | | | | |
| | | translocase 3, N- | | | | | | | |
| | | terminally processed | | | | | | | |
| Q9GZT3 | SLIRP | SRA stem-loop- | 1.78E+07 | 2.97E+06 | 0.00E+00 | 6.31E+06 | 1.60E+06 | −2.58 | 0.058 |
| | | interacting RNA- | | | | | | | |
| | | binding protein, | | | | | | | |
| | | mitochondrial | | | | | | | |
| Q8IYB8 | SUPV3L1 | ATP-dependent | 5.14E+06 | 8.42E+05 | 0.00E+00 | 6.92E+06 | 2.06E+06 | 1.57 | 0.317 |
| | | RNA helicase | | | | | | | |
| | | SUPV3L1, | | | | | | | |
| | | mitochondrial | | | | | | | |
| Q9BSH4 | TACO1 | Translational | 8.87E+06 | 1.33E+06 | 0.00E+00 | 1.71E+06 | 1.44E+06 | −2.50 | 0.063 |
| | | activator of | | | | | | | |
| | | cytochrome c | | | | | | | |
| | | oxidase 1 | | | | | | | |
| Q9BW92 | TARS2 | Threonine-- | 6.82E+06 | 3.42E+06 | 0.00E+00 | 5.41E+06 | 2.21E+06 | −1.86 | 0.205 |
| | | tRNA ligase, | | | | | | | |
| | | mitochondrial | | | | | | | |
| Q9Y5L4 | TIMM13 | Mitochondrial | 4.55E+06 | 5.90E+05 | 0.00E+00 | 1.23E+06 | 9.94E+05 | −1.70 | 0.195 |
| | | import inner | | | | | | | |
| | | membrane | | | | | | | |
| | | translocase subunit | | | | | | | |
| | | Tim13 | | | | | | | |
| O43615 | T1MM44 | Mitochondrial | 2.22E+07 | 9.31E+05 | 0.00E+00 | 3.69E+06 | 1.25E+06 | −3.17 | 0.137 |
| | | import inner | | | | | | | |
| | | membrane | | | | | | | |
| | | translocase subunit | | | | | | | |
| | | TIM44 | | | | | | | |
| O60220 | TIMM8A | Mitochondrial | 2.17E+07 | 1.01E+07 | 6.06E+06 | 9.25E+06 | 6.92E+06 | −1.81 | 0.146 |
| | | import inner | | | | | | | |
| | | membrane | | | | | | | |
| | | translocase subunit | | | | | | | |
| | | Tim8 A | | | | | | | |
| Q15388 | TOMM20 | Mitochondrial | 2.23E+06 | 7.19E+05 | 0.00E+00 | 1.40E+07 | 1.46E+06 | 3.78 | 0.250 |
| | | import receptor | | | | | | | |
| | | subunit TOM20 | | | | | | | |
| | | homolog | | | | | | | |
| O94826 | TOMM70A | Mitochondrial | 2.82E+07 | 1.35E+06 | 0.00E+00 | 1.65E+06 | 1.30E+06 | −5.96 | 0.081 |
| | | import receptor | | | | | | | |
| | | subunit | | | | | | | |
| | | TOM70 | | | | | | | |
| P14927 | UQCRB | Cytochrome b-c1 | 8.50E+05 | 5.87E+05 | 0.00E+00 | 0.00E+00 | 5.33E+04 | −15.37 | 0.060 |
| | | complex subunit 7 | | | | | | | |
| P31930 | UQCRC1 | Cytochrome b-c1 | 1.10E+07 | 1.85E+06 | 0.00E+00 | 5.34E+07 | 3.59E+06 | 3.92 | 0.266 |
| | | complex subunit 1, | | | | | | | |
| | | mitochondrial | | | | | | | |
| P22695 | UQCRC2 | Cytochrome b-c1 | 1.17E+07 | 2.88E+06 | 0.00E+00 | 2.63E+07 | 1.13E+07 | 2.47 | 0.213 |
| | | complex subunit 2, | | | | | | | |
| | | mitochondrial | | | | | | | |
| P45880 | VDAC2 | Voltage-dependent | 2.62E+07 | 4.16E+06 | 9.80E+04 | 1.92E+06 | 5.46E+06 | −2.69 | 0.097 |
| | | anion-selective | | | | | | | |
| | | channel protein 2 | | | | | | | |
| P40939 | HADHA | Trifunctional enzyme | 3.95E+07 | 9.56E+06 | 2.59E+07 | 3.16E+07 | 1.43E+07 | 1.40 | 0.020 |
| | | subunit alpha, | | | | | | | |
| | | mitochondrial; | | | | | | | |
| | | Long-chain | | | | | | | |
| | | enoyl-CoA | | | | | | | |
| | | hydratase; | | | | | | | |
| | | Long chain | | | | | | | |
| | | 3-hydroxyacyl- | | | | | | | |
| | | CoA | | | | | | | |
| | | dehydrogenase | | | | | | | |
| P04181 | OAT | Ornithine | 1.08E+08 | 2.95E+07 | 7.16E+07 | 9.65E+07 | 4.92E+07 | 1.46 | 0.031 |
| | | aminotransferase, | | | | | | | |
| | | mitochondrial; | | | | | | | |
| | | Ornithine | | | | | | | |
| | | aminotransferase, | | | | | | | |
| | | hepatic form; | | | | | | | |
| | | Ornithine | | | | | | | |
| | | aminotransferase, | | | | | | | |
| | | renal form | | | | | | | |
| P42765 | ACAA2 | 3-ketoacyl-CoA | 4.37E+07 | 9.50E+06 | 6.95E+06 | 3.21E+07 | 7.06E+06 | −1.27 | 0.313 |
| | | thiolase, | | | | | | | |
| | | mitochondrial | | | | | | | |
| P45954 | ACADSB | Short/branched | 3.24E+06 | 1.98E+06 | 0.00E+00 | 4.39E+06 | 2.50E+06 | −1.07 | 0.466 |
| | | chain specific | | | | | | | |
| | | acyl-CoA | | | | | | | |
| | | dehydrogenase, | | | | | | | |
| | | mitochondrial | | | | | | | |
| P24752 | ACAT1 | Acetyl-CoA | 1.32E+08 | 2.41E+07 | 2.07E+07 | 6.41E+07 | 5.41E+07 | 1.04 | 0.469 |
| | | acetyltransferase, | | | | | | | |
| | | mitochondrial | | | | | | | |
| Q99798 | ACO2 | Aconitate hydratase, | 4.15E+07 | 1.88E+06 | 0.00E+00 | 9.92E+06 | 1.27E+07 | −1.05 | 0.481 |
| | | mitochondrial | | | | | | | |
| O95831 | AIFM1 | Apoptosis-inducing | 3.14E+07 | 4.18E+06 | 3.29E+06 | 1.10E+07 | 5.73E+06 | −1.48 | 0.155 |
| | | factor 1, | | | | | | | |
| | | mitochondrial | | | | | | | |
| | | Adenylate kinase 2, | | | | | | | |
| | | mitochondrial; | | | | | | | |
| P54819 | AK2 | Adenylate | 2.88E+07 | 7.81E+06 | 1.32E+07 | 1.71E+07 | 1.19E+07 | 1.11 | 0.332 |
| | | kinase 2, | | | | | | | |
| | | mitochondrial, | | | | | | | |
| | | N-terminally | | | | | | | |
| | | processed | | | | | | | |
| Q92667 | AKAP1 | A-kinase anchor | 2.97E+06 | 3.92E+05 | 0.00E+00 | 2.32E+06 | 2.83E+05 | −1.27 | 0.393 |
| | | protein 1, | | | | | | | |
| | | mitochondrial | | | | | | | |
| P54886 | ALDH18A1 | Delta-1-pyrroline- | 1.35E+08 | 2.60E+07 | 5.16E+07 | 5.80E+07 | 3.60E+07 | −1.03 | 0.383 |
| | | 5-carboxylate | | | | | | | |
| | | synthase; | | | | | | | |
| | | Glutamate | | | | | | | |
| | | 5-kinase; | | | | | | | |
| | | Gamma- | | | | | | | |
| | | glutamyl phosphate | | | | | | | |
| | | reductase | | | | | | | |
| P30038 | ALDH4A1 | Delta-1-pyrroline- | 6.19E+05 | 1.09E+05 | 0.00E+00 | 1.55E+05 | 1.95E+05 | −1.41 | 0.274 |
| | | 5-carboxylate | | | | | | | |
| | | dehydrogenase, | | | | | | | |
| | | mitochondrial | | | | | | | |
| P49419 | ALDH7A1 | Alpha-aminoadipic | 7.80E+06 | 9.70E+05 | 0.00E+00 | 8.35E+06 | 1.01E+06 | 1.09 | 0.468 |
| | | semialdehyde | | | | | | | |
| | | dehydrogenase | | | | | | | |
| Q9NVI7 | ATAD3A | ATPase family | 6.21E+07 | 2.29E+07 | 6.86E+06 | 9.35E+07 | 2.58E+07 | 1.13 | 0.434 |
| | | AAAd omain- | | | | | | | |
| | | containing | | | | | | | |
| | | protein 3A | | | | | | | |
| P06576 | ATP5B | ATP synthase | 9.49E+08 | 2.13E+08 | 7.81E+08 | 5.50E+08 | 3.42E+08 | 1.45 | 0.134 |
| | | subunit beta, | | | | | | | |
| | | mitochondrial | | | | | | | |
| P24539 | ATP5F1 | ATP synthase F(0) | 3.56E+06 | 7.58E+04 | 3.21E+06 | 0.00E+00 | 0.00E+00 | 1.40 | 0.423 |
| | | complex subunit B1, | | | | | | | |
| | | mitochondrial | | | | | | | |
| O75947 | ATP5H | ATP synthase | 3.25E+07 | 1.23E+07 | 1.99E+07 | 3.12E+07 | 1.11E+07 | 1.09 | 0.389 |
| | | subunit d, | | | | | | | |
| | | mitochondrial | | | | | | | |
| P18859 | ATP5J | ATP synthase- | 1.16E+07 | 4.58E+06 | 9.35E+06 | 5.64E+06 | 4.57E+06 | 1.00 | 0.497 |
| | | coupling factor 6, | | | | | | | |
| | | mitochondrial | | | | | | | |
| P48047 | ATP5O | ATP synthase | 6.83E+07 | 5.94E+06 | 1.47E+07 | 1.62E+07 | 1.52E+07 | −1.12 | 0.397 |
| | | subunit O, | | | | | | | |
| | | mitochondrial | | | | | | | |
| Q07021 | C1QBP | Complement | 2.76E+07 | 1.58E+07 | 4.00E+07 | 3.36E+07 | 7.57E+06 | 1.19 | 0.385 |
| | | component 1 Q | | | | | | | |
| | | subcomponent- | | | | | | | |
| | | binding protein, | | | | | | | |
| | | mitochondrial | | | | | | | |
| Q4VC31 | CCDC58 | Coiled-coil | 3.81E+06 | 3.22E+06 | 4.47E+05 | 1.02E+07 | 2.88E+06 | 1.07 | 0.471 |
| | | domain-containing | | | | | | | |
| | | protein 58 | | | | | | | |
| Q9BXW7 | CECR5 | Cat eye | 7.73E+06 | 2.46E+06 | 5.92E+05 | 7.56E+06 | 3.11E+06 | −1.06 | 0.465 |
| | | syndrome | | | | | | | |
| | | critical region | | | | | | | |
| | | protein 5 | | | | | | | |
| P12532 | CKMT1A | Creatine kinase | 1.94E+07 | 8.62E+05 | 9.48E+05 | 9.54E+06 | 8.08E+05 | −1.44 | 0.355 |
| | | U-type, | | | | | | | |
| | | mitochondrial | | | | | | | |
| O76031 | CLPX | ATP-dependent | 9.68E+06 | 2.00E+06 | 1.73E+06 | 4.26E+06 | 3.57E+06 | −1.09 | 0.419 |
| | | CIp protease | | | | | | | |
| | | ATP-binding | | | | | | | |
| | | subunit cIpX-like, | | | | | | | |
| | | mitochondrial | | | | | | | |
| Q5HYK3 | COQ5 | 2-methoxy-6- | 9.71E+05 | 4.62E+05 | 0.00E+00 | 1.29E+05 | 9.38E+05 | −1.19 | 0.440 |
| | | polyprenyl-1,4- | | | | | | | |
| | | benzoquinol | | | | | | | |
| | | methylase, | | | | | | | |
| | | mitochondrial | | | | | | | |
| P20674 | COX5A | Cytochrome c | 1.07E+08 | 5.62E+06 | 6.44E+06 | 5.78E+07 | 1.15E+07 | −1.13 | 0.440 |
| | | oxidase subunit 5A, | | | | | | | |
| | | mitochondrial | | | | | | | |
| P00387 | CYB5R3 | NADH-cytochrome | 5.74E+06 | 4.61E+06 | 9.35E+06 | 1.35E+04 | 3.40E+06 | −1.20 | 0.419 |
| | | b5 reductase 3; | | | | | | | |
| | | NADH-cytochrome | | | | | | | |
| | | b5 reductase 3 | | | | | | | |
| | | membrane-bound | | | | | | | |
| | | form; NADH- | | | | | | | |
| | | cytochrome b5 | | | | | | | |
| | | reductase 3 | | | | | | | |
| | | soluble form | | | | | | | |
| P08574 | CYC1 | Cytochrome c1, | 1.39E+06 | 1.87E+06 | 0.00E+00 | 9.24E+06 | 9.12E+05 | 1.32 | 0.417 |
| | | heme protein, | | | | | | | |
| | | mitochondrial | | | | | | | |
| Q6PI48 | DARS2 | Aspartate--tRNA | 1.21E+07 | 2.71E+06 | 9.29E+05 | 1.29E+07 | 2.21E+06 | −1.07 | 0.466 |
| | | ligase, | | | | | | | |
| | | mitochondrial | | | | | | | |
| Q9NUL7 | DDX28 | Probable ATP- | 1.09E+06 | 4.84E+05 | 0.00E+00 | 0.00E+00 | 9.29E+05 | −1.36 | 0.397 |
| | | dependent RNA | | | | | | | |
| | | helicase DDX28 | | | | | | | |
| Q7L2E3 | DHX30 | Putative ATP- | 1.44E+07 | 7.43E+06 | 1.61E+06 | 2.05E+07 | 5.50E+06 | −1.19 | 0.406 |
| | | dependent RNA | | | | | | | |
| | | helicase DHX30 | | | | | | | |
| P10515 | DLAT | Dihydrolipoyllysine- | 1.23E+07 | 3.62E+06 | 7.53E+06 | 1.29E+06 | 2.73E+06 | −1.43 | 0.265 |
| | | residue | | | | | | | |
| | | acetyltransferase | | | | | | | |
| | | component of | | | | | | | |
| | | pyruvate | | | | | | | |
| | | dehydrogenase | | | | | | | |
| | | complex, | | | | | | | |
| | | mitochondrial | | | | | | | |
| P36957 | DLST | Dihydrolipoyllysine- | 3.89E+06 | 6.08E+05 | 6.98E+05 | 2.72E+06 | 2.01E+06 | 1.46 | 0.277 |
| | | residue | | | | | | | |
| | | succinyltransferase | | | | | | | |
| | | component of 2- | | | | | | | |
| | | oxoglutarate | | | | | | | |
| | | dehydrogenase | | | | | | | |
| | | complex, | | | | | | | |
| | | mitochondrial | | | | | | | |
| Q13011 | ECH1 | Delta(3,5)- | 2.88E+07 | 5.35E+06 | 1.17E+07 | 2.35E+07 | 8.92E+06 | 1.33 | 0.121 |
| | | Delta(2,4)-dienoyl- | | | | | | | |
| | | CoA isomerase, | | | | | | | |
| | | mitochondrial | | | | | | | |
| P13804 | ETFA | Electron transfer | 3.78E+07 | 6.40E+06 | 2.29E+07 | 1.30E+07 | 7.21E+06 | 1.06 | 0.436 |
| | | flavoprotein subunit | | | | | | | |
| | | alpha, mitochondrial | | | | | | | |
| P38117 | ETFB | Electron transfer | 2.68E+07 | 3.43E+06 | 1.75E+06 | 7.77E+06 | 6.23E+06 | −1.46 | 0.234 |
| | | flavoprotein subunit | | | | | | | |
| | | beta | | | | | | | |
| P22570 | FDXR | NADPH: | 2.58E+06 | 6.77E+05 | 0.00E+00 | 2.57E+06 | 6.19E+05 | −1.17 | 0.413 |
| | | adrenodoxin | | | | | | | |
| | | oxidoreductase, | | | | | | | |
| | | mitochondrial | | | | | | | |
| P07954 | FH | Fumarate hydratase, | 1.18E+07 | 2.88E+06 | 0.00E+00 | 7.11E+06 | 3.56E+06 | −1.39 | 0.294 |
| | | mitochondrial | | | | | | | |
| Q92947 | GCDH | Glutaryl-CoA | 3.64E+06 | 4.55E+05 | 0.00E+00 | 2.71E+06 | 1.19E+06 | 1.16 | 0.419 |
| | | dehydrogenase, | | | | | | | |
| | | mitochondrial | | | | | | | |
| Q86SX6 | GLRX5 | Glutaredox | 9.15E+05 | 3.26E+05 | 0.00E+00 | 2.81E+06 | 0.00E+00 | 1.46 | 0.407 |
| | | in-related | | | | | | | |
| | | protein 5, | | | | | | | |
| | | mitochondrial | | | | | | | |
| P43304 | GPD2 | Glycerol-3- | 5.37E+06 | 4.68E+05 | 0.00E+00 | 2.76E+06 | 7.83E+05 | −1.31 | 0.354 |
| | | phosphate | | | | | | | |
| | | dehydrogenase, | | | | | | | |
| | | mitochondrial | | | | | | | |
| Q9HAV7 | GRPEL1 | GrpE protein | 1.12E+07 | 9.67E+05 | 0.00E+00 | 1.16E+07 | 1.45E+06 | 1.21 | 0.433 |
| | | homolog 1, | | | | | | | |
| | | mitochondrial | | | | | | | |
| Q9Y2Q3 | GSTK1 | Glutathione S- | 3.17E+06 | 5.02E+05 | 0.00E+00 | 4.52E+06 | 4.90E+05 | 1.29 | 0.414 |
| | | transferase kappa 1 | | | | | | | |
| P31937 | HIBADH | 3-hydroxyisobutyrate | 2.71E+06 | 6.14E+05 | 0.00E+00 | 3.84E+06 | 3.56E+05 | 1.04 | 0.486 |
| | | dehydrogenase, | | | | | | | |
| | | mitochondrial | | | | | | | |
| P38646 | HSPA9 | Stress-70 protein, | 3.15E+08 | 7.14E+07 | 1.28E+08 | 2.33E+08 | 7.98E+07 | 1.08 | 0.352 |
| | | mitochondrial | | | | | | | |
| P10809 | HSPD1 | 60 kDa heat shock | 8.21E+08 | 2.84E+08 | 7.93E+08 | 5.91E+08 | 2.47E+08 | 1.21 | 0.324 |
| | | protein, | | | | | | | |
| | | mitochondrial | | | | | | | |
| P61604 | HSPE1 | 10 kDa heat shock | 3.83E+07 | 9.74E+06 | 9.67E+06 | 3.12E+07 | 7.61E+06 | −1.14 | 0.378 |
| | | protein, | | | | | | | |
| | | mitochondrial | | | | | | | |
| Q5T440 | IBA57 | Putative transferase | 3.15E+06 | 4.47E+05 | 0.00E+00 | 0.00E+00 | 1.48E+06 | −1.31 | 0.411 |
| | | CAF17, | | | | | | | |
| | | mitochondrial | | | | | | | |
| P48735 | 1DH2 | Isocitrate | 2.83E+06 | 6.13E+05 | 0.00E+00 | 2.06E+06 | 7.53E+05 | −1.23 | 0.369 |
| | | dehydrogenase | | | | | | | |
| | | [NADP], | | | | | | | |
| | | mitochondrial | | | | | | | |
| O43837 | IDH3B | Isocitrate | 1.81E+07 | 1.61E+06 | 2.04E+06 | 1.58E+07 | 5.96E+05 | −1.01 | 0.496 |
| | | dehydrogenase | | | | | | | |
| | | [NAD] subunit beta, | | | | | | | |
| | | mitochondrial | | | | | | | |
| P51553 | IDH3G | Isocitrate | 3.24E+06 | 9.38E+05 | 0.00E+00 | 7.33E+06 | 7.25E+05 | 1.45 | 0.386 |
| | | dehydrogenase | | | | | | | |
| | | [NAD] subunit | | | | | | | |
| | | gamma, | | | | | | | |
| | | mitochondrial | | | | | | | |
| P28838 | LAP3 | Cytosol | 8.29E+06 | 2.49E+06 | 0.00E+00 | 8.39E+06 | 2.74E+06 | −1.14 | 0.427 |
| | | aminopeptidase | | | | | | | |
| P07195 | LDHB | L-lactate | 1.33E+07 | 3.58E+06 | 4.06E+06 | 1.19E+07 | 2.42E+06 | −1.10 | 0.416 |
| | | dehydrogenase B | | | | | | | |
| | | chain | | | | | | | |
| O95202 | LETM1 | LETM1 and | 2.01E+07 | 5.77E+06 | 3.69E+06 | 1.84E+07 | 6.62E+06 | −1.03 | 0.475 |
| | | EF-hand | | | | | | | |
| | | domain-containing | | | | | | | |
| | | protein 1, | | | | | | | |
| | | mitochondrial | | | | | | | |
| P36776 | LONP1 | Lon protease | 5.65E+07 | 1.22E+07 | 2.47E+07 | 2.87E+07 | 7.23E+06 | −1.21 | 0.245 |
| | | homolog, | | | | | | | |
| | | mitochondrial | | | | | | | |
| P42704 | LRPPRC | Leucine-rich PPR | 6.52E+07 | 1.49E+07 | 1.28E+07 | 3.50E+07 | 1.57E+07 | −1.29 | 0.196 |
| | | motif-containing | | | | | | | |
| | | protein, | | | | | | | |
| | | mitochondrial | | | | | | | |
| Q9HCCO | MCCC2 | Methylcrotonoyl- | 1.16E+07 | 2.81E+06 | 5.86E+06 | 5.46E+06 | 2.78E+06 | −1.06 | 0.363 |
| | | CoA carboxylase | | | | | | | |
| | | beta chain, | | | | | | | |
| | | mitochondrial | | | | | | | |
| P40926 | MDH2 | Malate | 5.44E+07 | 1.79E+07 | 2.76E+07 | 1.83E+07 | 1.10E+07 | −1.47 | 0.163 |
| | | dehydrogenase, | | | | | | | |
| | | mitochondrial | | | | | | | |
| Q8IWA4 | MFN1 | Mitofusin-1 | 3.58E+05 | 6.80E+04 | 0.00E+00 | 3.40E+05 | 0.00E+00 | −1.21 | 0.409 |
| Q99797 | MIPEP | Mitochondrial | 7.26E+06 | 4.53E+05 | 0.00E+00 | 5.42E+06 | 1.22E+06 | 1.12 | 0.454 |
| | | intermediate | | | | | | | |
| | | peptidase | | | | | | | |
| Q5T653 | MRPL2 | 39S ribosomal | 1.18E+07 | 3.84E+06 | 6.20E+06 | 3.71E+06 | 2.29E+06 | −1.48 | 0.171 |
| | | protein L2, | | | | | | | |
| | | mitochondrial | | | | | | | |
| Q9BYC9 | MRPL20 | 39S ribosomal | 3.84E+06 | 1.29E+06 | 0.00E+00 | 4.71E+06 | 4.53E+05 | −1.40 | 0.358 |
| | | protein L20, | | | | | | | |
| | | mitochondrial | | | | | | | |
| Q9NWU5 | MRPL22 | 39S ribosomal | 6.61E+06 | 2.91E+06 | 6.36E+06 | 6.58E+06 | 1.79E+06 | 1.11 | 0.408 |
| | | protein L22, | | | | | | | |
| | | mitochondrial | | | | | | | |
| P09001 | MRPL3 | 39S ribosomal | 2.39E+06 | 1.24E+06 | 0.00E+00 | 6.21E+06 | 1.14E+06 | 1.28 | 0.407 |
| | | protein L3, | | | | | | | |
| | | mitochondrial | | | | | | | |
| Q9BZE1 | MRPL37 | 39S ribosomal | 7.58E+06 | 2.51E+06 | 0.00E+00 | 3.59E+06 | 4.50E+06 | −1.21 | 0.412 |
| | | protein L37, | | | | | | | |
| | | mitochondrial | | | | | | | |
| Q9NQ50 | MRPL40 | 39S ribosomal | 2.03E+06 | 2.25E+05 | 0.00E+00 | 2.07E+06 | 4.24E+05 | 1.26 | 0.402 |
| | | protein L40, | | | | | | | |
| | | mitochondrial | | | | | | | |
| Q9H9J2 | MRPL44 | 39S ribosomal | 1.07E+06 | 5.50E+05 | 0.00E+00 | 2.87E+06 | 5.80E+05 | 1.38 | 0.376 |
| | | protein L44, | | | | | | | |
| | | mitochondrial | | | | | | | |
| Q9BRJ2 | MRPL45 | 39S ribosomal | 1.07E+07 | 7.85E+06 | 6.12E+06 | 1.44E+07 | 5.03E+06 | −1.21 | 0.374 |
| | | protein L45, | | | | | | | |
| | | mitochondrial | | | | | | | |
| Q9HD33 | MRPL47 | 39S ribosomal | 3.51E+06 | 1.12E+06 | 0.00E+00 | 6.29E+06 | 4.30E+05 | 1.03 | 0.491 |
| | | protein L47, | | | | | | | |
| | | mitochondrial | | | | | | | |
| Q86TS9 | MRPL52 | 39S ribosomal | 4.12E+06 | 2.08E+06 | 0.00E+00 | 5.97E+06 | 1.73E+06 | −1.18 | 0.417 |
| | | protein L52, | | | | | | | |
| | | mitochondrial | | | | | | | |
| Q7Z7F7 | MRPL55 | 39S ribosomal | 2.55E+06 | 1.49E+06 | 5.06E+05 | 1.94E+06 | 2.58E+06 | 1.01 | 0.496 |
| | | protein L55, | | | | | | | |
| | | mitochondrial | | | | | | | |
| Q9NP92 | MRPS30 | 28S ribosomal | 4.48E+06 | 0.00E+00 | 1.13E+06 | 1.39E+06 | 8.40E+04 | −1.12 | 0.454 |
| | | protein S30, | | | | | | | |
| | | mitochondrial | | | | | | | |
| P82673 | MRPS35 | 28S ribosomal | 6.01E+06 | 1.17E+06 | 9.11E+05 | 7.73E+06 | 1.44E+05 | 1.00 | 0.499 |
| | | protein S35, | | | | | | | |
| | | mitochondrial | | | | | | | |
| P82675 | MRPS5 | 28S ribosomal | 7.28E+05 | 3.80E+05 | 9.49E+05 | 6.88E+05 | 2.17E+05 | 1.14 | 0.410 |
| | | protein S5, | | | | | | | |
| | | mitochondrial | | | | | | | |
| P82933 | MRPS9 | 28S ribosomal | 2.53E+06 | 4.89E+05 | 0.00E+00 | 1.92E+06 | 2.94E+05 | −1.47 | 0.302 |
| | | protein S9, | | | | | | | |
| | | mitochondrial | | | | | | | |
| | | Monofunctional | | | | | | | |
| Q6UB35 | MTHFD1L | C1-tetrahydrofolate | 1.67E+07 | 5.02E+06 | 4.90E+05 | 8.33E+06 | 7.60E+06 | −1.31 | 0.348 |
| | | synthase, | | | | | | | |
| | | mitochondrial | | | | | | | |
| P22033 | MUT | Methylmalonyl- | 2.10E+06 | 9.02E+05 | 0.00E+00 | 2.52E+06 | 1.08E+06 | −1.07 | 0.464 |
| | | CoA mutase, | | | | | | | |
| | | mitochondrial | | | | | | | |
| O43678 | NDUFA2 | NADH | 1.74E+07 | 3.45E+06 | 2.73E+06 | 1.15E+07 | 3.55E+06 | −1.19 | 0.331 |
| | | dehydrogenase | | | | | | | |
| | | [ubiquinone] 1 alpha | | | | | | | |
| | | subcomplex | | | | | | | |
| | | subunit 2 | | | | | | | |
| Q16718 | NDUFA5 | NADH | 1.62E+07 | 2.94E+06 | 9.87E+06 | 4.25E+06 | 3.28E+06 | −1.01 | 0.493 |
| | | dehydrogenase | | | | | | | |
| | | [ubiquinone] 1 alpha | | | | | | | |
| | | subcomplex | | | | | | | |
| | | subunit 5 | | | | | | | |
| Q9P032 | NDUFAF4 | NADH | 6.24E+06 | 2.59E+06 | 4.51E+06 | 2.24E+06 | 1.82E+06 | −1.28 | 0.307 |
| | | dehydrogenase | | | | | | | |
| | | [ubiquinone] 1 alpha | | | | | | | |
| | | subcomplex | | | | | | | |
| | | assembly | | | | | | | |
| | | factor 4 | | | | | | | |
| O95168 | NDUFB4 | NADH | 5.38E+06 | 9.26E+05 | 0.00E+00 | 4.67E+06 | 1.47E+06 | 1.01 | 0.493 |
| | | dehydrogenase | | | | | | | |
| | | [ubiquinone] 1 beta | | | | | | | |
| | | subcomplex | | | | | | | |
| | | subunit 4 | | | | | | | |
| O95139 | NDUFB6 | NADH | 9.62E+06 | 1.14E+06 | 5.29E+06 | 0.00E+00 | 1.80E+06 | −1.12 | 0.442 |
| | | dehydrogenase | | | | | | | |
| | | [ubiquinone] 1 beta | | | | | | | |
| | | subcomplex | | | | | | | |
| | | subunit 6 | | | | | | | |
| O75489 | NDUFS3 | NADH | 1.47E+07 | 5.60E+06 | 4.74E+06 | 9.62E+06 | 5.50E+06 | −1.23 | 0.298 |
| | | dehydrogenase | | | | | | | |
| | | [ubiquinone] iron- | | | | | | | |
| | | sulfur protein 3, | | | | | | | |
| | | mitochondrial | | | | | | | |
| Q9Y697 | NFS1 | Cysteine | 4.30E+06 | 1.23E+06 | 0.00E+00 | 4.21E+06 | 1.64E+06 | −1.06 | 0.468 |
| | | desulfurase, | | | | | | | |
| | | mitochondrial | | | | | | | |
| Q13423 | NNT | NAD(P) | 1.29E+07 | 2.53E+06 | 2.09E+06 | 2.91E+06 | 6.43E+06 | −1.04 | 0.480 |
| | | transhydrogenase, | | | | | | | |
| | | mitochondrial | | | | | | | |
| A8MXV4 | NUDT19 | Nucleoside | 1.81E+07 | 4.58E+06 | 7.57E+05 | 1.12E+07 | 7.55E+06 | −1.14 | 0.418 |
| | | diphosphate-linked | | | | | | | |
| | | moiety X motif 19, | | | | | | | |
| | | mitochondrial | | | | | | | |
| O60313 | OPA1 | Dynamin-like | 1.50E+07 | 3.49E+06 | 2.11E+06 | 9.75E+06 | 4.69E+06 | −1.13 | 0.387 |
| | | 120 kDa protein, | | | | | | | |
| | | mitochondrial; | | | | | | | |
| | | Dyna min-l | | | | | | | |
| | | ike 120 kDa | | | | | | | |
| | | protein, form S1 | | | | | | | |
| P07237 | P4HB | Protein disulfide- | 2.51E+07 | 6.39E+06 | 8.79E+06 | 1.00E+07 | 1.61E+07 | 1.24 | 0.375 |
| | | isomerase | | | | | | | |
| Q99497 | PARK7 | Protein deglycase | 9.21E+05 | 5.71E+05 | 0.00E+00 | 1.97E+06 | 0.00E+00 | −1.44 | 0.382 |
| | | DJ-1 | | | | | | | |
| P11177 | PDHB | Pyruvate | 5.34E+07 | 2.01E+07 | 2.98E+07 | 2.55E+07 | 1.41E+07 | −1.30 | 0.238 |
| | | dehydrogenase | | | | | | | |
| | | E1 component | | | | | | | |
| | | subunit beta, | | | | | | | |
| | | mitochondrial | | | | | | | |
| Q15118 | PDK1 | [Pyruvate | 9.61E+05 | 5.63E+05 | 0.00E+00 | 3.00E+06 | 6.33E+05 | 1.48 | 0.355 |
| | | dehydrogenase | | | | | | | |
| | | (acetyl-transferring)] | | | | | | | |
| | | kinase isozyme 1, | | | | | | | |
| | | mitochondrial | | | | | | | |
| Q15120 | PDK3 | [Pyruvate | 9.09E+05 | 4.36E+05 | 0.00E+00 | 0.00E+00 | 9.55E+05 | −1.17 | 0.451 |
| | | dehydrogenase | | | | | | | |
| | | (acetyl-transferring)] | | | | | | | |
| | | kinase isozyme 3, | | | | | | | |
| | | mitochondrial | | | | | | | |
| Q99623 | PHB2 | Prohibitin-2 | 4.36E+08 | 2.15E+07 | 1.22E+08 | 7.85E+07 | 6.77E+07 | −1.09 | 0.436 |
| Q9H2U2 | PPA2 | Inorganic | 8.10E+05 | 8.64E+05 | 7.64E+05 | 2.12E+06 | 9.59E+05 | 1.25 | 0.359 |
| | | pyrophosphatase 2, | | | | | | | |
| | | mitochondrial | | | | | | | |
| P30048 | PRDX3 | Thioredoxin- | 7.24E+07 | 2.48E+07 | 6.04E+07 | 8.36E+07 | 1.36E+07 | 1.23 | 0.336 |
| | | dependent | | | | | | | |
| | | peroxide | | | | | | | |
| | | reductase, | | | | | | | |
| | | mitochondrial | | | | | | | |
| Q96EY7 | PTCD3 | Pentatricopeptide | 1.06E+07 | 1.49E+06 | 0.00E+00 | 6.97E+06 | 2.52E+06 | −1.13 | 0.430 |
| | | repeat domain- | | | | | | | |
| | | containing protein 3, | | | | | | | |
| | | mitochondrial | | | | | | | |
| Q9Y606 | PUS1 | tRNA | 2.83E+06 | 1.25E+06 | 1.75E+06 | 4.21E+06 | 1.23E+06 | 1.26 | 0.301 |
| | | pseudouridine | | | | | | | |
| | | synthase A, | | | | | | | |
| | | mitochondrial | | | | | | | |
| Q8IXI1 | RHOT2 | Mitochondrial Rho | 7.10E+06 | 1.58E+06 | 0.00E+00 | 2.05E+06 | 2.81E+06 | −1.45 | 0.311 |
| | | GTPase 2 | | | | | | | |
| Q9H9B4 | SFXN1 | Sideroflexin-1 | 4.42E+07 | 8.56E+06 | 9.03E+05 | 1.63E+07 | 1.67E+07 | −1.29 | 0.360 |
| Q96NB2 | SFXN2 | Sideroflexin-2 | 7.68E+05 | 3.84E+05 | 0.00E+00 | 1.77E+06 | 4.53E+05 | 1.32 | 0.381 |
| Q9UJS0 | SLC25A13 | Calcium-binding | 1.99E+07 | 4.45E+06 | 2.32E+06 | 5.69E+06 | 8.30E+06 | −1.26 | 0.360 |
| | | mitochondrial carrier | | | | | | | |
| | | protein Aralar2 | | | | | | | |
| Q7KZF4 | SND1 | Staphylococcal | 4.16E+07 | 6.65E+06 | 5.39E+06 | 2.33E+07 | 7.37E+06 | −1.26 | 0.284 |
| | | nuclease domain- | | | | | | | |
| | | containing protein 1 | | | | | | | |
| Q9UJZ1 | STOML2 | Stomatin-like | 8.74E+07 | 1.99E+07 | 4.13E+07 | 3.23E+07 | 1.37E+07 | −1.27 | 0.194 |
| | | protein 2, | | | | | | | |
| | | mitochondrial | | | | | | | |
| Q9P2R7 | SUCLA2 | Succinyl-CoA | 2.51E+06 | 4.49E+05 | 1.36E+06 | 1.01E+06 | 2.36E+04 | −1.32 | 0.336 |
| | | ligase | | | | | | | |
| | | [ADP-forming] | | | | | | | |
| | | subunit beta, | | | | | | | |
| | | mitochondrial | | | | | | | |
| Q96I99 | SUCLG2 | Succinyl-CoA ligase | 6.19E+06 | 1.06E+06 | 0.00E+00 | 4.25E+06 | 7.86E+05 | −1.49 | 0.292 |
| | | [GDP-forming] | | | | | | | |
| | | subunit beta, | | | | | | | |
| | | mitochondrial | | | | | | | |
| O14925; | TIMM23; | Mitochondrial import | 2.03E+06 | 5.54E+05 | 0.00E+00 | 7.65E+05 | 1.63E+06 | 1.14 | 0.453 |
| Q5SRD1 | TIMM23B | inner membrane | | | | | | | |
| | | translocase subunit | | | | | | | |
| | | Tim23; Putative | | | | | | | |
| | | mitochondrial import | | | | | | | |
| | | inner membrane | | | | | | | |
| | | translocase subunit | | | | | | | |
| | | Tim23B | | | | | | | |
| Q3ZCQ8 | TIMM50 | Mitochondrial import | 3.88E+07 | 6.16E+06 | 1.24E+07 | 8.71E+06 | 6.61E+06 | −1.40 | 0.086 |
| | | inner membrane | | | | | | | |
| | | translocase subunit | | | | | | | |
| | | TIM50 | | | | | | | |
| Q9Y5J9 | TIMM8B | Mitochondrial import | 1.71E+07 | 9.53E+06 | 1.72E+07 | 9.77E+06 | 5.00E+06 | −1.21 | 0.370 |
| | | inner membrane | | | | | | | |
| | | translocase subunit | | | | | | | |
| | | Tim8 B | | | | | | | |
| Q9NS69 | TOMM22 | Mitochondrial import | 1.18E+07 | 6.60E+06 | 1.41E+07 | 1.33E+07 | 1.14E+06 | −1.05 | 0.472 |
| | | receptor subunit | | | | | | | |
| | | TOM22 homolog | | | | | | | |
| Q12931 | TRAP1 | Heat shock protein | 1.48E+08 | 5.55E+07 | 1.10E+08 | 1.61E+08 | 5.80E+07 | 1.27 | 0.209 |
| | | 75 kDa, | | | | | | | |
| | | mitochondrial | | | | | | | |
| P43897 | TSFM | Elongation factor Ts, | 1.80E+07 | 1.18E+07 | 8.08E+05 | 4.43E+07 | 6.46E+06 | −1.03 | 0.488 |
| | | mitochondrial | | | | | | | |
| Q16762 | TST | Thiosulfate | 1.43E+07 | 3.17E+06 | 1.16E+07 | 4.68E+06 | 1.89E+06 | 1.00 | 0.498 |
| | | sulfurtransferase | | | | | | | |
| A3KMH1 | VWA8 | von Willebrand | 1.57E+06 | 4.00E+05 | 0.00E+00 | 9.08E+05 | 7.06E+05 | −1.13 | 0.428 |
| | | factor A domain- | | | | | | | |
| | | containing protein 8 | | | | | | | |
| Q96TA2 | YME1L1 | ATP-dependent | 8.87E+06 | 3.17E+06 | 3.52E+05 | 9.35E+06 | 4.49E+06 | −1.01 | 0.495 |
| | | zinc | | | | | | | |
| | | metalloprotease | | | | | | | |
| | | YME1L1 | | | | | | | |

UniProt IDs: Uniprot accession IDs of identified mitochondrial proteins as defined by MitoCarta 2.0 (Calvo etal. 2016; PMID:26450961)
Protein names: Protein names of identified mitochondrial proteins
Protein description: Description of identified mitochondrial proteins
WT-1: Total peptide ion intensity determined by MaxQuant in replicate 1 of wild type (WT)
WT-2: Total peptide ion intensity determined by MaxQuant in replicate 2 of WT
CLPP KO-1: Total peptide ion intensity determined by MaxQuant in replicate 1 of CLPP knockout (KO)
CLPP KO-2: Total peptide ion intensity determined by MaxQuant in replicate 2 of CLPP KO
CLPP KO-3: Total peptide ion intensity determined by MaxQuant in replicate 3 of CLPP KO
Avg. fold change (CLPP KO/WT): Fold change of protein detected in CLPP knockout as compared to WT
p-Value significance: Proteins detected in CLPP KO versus WT using Students paired t test
Mitochondrial proteins identified with p-value 0.05 and |1.5| fold change are highlighted in bold.

Cellular Sensitivity to ADEP Correlates with Intracellular HsCIpP Expression Levels

Given that ADEP-induced cytotoxicity depends on HsCIpP, the effect of intracellular HsCIpP levels on the cell's sensitivity to ADEP was examined. HsCIpP overexpression was achieved using HEK293 T-REx overexpressing a C-terminally FLAG-tagged, full length HsCIpP (CLPP-FLAG) upon induction with doxycycline (Dox). Similarly, HEK293 T-REx+CLPP_{S153A}-FLAG overexpresses a C-terminally FLAG-tagged, full length inactive HsCIpP(S153A) mutant.

HsCIpP overexpression was confirmed by Western blot analysis. As shown in FIG. 4D, high expression of FLAG-tagged HsCIpP or FLAG-tagged HsCIpP_{S153A} was detected in HEK293 T-REx+CLPP-FLAG and HEK293 T-REx+CLPP_{S153A}-FLAG, respectively, in the presence of Dox (band I). Due to leakiness of the Dox-inducible promoter, expression of the FLAG-tagged HsCIpP proteins (WT or S153A mutant) was also detected in the absence of Dox, albeit at significantly lower levels (band I). Expression of endogenous HsCIpP was also observed (band III). Interestingly, an additional HsCIpP band (band II) that corresponds to a slightly larger HsCIpP protein but lacks the C-terminal FLAG-tag (α-FLAG panel), was observed in both HEK293 T-REx+CLPP-FLAG (with or without Dox) and in HEK293 T-REx+CLPP_{S153A}-FLAG (with Dox only) cells. Band II is likely the result of the C-terminal FLAG-tag being cleaved via HsCIpP's own proteolytic activity.

To determine the effect of intracellular HsCIpP expression on the cell's sensitivity to ADEP, HEK293 T-REx WT, CLPP^{-/-}, HEK293 T-REx+CLPP-FLAG and HEK293 T-REx+CLPP_{S153A}-FLAG cells were grown in the presence of ADEP-41 with or without Dox. Without Dox, both HEK293 T-REx+CLPP-FLAG and HEK293 T-REx+CLPP_{S153A}-FLAG were equally sensitive to ADEP-41 as WT, while CLPP^{-/-} was resistant (FIG. 4E, −Dox panel). Accordingly, the IC₅₀ for WT, HEK293 T-REx+CLPP-FLAG and HEK293 T-REx+CLPP_{S153A}-FLAG were similar within statistical errors (FIG. 4F). In the presence of Dox, the increase in intracellular HsCIpP level rendered HEK293 T-REx+CLPP-FLAG highly sensitive to ADEP-41 compared to WT (FIG. 4E, +Dox panel), resulting in a significant decrease in IC₅₀ (FIG. 4F). Importantly, HEK293 T-REx+CLPP_{S153A}-FLAG, which expresses native HsCIpP at the WT level but can only overexpress the proteolytically inactive HsCIpP mutant, maintains WT-like sensitivity to ADEP-41 (FIG. 4E, +Dox panel) and shows no change in IC₅₀ (FIG. 4F). These results clearly illustrate that the intracellular expression level of proteolytically active HsCIpP can modulate the cell's sensitivity to ADEP, with higher expression resulting in greater sensitivity.

Aside from HEK293 T-REx, the cellular sensitivity to ADEP-41 was investigated in several other commonly used cell lines. These include HeLa (regular), HeLa T-REx, U2OS and undifferentiated SH-SYSY (FIGS. 5B and C). The IC₅₀ for all cell lines were similar with undifferentiated SH-SYSY having a slightly higher value (FIG. 5C). However, U2OS and undifferentiated SH-SYSY have lower levels of HsCIpP than the other cell lines (about 20% and 40% relative to HEK293 T-REx, respectively) (FIG. 5D). Hence, in addition to the intracellular level of HsCIpP, ADEP sensitivity varies slightly by cell type.

### ADEP Activates the Intrinsic, Caspase-Dependent Apoptosis in HEK293 T-REx Cells

The cellular mechanism underlying ADEP's cytotoxicity on HEK293 T-REx cells was further investigated. First, morphological changes of HEK293 T-REx WT and CLPP^{-/-} exposed to ADEP-41 for 72 hours were examined by microscopy. ADEP-41-treated WT cells appeared compact and spherical, and exhibited a significant loss of adherence to the growth surface (FIGS. 6A and 7A). This is in stark contrast to DMSO-treated WT cells as well as to CLPP^{-/-} cells that are resistant to ADEP-induced cytotoxicity (FIGS. 6A and 7A). Furthermore, blebs were observed on the surface of affected WT cells (indicated with arrows in FIG. 6A).

Both cell shrinkage and surface blebs formation are strong indicators of apoptosis (Elmore, 2007). Apoptosis in HEK293 T-REx WT cells induced by ADEP-41 was confirmed by TUNEL assay indicating the fragmentation of chromosomal DNA that congregate into small, condensed bodies (appearing as multiple foci) upon compound treatment (FIG. 6A). In contrast, DMSO-treated WT cells have intact chromosomal DNA and were not TUNEL-labeled. Importantly, both ADEP-treated and DMSO-treated CLPP^{-/-} cells have normal nuclei and intact chromosomal DNA (FIG. 6A). Quantification of cells with positive TUNEL-labelling revealed that about 20 times more of the ADEP-41-treated WT cells were TUNEL-positive compared to DMSO-treated ones, or compared to CLPP^{-/-} cells treated with ADEP-41 or DMSO (FIG. 6B).

Subsequently, the intracellular expression of signature apoptotic protein markers was examined by Western blotting. The expression of both HsCIpP and HsCIpX was also examined. Treatment with ADEP for 24 or 72 hours did not change HsCIpP levels in WT cells compared to DMSO (FIG. 6C). In contrast, HsCIpX was no longer detected in WT cells treated with ADEP-41 for either duration (FIG. 6C), despite the fact that HsCIpX is not degraded by the ADEP-activated HsCIpP in vitro (FIGS. 3E-G). To determine whether the loss of HsCIpX has any potential role in ADEP-induced apoptosis, a CLPX gene disruption was obtained using CRISPR-Cas9, which reduces HsCIpX expression by ≥97% (ΔCLPX*; FIG. 7B). Significant reduction of HsCIpX levels had no statistically significant impact on the cells' sensitivity to ADEP-28 regardless of HsCIpP expression (FIG. 7C). Hence, without wishing to be bound by theory, the loss of HsCIpX in ADEP-treated HEK293 T-REx WT cells may be part of a stress response mechanism. Alternatively, HsCIpX could be degraded by other mitochondrial proteases.

After 24 hours of ADEP-41 treatment, no activation-specific cleavage was observed for Caspase-8, Caspase-9 or Caspase-3. However, the anti-apoptotic Mcl-1 protein was degraded in WT cells, but not in CLPP^{-/-} cells (FIG. 6C). Conversely, the pro-apoptotic PUMA protein was significantly upregulated in WT cells in contrast to CLPP^{-/-} cells (FIG. 6C). These changes in the intracellular levels of Mcl-1 and PUMA are strong indications for the initiation of the intrinsic apoptotic pathway.

After 72 hours of ADEP-41 treatment, the degradation of Mcl-1 in WT cells remained clearly observable (FIG. 6C). Notably, there was no change in the level of anti-apoptotic Bcl-2 protein, suggesting that the loss of Mcl-1 is a specific cellular response to the ADEP-41 treatment. Importantly, activation-specific cleavage was detected for both Caspase-9 and Caspase-3, which produced the characteristic p37 and p35 fragments for Caspase-9, and p19/p17 for Caspase-3 (FIG. 6C). Furthermore, no activation-specific cleavage was observed for Caspase-8 (FIG. 7D). Given that Caspase-9 is functionally associated with the intrinsic apoptotic pathway while Caspase-8 is linked to the extrinsic pathway (Mcllwain et al., 2013), it is concluded that treatment of HEK293 T-REx WT cells with ADEP activates the intrinsic apoptotic pathway, leading to cell death.

### ADEP Induces Mitochondrial Fragmentation and Abolishes Oxidative Phosphorylation in HEK293 T-REx Cells

Given that mitochondrial fragmentation and outer membrane permeabilization (MOMP) are hallmarks of apoptosis (Landes and Martinou, 2011), the impact of ADEP on mitochondrial morphology and oxidative phosphorylation (OXPHOS) was further investigated. Mitochondria in HEK293 T-REx WT and CLPP^{-/-} treated with ADEP-41 or DMSO for 24 and 72 hours were examined below the diffraction limit by super resolution 3D Structured Illumination Microscopy (3DSIM). As shown in FIG. 6D, HEK293 T-RExWT cells treated with ADEP-41 already showed clear signs of mitochondrial fragmentation after 24 hours compared to DMSO-treated cells. Furthermore, the fragmentation observed was very significant after 72 hours of treatment. In contrast, CLPP^{-/-} cells treated with ADEP-41 showed no change in mitochondrial morphology for either duration.

MOMP is known to dissipate the mitochondrial electrochemical gradient (Δψ) during apoptosis (Kroemer and Reed, 2000), which abolishes OXPHOS. As such, OXPHOS in WT and CLPP^{-/-} cells treated with ADEP-28 or DMSO for 24 hours was examined using the Seahorse extracellular flux (XF) analyzer. Notably, OXPHOS in ADEP-28-treated WT cells was largely abolished, as exemplified by the large reduction in oxygen consumption rate (OCR) associated with basal respiration (˜86%) and ATP synthase activity (˜91%), relative to DMSO-treated WT cells (FIG. 7E and F upper panel).

Furthermore, ADEP-treated WT cells showed a higher basal extracellular acidification rate (ECAR) that was largely unresponsive to ATP synthase inhibition (FIG. 7F lower panel), showing the cell's reliance on glycolysis for energy metabolism as a result of losing OXPHOS. Without wishing to be bound by theory, both events can be explained with Δψ dissipation as a result of MOMP.

The fragmentation of mitochondria and loss of OXPHOS from Δψ dissipation both provide confirmative evidence that ADEP induces apoptosis in HsCIpP-expressing cells.

### ADEP-Binding Induces Conformational Changes in the HsCIpP Tetradecamer

To further understand the mechanism by which ADEP binding leads to HsCIpP dysregulation, the structure of HsCIpP co-crystallized with ADEP-28 was determined (FIG. 8A) at 2.8 Å resolution (Table 2). ADEP-28 has high binding affinity for HsCIpP (FIG. 2E). In the crystal, the asymmetric unit contains a single heptameric ring that forms the HsCIpP tetradecamer with a second ring related to the first by crystallographic symmetry (FIG. 8A-E and Table 2). Of the 277 residues in HsCIpP, no electron density was observed for residues 252-277 (chains A-D) or 251-277 (chains E-G) at the C-terminus, whereas residues 1-57 that contain the mitochondrial transport signal were omitted during cloning (FIG. 8B). It was also observed that extra electron density attached to the thiol group of Cys86 in each subunit, indicating covalent modification (FIG. 8F). Mass spectrometry analysis identified this modification as potentially being persulfidation of the cysteine thiol.

As observed for other CIpPs, ADEP-28 bound to the hydrophobic pocket formed by two neighbouring HsCIpP subunits (FIG. 8A). Clear electron densities for all seven ADEP-28 molecules per heptameric ring were observed (FIG. 8C). The N-terminal axial loops were ordered (FIGS. 8A, D, and E), as observed previously in the ADEP-bound EcCIpP (Li et al., 2010), N. meningitidis CIpP (NmCIpP) (Goodreid et al., 2016), and M. tuberculosis CIpP (MtCIpP) (Schmitz et al., 2014). ADEP-28 binding enlarged the HsCIpP's axial pore (FIG. 8A). Interestingly, ADEP-28-HsCIpP adopts a compact conformation (FIG. 8A), which has not been seen in other ADEP-bound CIpP structures characterized to date (Goodreid et al., 2016; Lee et al., 2010; Li et al., 2010; Schmitz et al., 2014); and PDB 5VZ2. The inventors will first describe the ADEP binding pocket and then discuss the distal molecular rearrangements observed.

The binding of ADEP-28 to HsCIpP induces local structural changes at and around the binding pocket between two neighbouring subunits, forming a highly complementary surface for the ADEP molecule (FIGS. 9A and 10). Normally, the N-terminal β-1-β0 hairpins forming the axial loops are disordered in the absence of ADEP (FIG. 8A). An ionic interaction between R78 of one subunit and E109 of the neighbouring subunit secures the interface between the subunits (FIG. 9Ai). Upon ADEP-28 binding, ordered axial loop formation places E64 between R78 of the same subunit and E109 of the neighbouring subunit, creating three new hydrogen bonds (H bonds) that also involves R78 interacting with the intrasubunit E82 (FIG. 9Aii). These interactions stabilize a widened surface with the bound ADEP-28 acting as a wedge.

At the ADEP-28/CIpP interface, two Tyr residues, Y118 of one subunit and Y138 of the neighbouring subunit, form H-bonds with carbonyl carbon atoms of the depsipeptide ring (FIGS. 9Aii and 10). Similar interactions have been reported for other bacterial CIpP-ADEP complexes (Goodreid et al., 2016; Li et al., 2010; Schmitz et al., 2014). In addition, a H₂O-mediated H-bond is observed between Q107 and the amide carbonyl connecting the hydrophobic tail and 3,5-difluorophenyl moieties of ADEP-28. A Trp residue (W146) in

HsCIpP forms a hydrophobic stacking interaction with the 3,5-difluorophenyl moiety of ADEP-28 (FIGS. 9Aii and 10A). The corresponding residues in bacterial CIpPs all have smaller aliphatic side chains, which yield a smaller hydrophobic surface for the difluorophenyl group (FIG. 10B).

The depsipeptide ring is solvent-exposed but with its nitrogenous, heterocyclic rings forming stacking interactions with the aromatic or hydrophobic residues in the ADEP-binding pocket. In particular, the methyl piperidine ring of ADEP-28 (FIG. 6Aii on far right of ADEP-28 when viewed as shown) sits directly on top of H168 of β5. The relatively small side chain of the His residue is likely to provide additional space to accommodate the methyl piperidine ring and in turn strengthens the ADEP-28-HsCIpP interaction, resulting in the high apparent binding affinity observed (FIG. 2E). In contrast, the equivalent residue in bacterial CIpPs is either a Tyr, Phe or Met, all of which are bulkier than the His residue of HsCIpP (FIG. 10B).

As mentioned above, binding of ADEP-28 induces the formation of the N-terminal β-1-β0 hairpins (residues 58-74), creating ordered axial loops (FIGS. 8A, D and E). While the β-1 and β0 strands are highly structured in all subunits, segments of the β-hairpin loops formed by residues 65-69 in subunits C to G have ambiguous electron density insufficient to confidently assign side chain positions. These residues have significantly higher B-factors than those of the rest of the HsCIpP molecule (FIG. 8E), indicating greater flexibility. Each axial β-hairpin is anchored to the head domain of the same subunit through the interactions of E64 on β-1 strand and Y73 on β0 strand with R78 and R81 on aA helix, respectively (FIG. 9Bi, ii). The continuous stretch of 7 hydrophobic residues (L58-V63) comprising the short N-terminal loop and the β-1 strand form hydrophobic interactions with an extensive surface formed by Y73 (β0), Y76 (αA), L80 (αA), V95 (αB), L98 (αB), F105 (αB), L106 (αB) of a neighbouring subunit, and I75 (αA) and Y76 (αA) of the same subunit (FIG. 9Biii). These interactions result in the retraction of the N-terminus away from the 7-fold axis, leading to HsCIpP's axial pore widening. This has also been observed to occur in ADEP-bound EcCIpP (Li et al., 2010).

The compaction of the ADEP-28-HsCIpP complex was unexpected and is a direct consequence of molecular rearrangements at and around the handle region comprised mainly of β7 and αE resulting in significant reshuffling of the residues at the interface of the two apposing HsCIpP rings (FIG. 9Ci,ii). At the subunit level, residues 182-187 (182-191 in chain B) that are part of β7-strand of the handle region in apo HsCIpP show no clear electron density upon ADEP-28 binding, suggesting local structural disorder of the handle region (FIG. 9Cii). Furthermore, the ensuing αE-helix is shortened by the unwinding of two helical turns to an ordered loop that extends into HsCIpP's lumen due to a kink that starts at residue Q194 (FIG. 9Cii).

In apo HsCIpP, the interface of two heptameric rings consists of an intricate network of H-bonding and ionic interactions. The highly conserved oligomerization sensor R226 (αF-β8 loop) participates in intra-ring interactions with Q187 (β7-αE loop) and D190 (αE) of a neighbouring subunit and in inter-ring interactions with E225 (α′F) of the apposing subunit (FIG. 9Ci). Residues Q179 (β6-β7 loop) and K202 (αE) also form H-bonds with T189 (α′E) of the apposing subunit. Residue Q194 (αE) forms a H-bond with the catalytic D227 residue of a neighbouring subunit to further cement intra-ring subunit contacts (FIG. 9Ci). These interactions are conserved and highly symmetrical across the equatorial region of the HsCIpP tetradecamer and are likely essential for a properly aligned catalytic triad (S153, H178, D227).

In ADEP-28-bound HsCIpP, significant rearrangements of the intra- and inter-ring contacts occur as the long αF-β8 loops of apposing subunits come closer together to form part of the new interface. Remarkably, the catalytic triads become distorted as a result, such that H178 of one subunit now interacts with D227 of the apposing subunit and vice versa, while the original intra-subunit interaction between H178 and S153 and the inter-subunit interaction between D227 of one subunit with Q194 of the adjacent subunit are both lost (FIG. 9Cii). His178 forms a bond with Q194 of an apposing subunit, while D227 forms a bond with S181 of another apposing subunit. Furthermore, new intra-ring and inter-ring contacts are borne out of this structural rearrangement. These include the R174 (β6)-E197 (α′E) ionic interaction, the interactions of residues K202 and Y206 in α′E with Q194 and E196 of the apposing subunit, and the bond between S181 (β6-β7 loop) and the catalytic D227 of the apposing ring. Interestingly, despite these major changes at the interface, the inter-ring interaction of R226 in one subunit with E225 of the apposing subunit is preserved, albeit with a shift in the relative positions of the residues (FIG. 9Cii). The overall result of these molecular rearrangements is the compaction of the HsCIpP tetradecamer.

### Compaction of HsCIpP Results in the Formation of Equatorial Side Pores

This structure of ADEP-bound HsCIpP highlights a novel role of the catalytic triad residues in stabilizing the compact conformation. Several distinct, compact conformations have been observed in the crystal structures of CIpP from E. coli (Kimber et al., 2010), L. monocytogenes (Zeiler et al., 2013), M. tuberculosis (Ingvarsson et al., 2007), P. falciparum (El Bakkouri et al., 2010), S. aureus (Ye et al., 2013), and S. pneumoniae (Gribun et al., 2005) (FIG. 11). In addition, SaCIpP was also crystallized in a compressed conformation (Zhang et al., 2011). Here the compact conformation is defined as a shortened CIpP cylinder losing one or more turns at the N-terminus of the αE helix and in many cases concomitant with the loss of density for the β7 strand. The compressed conformation refers to an even shorter CIpP cylinder caused by a kink in the αE helix resulting in the formation of two smaller helices.

These CIpP structures (FIG. 11) have varying degrees of compaction with the distorted catalytic triads engaging in distinct interactions with nearby residues. Among these, the LmCIpP1 and SaCIpP compact structures show engagement of one or more of the catalytic triad residues in inter-ring interactions (FIG. 11) as does the compact ADEP-28-HsCIpP structure (FIG. 9ii), albeit having different interacting residues. The compressed SaCIpP shows inter-ring interactions of the catalytic triad but they are mediated by a sulfate group (FIG. 11).

The compact structure of ADEP-28-HsCIpP exhibits small, equatorial side pores that presumably enlarge to facilitate the egress of cleaved peptides from the CIpP barrel (FIG. 12A). These are in close proximity to the peptide substrate binding site and are formed by residues in the vicinity of the hinge region. Other compact CIpP structures exhibit equatorial side pores of varying sizes and shapes depending on the degree of compaction (FIG. 12B). Interestingly, in the compact ADEP-28-HsCIpP, the unraveling of two N-terminal helical turns of the αE helix results in a structured loop (spanning residues 188-194), a segment of which occupies the location of the bound peptide substrate near the catalytic site and the side pore (FIG. 12A). A similar phenomenon is observed in the compact structure of PfCIpP, as well as in the compressed structure of SaCIpP, where the αE helix is kinked in such a way that the ensuing residues of a short loop occupy the peptide binding site. Therefore, the compact and compressed structures of CIpP are likely intermediates that are populated during CIpP's functional cycle in which a cleaved peptide is poised for release from the CIpP lumen through the equatorial side pores.

Analysis of the available CIpP structures from different species where both extended and compact conformations have been solved shows that the 7 catalytic Ser residues of a CIpP heptameric ring form a plane that is parallel to that formed by the other 7 catalytic Ser residues in the apposing heptamer (rotation angle, θ=−3° to +1.5°) (FIG. 13). The distance between each plane's center of mass (with each plane defined by the coordinates of the Ser Cα atoms) is ˜30 Å, and this ‘height’ decreases by as much as 8 Å upon compaction. Compaction is accompanied by rotation of the catalytic Ser planes relative to each other by as much as 23° and a concomitant, general increase in the distances between the 7 coplanar Ser residues of a ring (FIG. 13). The same is true for the other catalytic residues. For SaCIpP where a third, compressed structure is available, the ‘height’ is further reduced to ˜20 Å, while the rotation angle between catalytic Ser planes is reduced to just 6.4°, presumably to relieve the strain caused by compression (FIG. 13). Thus, collectively, these CIpP structures may constitute distinct structural intermediates in the conformational landscape of CIpP, whether as a result of activation by ADEP or CIpX or by natural breathing motions.

### Solution Structure of ADEP-Bound HsCIpP Characterized by Small Angle X-Ray Scattering (SAXS)

The global conformational change in the HsCIpP structure upon ADEP binding was further examined in solution using SAXS (FIG. 14A). HsCIpP, HsCIpP+DMSO (control for DMSO's effect) and HsCIpP+ADEP-28 showed molecular mass (MM) values that correspond to tetradecamers (Table 5). The small increase in MM for HsCIpP+ADEP-28 suggested that ADEP-28 molecules were bound to HsCIpP. Pair distance distribution functions, p(r), were then generated (FIG. 14B) and the values for the radius of gyration (Rg) and maximum dimension (Dₘₐₓ) were determined. HsCIpP and HsCIpP+DMSO showed similar p(r) profiles (FIG. 14B), and also similar Rg and Dₘₐₓ values (Table 5). All values were in agreement with the calculated size and dimensions of HsCIpP's crystallographic structure. Importantly, the addition of ADEP-28 changed HsCIpP's p(r) profile, shifting the maximum p(r) and Dₘₐₓ towards higher values (FIG. 14B).

TABLE 5
Size and dimension properties determined for HsCIpP by SAXS

| | SAXS | | | Crystallography⁶ | |
|---|---|---|---|---|---|
| | | | | | HsCIpP |
| | | HsCIpP + | HsCIpP + | | + |
| Properties | HsCIpP | DMSO | ADEP-28 | HsCIpP | ADEP-28 |
| Rg (Å)¹ | 44.5 | 44.3 | 45.9 | 42.9 | 42.7 |
| Dₘₐₓ (A)¹ | 130 | 132 | 140 | 136 | 130 |
| MM_{Experimental} (kDa)² | 337 ± 21 | 344 ± 11 | 376 ± 21 | | |
| MM_{Theoretical} (kDa)³ | 340 | 340 | 351⁴ | | |
| Oligomeric state⁵ | 13.9 | 14.2 | 15.1 | 14-mer | 14-mer |

¹Properties experimentally determined using final merged SAXS curves. Rg is radius of gyration. Dₘₐₓ is maximum dimension of the molecule.
²MMExperimental is the average molecular mass determined from eleven SAXS curves.
³Calculated from the amino acid sequence using the Protparam program (http://web.expasy.org/protparam/).
⁴The MM_{Theoretical} of compound-bound CIpP was calculated taking into account additional 14 molecules of ADEP-28 (MMTheoretical = 784.89 g/mol).
⁵For the SAXS data, the oligomeric state is obtained by dividing MM_{Experimental} by the MM_{Theoretical} of the monomer (24.2 kDa). For the crystallographic data, the oligomeric state refers to the established biological complex.
⁶Values calculated from the crystallographic structures by the program Hydropro (http://leonardo.inf.um.es/macromol/programs/hydropro/hydropro.htm).

Using SAXS data, low resolution dummy atoms models (DAMs) were generated for HsCIpP+DMSO and HsCIpP+ADEP-28 (FIG. 14C). Notably, in the presence of DMSO, HsCIpP adopts a cylindrical shape in solution that is compatible with the published structure of apo HsCIpP (Kang et al., 2004). The ADEP-bound HsCIpP DAM showed an increased radial and axial occupancy with dummy atoms models, adopting an ellipsoidal shape. The superposition of our ADEP-bound HsCIpP crystallographic structure on this DAM (FIG. 14C) showed a good fit at radial positions, supporting the existence of this compact conformation. Nevertheless, the empty spaces observed at the top and bottom regions of the ADEP-bound HsCIpP DAM, and the differences between properties calculated from the crystal structure and determined from the SAXS data (Table 5) indicate the likely presence of other HsCIpP conformers in solution.

### Discussion

The present disclosure describes the first detailed biochemical and biophysical characterization of the molecular interactions between ADEP and mitochondrial HsCIpP, as well as the first report on the physiological impact of these interactions on human cancer cells. Lowth et al., 2012 had suggested earlier that some ADEP analogs that they tested may have affected HsCIpP, although they did not characterize those interactions further and provided no insight or evaluation on the use of ADEP in cancer treatment.

The present identification of ADEPs that activate HsCIpP was a fortuitous finding stemming from inventors' work on developing antibiotics targeting the bacterial CIpP.

The co-crystal structure of the ADEP-28 and HsCIpP recapitulates previous findings in ADEP-bound bacterial CIpPs, while providing new insights into the protease's functional cycle. Notably, the compact conformation of the ADEP-28-HsCIpP complex may constitute a putative intermediate state. While the degradation of substrates requires a properly aligned Ser-His-Asp catalytic triad that is found only in the extended conformation of the tetradecameric CIpP, additional conformations are also required to facilitate the other steps in the protein degradation process, such as the release of peptide fragments from inside the CIpP lumen. Without wishing to be bound by theory, biochemical, and biophysical experiments have thus pointed towards a dynamic CIpP tetradecamer that can extend, compact, and compress, with the flexible handle region acting as a hinge point for achieving these various conformations (Kimber et al., 2010; Liu et al., 2014). The structure of ADEP-28-HsCIpP highlights the essential role of the handle region to achieve a compact conformation whilst bound by ADEP-28 at the activator site, providing the first structural evidence that an ADEP-activated CIpP is not locked in the extended conformation, contrary to the proposal by Gersch and co-workers (Gersch et al., 2015). Instead, the ADEP-bound CIpP is sufficiently flexible and can dynamically assume conformations found in other stages of the CIpP degradation cycle. Remarkably, the X-ray structure also unveils a previously unknown structural role for the catalytic triad outside of proteolysis that appears essential for stabilizing the compact conformation.

Importantly, the interaction of ADEP with HsCIpP produces a cytotoxic effect on cells that manifests via the intrinsic, caspase-dependent apoptosis in cancer cells. An important utility from these results is that ADEPs are useful as novel therapeutic compounds for cancer treatment. Modulation of the cell's sensitivity to ADEPs by intracellular HsCIpP expression enables the fine-tuning of the ADEP chemical structure, such that new analogs can be developed with better abilities to distinguish and target cancer cells that express high levels of HsCIpP without affecting normal, healthy cells. The biophysical and structural data on the interaction between ADEP and HsCIpP are useful in identifying key chemical features in the ADEP structure that define its potency and will facilitate the design of better analogs.

The mitochondrion is a vital organelle in the human cell that has many essential biological functions. These include energy metabolism, signaling, and apoptosis. Consequently, a dysfunctional mitochondrion may give rise to a wide range of diseases including cancer. In this disclosure, the identification of compounds, i.e. ADEP analogs, that dysregulate the activity of a critical protease present in the mitochondrial matrix termed CIpP, i.e. HsCIpP, is described. These ADEP analogs dysregulate the protease activity of CIpP causing it to degrade proteins in the mitochondrion in a dysregulated manner, resulting in apoptotic cell death. The co-crystal structure of CIpP with an ADEP analog was obtained which revealed a novel conformation for the protease. The disclosure shows that ADEP analogs kill cancer cells which express high level of HsCIpP. Hence, ADEP analogs are useful for cancer treatment in targeting cancer cells expressing high levels of HsCIpP.

### Example 2: Additional Experiments on ADEP Analogs Materials and Methods

### Cell Line Maintenance and Propagation

HEK293 T-REx wild-type (WT) and HEK293 T-REx CLPP^{-/-} cells were obtained from Professor Aleksandra Trifunovic (University of Cologne, Germany). MDA-MB-231 and MDA-MB-468 were obtained from Professor Lilianna Attisano (University of Toronto, Canada). MCF-7 cells were obtained from the lab of Professor Grant Brown (University of Toronto, Canada). MDA-MB-231 is an invasive ductal carcinoma cell line. It is triple-negative, i.e. MDA-MB-231 cells do not express estrogen receptors, progesterone receptors, and have no ERBB2 amplification. These cells have metastatic origin and was isolated from pleural effusion of a breast cancer patient. MCF-7 is another invasive ductal carcinoma cell line originated from pleural effusion. MCF-7 cells express estrogen receptors and do not express progesterone receptors and do not have ERBB2 amplification. MDA-MB-468 cells were extracted from a pleural effusion of mammary gland and breast tissues, and are useful for the study of metastasis, migration, and breast cancer proliferation.

Unless otherwise stated, both HEK293 T-REx WT and CLPP^{-/-} were propagated and maintained with Dulbecco's Modified Eagle Media (DMEM) (Gibco) supplemented with 10% fetal bovine serum (FBS) (Gibco), 100 U/mL penicillin-streptomycin (Gibco) and 2 mM L-glutamine (Gibco), while MDA-MB-231, MDA-MB-468 and MCF-7 cells were grown in DMEM:F12 (Gibco) with the same supplements. All cultures were kept at 37° C. under a moist atmosphere with 6% CO₂. All cells were passaged at least 3 times prior to use.

### Genetic Knockdown of CLPP in MDA-MB-231 by CRISPR-Cas9

The genetic knockdown of CLPP in MDA-MB-231 was achieved via the CRISPR-Cas9 protocol as outlined in (Cong et al., 2013). Briefly, the single-strand DNA oligos CLPP Exon1 pX330 F (5′-caccggcgtgcggagggatgtggcc-3′) (SEQ ID NO:4) and CLPP Exon1 pX330 R (5′-aaacggccacatccctccgcacgcc-3′) (SEQ ID NO:5) were phosphorylated at their 5′-ends with T4 PNK (New England Biolabs) and annealed together by gradual cooling from 95° C. to 25° C. at a rate of 5° C./min in a thermocycler. The phorphorylated annealed oligo duplex was then ligated to a pX330 plasmid already digested with FastDigest BbsI (New England Biolabs) and dephosphorylated with FastAP (Thermo Scientific), using the T4 DNA ligase (New England Biolabs). The ligation reaction was then used to transform chemically competent Escherichia coli DH5a cells, followed by selection of ampicilin-resistant colonies on LB-agar plates supplemented with 100 μg/mL of the antibiotic. The resultant pX330-ΔCLPP plasmid was isolated using the PureLink Quick Plasmid Miniprep Kit (Invitrogen) and sequence-verified.

To generate MDA-MB-231 CLPP-KD, WT cells were first passaged in DMEM:F12 media without any antibiotics. These cells were then grown in 6-well tissue culture plates to near confluence in the absence of antibiotics. A DNA transfection mixture containing 1.7 μg of pX330-ΔCLPP and 100 nmol of the single-strand DNA oligo, CLPP Exon1 ssODN (5′-gtagttccgccatcggacggaagccgaccggggcgtgcggagggtgataataatgaatattggtagggg gggcccgggtggcgtcatgc-3′) (SEQ ID NO:6) was prepared using the JetPrime in vitro transfection reagent (Polyplus) following the manufacturer's protocol. Cells were incubated in the presence of the transfection mixture for 5 hours inside an incubator, after which a media change using fresh DMEM:F1 without antibiotics was performed and cells were maintained in the incubator for an additional 16-18 hours prior to passaging of cells to a larger tissue culture dish. At this time, some cells were kept for Western blotting to monitor the expression of CLPP. The transfection procedure was repeated to maximize the inhibition of CLPP expression.

Clonal populations of MDA-MB-231 CLPP-KD were generated by diluting the transfected cells and plating them in 96-well tissue culture plate at approximately 1 cell per 200 μL of media per well. Successful isolation of single cells was determined by visual examination of the wells, and their expansion into clonal populations was carried out for 1-2 weeks. Individual clonal populations were then transferred into larger tissue culture dishes for continued expansion. The successful knock down (KD) of CLPP was determined by Western blotting.

### Western Blotting

The same procedure as detailed under Example 1 for Western blotting was used on MDA-MB-231 WT and CLPP-KD cells. GAPDH is blotted to provide a sample-loading control.

### Cytotoxicity Test

Assessment of cytotoxicity of ADEP-14 on human breast cancer cell lines (MDA-MB-231 WT, MDA-MB-231 CLPP-KD, MDA-MB-468, and MCF-7) and HEK cells (HEK293 T-Rex and HEK293 T-REx CLPP) were determined using the methods as described above in Example 1. Briefly, cells were grown for at least 24 hours to allow proper adherence to the growth surface, and then ADEP-14 were serially diluted and introduced to the tissue cultures via fresh growth media. A total of four independent replicates were prepared for each cell line and for each growth condition used. Cells were grown in the presence of ADEP/DMSO for 72 hours. Cytotoxicity test with mitomycin-c, an antitumour antibiotic that inhibits DNA synthesis, was also conducted for comparison. Sulforhodamine B (SRB) staining method was used to quantify cell survival.

### Scratch Wound Healing Assay

MDA-MB-231 WT and CLPP-KD cells were grown to near confluence in 6-well tissue culture plates. Prior to scratch wound generation, all cultures were serum-starved by growing in serum-free DMEM:F12 media for 24 hours to inhibit new rounds of cell division, so to ensure that all wound closure events must originate from cell migration alone. Scratch wounds were generated by manually scratching the cell monolayer using a sterile pipette tip in a single, unidirectional stroke while keeping the tilt of the pipette tip constant.

A sterile ruler was used to guide the scratching action to ensure consistency across cultures. The positions of the wounds were marked with a permanent marker on the bottom side of each culture as positional reference for repeated imaging of the same wound areas. Cell debris was removed by gently washing the cultures with fresh serum-free DMEM:F12. The cells were then incubated in fresh serum-free DMEM:F12 containing ADEP-14 at 0, 100 nM, 200 nM, 300 nM, 400 nM, and 500 nM over 48 hours, during which the wounds were imaged using a Nikon TMS inverted microscope equipped with an OMAX A3550S digital camera and the ToupView software (ver. X64, 3.7.13865.20190127; ToupTek). Image analysis and scratch wound quantification were performed using the Bowhead software package (Engel et al., 2018), by measuring the area of scratch wounds (their perimeters outlined) at 0-hr and 48-hr time points for both WT and CLPP-KD, with or without ADEP-14. Normalization of data was performed using the 0-hr scratch wound area as reference. The error bars shown refer to the standard deviation across three independently constructed replicates (see FIG. 16B).

### Results and Discussion

ADEP-14 is Less Toxic to Cells with Lower Level of CIpP

The dependency of ADEP-induced cytotoxicity on HsCIpP levels was further determined in breast cancer cell lines. HsCIpP knock down in MDA-MB-231 CLPP-KD cells were confirmed by Western blot analysis (FIG. 15A). MDA-MB-231 CLPP-KD showed 10× lower response to ADEP-14 induced cytotoxicity comparing to MDA-MB-231 WT (FIG. 15B). ADEP-14 was also shown to be cytotoxic to breast cancer cell lines MDA-MB-468 and MCF-7 (Table 6). A summary of ADEP-induced cytotoxicity in various cell lines, including breast cancer cell lines, is shown in Table 6. The cytotoxic effects of ADEP-14 on breast cancer cell lines are comparable to antitumour agent mitomycin-C.

TABLE 6
ADEP-induced cytotoxicity in various cell lines

| | IC₅₀ for | IC₅₀ for |
|---|---|---|
| | ADEP-14 | Mitomycin-C |
| Cell Line | (μM) | (μM) |
| HEK293 T-REx | 1.01 ± 0.05 | n/d |
| HEK293 T-REx CLPP^{-/-} | >10* | n/d |
| MDA-MB-231 | 1.35 ± 0.O4 | 0.8 ± 0.2 |
| MDA-MB-231 CLPP-KD | >10* | n/d |
| MDA-MB-468 | 0.95 ± 0.06 | 1.5 ± 0.4 |
| MCF-7 | 1.6 ± 0.2 | 0.4 ± 0.2 |

*IC₅₀ estimated by visual examination, due to lack of minimum plateau in cell viability data that prohibits meaningful data analysis using the Hill model.
KD = knock down
n/d = not determined

### ADEP-14 Impairs MDA-MB-231 Cell Migration in 2D Culture

The effects of ADEP on cell migration was determined in a scratch wound healing assay in MDA-MB-231 WT and CLPP-KD cells. As shown in FIG. 16A, the presence of ADEP-14 impaired 2D migration of MDA-MB-231 WT cells. MDA-MB-231 CLPP-KD cells appeared to be less efficient in 2D migration. The assay also showed that migration of MDA-MB-231 CLPP-KD cells were unaffected by the presence of ADEP-14. Normalized quantification of wound closure is shown in FIG. 16B.

Cell migration is involved in cancer metastases. The inhibition of cell migration in MDA-MB-231 WT cells by ADEP-14 show that ADEP analogs are useful for inhibiting, treating or preventing cancer metastasis, including breast cancer metastasis.

While the present disclosure has been described with reference to what are presently considered to be the preferred example, it is to be understood that the disclosure is not limited to the disclosed example. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta crystallographica Section D, Biological crystallography 66, 213-221.

Alexopoulos, J.A ., Guarne, A., and Ortega, J. (2012). CIpP: a structurally dynamic protease regulated by AAA+ proteins. J Struct Biol 179, 202-210.

Amor, A. J., Schmitz, K. R., Sello, J. K., Baker, T. A., and Sauer, R. T. (2016). Highly Dynamic Interactions Maintain Kinetic Stability of the CIpXP Protease During the ATP-Fueled Mechanical Cycle. ACS Chem Biol 11, 1552-1560.

Barbosa, L. R. S., Spinozzi, F., Mariani, P., and Itri, R. (2013). Small-Angle X-Ray Scattering Applied to Proteins in Solution. In Proteins in Solution and at Interfaces: Methods and Applications in Biotechnology and Materials Science (Wiley), pp. 49-72.

Bewley, M. C., Graziano, V., Griffin, K., and Flanagan, J. M. (2006). The asymmetry in the mature amino-terminus of CIpP facilitates a local symmetry match in CIpAP and CIpXP complexes. Journal of Structural Biology 153, 113-128.

Bram, E., Ifergan, I., Shafran, A., Berman, B., Jansen, G., and Assaraf, Y. G. (2006). Mutant Gly482 and Thr482 ABCG2 mediate high-level resistance to lipophilic antifolates. Cancer Chemother Pharmacol 58, 826-834.

Brotz-Oesterhelt, H., Beyer, D., Kroll, H. P., Endermann, R., Ladel, C., Schroeder, W., Hinzen, B., Raddatz, S., Paulsen, H., Henninger, K., et al. (2005). Dysregulation of bacterial proteolytic machinery by a new class of antibiotics. Nat Med 11, 1082-1087.

Calvo, S. E., Clauser, K. R., and Mootha, V. K. (2016). MitoCarta2.0: an updated inventory of mammalian mitochondrial proteins. Nucleic acids research 44, D1251-1257.

Carney, D. W., Schmitz, K. R., Truong, J. V., Sauer, R. T., and Sello, J. K. (2014). Restriction of the conformational dynamics of the cyclic acyldepsipeptide antibiotics improves their antibacterial activity. J Am Chem Soc 136, 1922-1929.

Cole, A., Wang, Z., Coyaud, E., Voisin, V., Gronda, M., Jitkova, Y., Mattson, R., Hurren, R., Babovic, S., Maclean, N., et al. (2015). Inhibition of the Mitochondrial Protease CIpP as a Therapeutic Strategy for Human Acute Myeloid Leukemia. Cancer Cell 27, 864-876.

Cong, L., Ran, F.A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.

Csizmadia, V., Hales, P., Tsu, C., Ma, J., Chen, J., Shah, P., Fleming, P., Senn, J. J., Kadambi, V. J., Dick, L., et al. (2016). Proteasome inhibitors bortezomib and carfilzomib used for the treatment of multiple myeloma do not inhibit the serine protease HtrA2/Omi. Toxicology Research 5, 1619-1628.

El Bakkouri, M., Pow, A., Mulichak, A., Cheung, K. L., Artz, J. D., Amani, M., Fell, S., de Koning-Ward, T. F., Goodman, C. D., McFadden, G. I., et al. (2010). The CIp chaperones and proteases of the human malaria parasite Plasmodium falciparum. J Mol Biol 404, 456-477.

Elmore, S. (2007). Apoptosis: a review of programmed cell death. Toxicol Pathol 35, 495-516.

Emsley, P., and Cowtan, K. (2004). Coot: model-building tools for molecular graphics. Acta crystallographica Section D, Biological crystallography 60, 2126-2132.

Engel, M., Longden, J., Ferkinghoff-Borg, J., Robin, X., Saginc, G., and Linding, R. (2018). Bowhead: Bayesian modelling of cell velocity during concerted cell migration. PLoS Comput Biol 14, e100590.

Gersch, M., Famulla, K., Dahmen, M., Gobl, C., Malik, I., Richter, K., Korotkov, V. S., Sass, P., Rubsamen-Schaeff, H., Madl, T., et al. (2015). AAA+ chaperones and acyldepsipeptides activate the CIpP protease via conformational control. Nat Commun 6, 6320.

Goodreid, J. D., Janetzko, J., Santa Maria, J. P., Jr., Wong, K. S., Leung, E., Eger, B. T., Bryson, S., Pai, E. F., Gray-Owen, S. D., Walker, S., et al. (2016). Development and Characterization of Potent Cyclic Acyldepsipeptide Analogues with Increased Antimicrobial Activity. J Med Chem 59, 624-646.

Gribun, A., Kimber, M. S., Ching, R., Sprangers, R., Fiebig, K. M., and Houry, W. A. (2005). The CIpP double ring tetradecameric protease exhibits plastic ring-ring interactions, and the N termini of its subunits form flexible loops that are essential for CIpXP and CIpAP complex formation. The Journal of biological chemistry 280, 16185-16196.

Ingvarsson, H., Mate, M. J., Hogbom, M., Portnoi, D., Benaroudj, N., Alzari, P. M., Ortiz-Lombardia, M., and Unge, T. (2007). Insights into the inter-ring plasticity of caseinolytic proteases from the X-ray structure of Mycobacterium tuberculosis CIpP1. Acta crystallographica Section D, Biological crystallography 63, 249-259.

Kang, S. G., Maurizi, M. R., Thompson, M., Mueser, T., and Ahvazi, B. (2004). Crystallography and mutagenesis point to an essential role for the N-terminus of human mitochondrial CIpP. J Struct Biol 148, 338-352.

Kang, S. G., Ortega, J., Singh, S. K., Wang, N., Huang, N. N., Steven, A. C., and Maurizi, M. R. (2002). Functional proteolytic complexes of the human mitochondrial ATP-dependent protease, hCIpXP. The Journal of biological chemistry 277, 21095-21102.

Kim, D. Y., and Kim, K. K. (2008). The structural basis for the activation and peptide recognition of bacterial CIpP. J Mol Biol 379, 760-771.

Kimber, M. S., Yu, A. Y., Borg, M., Leung, E., Chan, H. S., and Houry, W. A. (2010). Structural and theoretical studies indicate that the cylindrical protease CIpP samples extended and compact conformations. Structure 18, 798-808.

Kroemer, G., and Reed, J. C. (2000). Mitochondrial control of cell death. Nat Med 6, 513-519.

Landes, T., and Martinou, J. C. (2011). Mitochondrial outer membrane permeabilization during apoptosis: the role of mitochondrial fission. Biochim Biophys Acta 1813, 540-545.

Lee, B. G., Park, E. Y., Lee, K. E., Jeon, H., Sung, K. H., Paulsen, H., Rubsamen-Schaeff, H., Brotz-Oesterhelt, H., and Song, H. K. (2010). Structures of CIpP in complex with acyldepsipeptide antibiotics reveal its activation mechanism. Nat Struct Mol Biol 17, 471-478.

Lee, C. D., Sun, H. C., Hu, S. M., Chiu, C. F., Homhuan, A., Liang, S. M., Leng, C. H., and Wang, T. F. (2008). An improved SUMO fusion protein system for effective production of native proteins. Protein Sci 17, 1241-1248.

Leung, E., Datti, A., Cossette, M., Goodreid, J., McCaw, S. E., Mah, M., Nakhamchik, A., Ogata, K., El Bakkouri, M., Cheng, Y. Q., et al. (2011). Activators of cylindrical proteases as antimicrobials: identification and development of small molecule activators of CIpP protease. Chem Biol 18, 1167-1178.

Li, D. H., Chung, Y. S., Gloyd, M., Joseph, E., Ghirlando, R., Wright, G. D., Cheng, Y. Q., Maurizi, M. R., Guarne, A., and Ortega, J. (2010). Acyldepsipeptide antibiotics induce the formation of a structured axial channel in CIpP: A model for the CIpX/CIpA-bound state of CIpP. Chem Biol 17, 959-969.

Liu, K., Ologbenla, A., and Houry, W. A. (2014). Dynamics of the CIpP serine protease: a model for self-compartmentalized proteases. Crit Rev Biochem Mol Biol 49, 400-412.

Lo, J. H., Baker, T. A., and Sauer, R. T. (2001). Characterization of the N-terminal repeat domain of Escherichia coli CIpA-A class I Clp/HSP100 ATPase. Protein Science 10, 551-559.

Lowth, B. R., Kirstein-Miles, J., Saiyed, T., Brötz-Oesterhelt, H., Morimoto, R. I., Truscott, K. N., and Dougan, D. A. (2012). Substrate recognition and processing by a Walker B mutant of the human mitochondrial AAA+ protein CLPX. Journal of Structural Biology 179, 193-201.

Mcllwain, D. R., Berger, T., and Mak, T. W. (2013). Caspase functions in cell death and disease. Cold Spring Harb Perspect Biol 5, a008656.

Mylonas, E., and Svergun, D. I. (2007). Accuracy of molecular mass determination of proteins in solution by small-angle X-ray scattering. Journal of Applied Crystallography 40, s245-s249.

Olivares, A. O., Baker, T. A., and Sauer, R. T. (2016). Mechanistic insights into bacterial AAA+ proteases and protein-remodelling machines. Nat Rev Microbiol 14, 33-44.

Otwinowski, Z., and Minor, W. (1997). Processing of X-ray Diffraction Data Collected in Oscillation Mode. In Macromolecular Crystallography, part A, C. W. Carter Jr., and R. M. Sweet, eds. (New York: Academic Press), pp. 307-326.

Petoukhov, M. V., Franke, D., Shkumatov, A. V., Tria, G., Kikhney, A. G., Gajda, M., Gorba, C., Mertens, H. D., Konarev, P. V., and Svergun, D. I. (2012). New developments in the ATSAS program package for small-angle scattering data analysis. J Appl Crystallogr 45, 342-350.

Pettersen, E. F., Goddard, T. D., Huang, C. C., Couch, G. S., Greenblatt, D. M., Meng, E. C., and Ferrin, T. E. (2004). UCSF Chimera—a visualization system for exploratory research and analysis. J Comput Chem 25, 1605-1612.

Schmitz, K. R., Carney, D. W., Sello, J. K., and Sauer, R. T. (2014). Crystal structure of Mycobacterium tuberculosis CIpP1P2 suggests a model for peptidase activation by AAA+ partner binding and substrate delivery. Proceedings of the National Academy of Sciences 111, E4587-E4595.

Selleck, W., and Tan, S. (2008). Recombinant protein complex expression in E. coli. Curr Protoc Protein Sci Chapter 5, Unit 5 21.

Szczepanowska, K., Maiti, P., Kukat, A., Hofsetz, E., Nolte, H., Senft, K., Becker, C., Ruzzenente, B., Hornig-Do, H. T., Wibom, R., et al. (2016). CLPP coordinates mitoribosomal assembly through the regulation of ERAL1 levels. EMBO J 35, 2566-2583.

Wojtyra, U. A., Thibault, G., Tuite, A., and Houry, W. A. (2003). The N-terminal zinc binding domain of CIpX is a dimerization domain that modulates the chaperone function. The Journal of biological chemistry 278, 48981-48990.

Ye, F., Zhang, J., Liu, H., Hilgenfeld, R., Zhang, R., Kong, X., Li, L., Lu, J., Zhang, X., Li, D., et al. (2013). Helix unfolding/refolding characterizes the functional dynamics of Staphylococcus aureus CIp protease. The Journal of biological chemistry 288, 17643-17653.

Zeiler, E., List, A., Alte, F., Gersch, M., Wachtel, R., Poreba, M., Drag, M., Groll, M., and Sieber, S. A. (2013). Structural and functional insights into caseinolytic proteases reveal an unprecedented regulation principle of their catalytic triad. Proceedings of the National Academy of Sciences of the United States of America 110, 11302-11307.

Zhang, J., Ye, F., Lan, L., Jiang, H., Luo, C., and Yang, C. G. (2011). Structural switching of Staphylococcus aureus CIp protease: a key to understanding protease dynamics. The Journal of biological chemistry 286, 37590-37601.

1. A method for treating a subject having cancer, comprising administering a therapeutically effective amount of an acyldepsipeptide (ADEP) analog to the subject in need thereof.

2. The method of claim 1 , wherein the ADEP analog is ADEP-01, ADEP-02, ADEP-03, ADEP-04, ADEP-05, ADEP-06, ADEP-07, ADEP-08, ADEP-09, ADEP-10, ADEP-11, ADEP-12, ADEP-13, ADEP-14, ADEP-15, ADEP-16, ADEP-17, ADEP-18, ADEP-19, ADEP-20, ADEP-21, ADEP-22, ADEP-23, ADEP-24, ADEP-25, ADEP-26, ADEP-27, ADEP-28, ADEP-29, ADEP-30, ADEP-31, ADEP-32, ADEP-33, ADEP-34, ADEP-35, ADEP-36, ADEP-37, ADEP-38, ADEP-39, ADEP-40, ADEP-41, ADEP-42, ADEP-43, ADEP-44, ADEP-45, or ADEP-46.

3. The method of claim 1, wherein the ADEP analog is ADEP-01 , ADEP-02, ADEP-04, ADEP-06, ADEP-10, ADEP-13, ADEP-14, ADEP-15, ADEP-17, ADEP-20, ADEP-25, ADEP-28, ADEP-29, ADEP-30, ADEP-32, ADEP-37, ADEP-38, or ADEP-41.

4. The method of claim 1, wherein the ADEP analog is ADEP-14, ADEP-17, ADEP-28, ADEP-29, ADEP-30, ADEP-32, ADEP-37, or ADEP-38.

5. The method of claim 1, wherein the ADEP analog activates protease activity of human mitochondrial CIpP (HsCIpP), and wherein the protease activity is at least 0.87 as measured by the relative degradation (RD) index at 25 μM of the ADEP analog, optionally at least 0.9, optionally at least 0.95.

6. The method of claim 1, wherein the protease activity is at least 0.74 as measured by the RD index at 5 μM of the ADEP analog, optionally at least 0.8, optionally at least 0.85.

7. The method of claim 1, wherein the protease activity is at least 0.2 as measured by the RD index at 1 μM of the ADEP analog, optionally at 0.5, optionally at least 0.75, optionally at least 0.8.

8. The method of claim 1, wherein the cancer is breast cancer, prostate cancer, colon cancer, liver cancer, uterus cancer, thyroid cancer, lung cancer, lymph nodes cancer, bladder cancer, ovary cancer, stomach cancer, testis cancer, brain cancer, skin cancer, pancreatic cancer, acute myeloid leukemia, kidney cancer, cervical cancer, osteosarcoma, or neuroblastoma.

9. The method of claim 1, wherein the cancer is breast cancer, kidney cancer, cervical cancer, osteosarcoma, or neuroblastoma.

10. The method of claim 1, wherein the cancer is metastatic.

11. The method of claim 10, wherein the metastatic cancer is breast cancer.

12. The method of claim 1, wherein the subject is human.

13. The method of claim 1, wherein the ADEP analog is administered subcutaneously, intraperitoneally, intravenously, topically, or orally.

14. A compound for use in treating a subject having a cancer, wherein the compound is a therapeutically effective amount of an acyldepsipeptide (ADEP) analog.

15. The compound of claim 14, wherein the ADEP analog is ADEP-01, ADEP-02, ADEP-03, ADEP-04, ADEP-05, ADEP-06, ADEP-07, ADEP-08, ADEP-09, ADEP-10, ADEP-11, ADEP-12, ADEP-13, ADEP-14, ADEP-15, ADEP-16, ADEP-17, ADEP-18, ADEP-19, ADEP-20, ADE P-21, ADEP-22, ADEP-23, ADEP-24, ADEP-25, ADEP-26, ADEP-27, ADEP-28, ADEP-29, ADEP-30, ADEP-31, ADEP-32, ADEP-33, ADEP-34, ADEP-35, ADEP-36, ADEP-37, ADEP-38, ADEP-39, ADEP-40, ADEP-41, ADEP-42, ADEP-43, ADEP-44, ADEP-45, or ADEP-46.

16. The compound of claim 14, wherein the ADEP analog is ADEP-01, ADEP-02, ADEP-04, ADEP-06, ADEP-10, ADEP-13, ADEP-14, ADEP-15, ADEP-17, ADEP-20, ADEP-25, ADEP-28, ADEP-29, ADEP-30, ADEP-32, ADEP-37, ADEP-38, or ADEP-41.

17. The compound of claim 14, wherein the ADEP analog is ADEP-14, ADEP-17, ADEP-28, ADEP-29, ADEP-30, ADEP-32, ADEP-37, or ADEP-38.

18. The compound of claim 14, wherein the ADEP analog activates protease activity of human mitochondrial CIpP (HsCIpP), and wherein the protease activity is at least 0.87 as measured by the relative degradation (RD) index at 25 μM of the ADEP analog, optionally at least 0.9, optionally at least 0.95.

19. The compound of claim 14, wherein the protease activity is at least 0.74 as measured by the RD index at 5 μM of the ADEP analog, optionally at least 0.8, optionally at least 0.85.

20. The compound of claim 14, wherein the protease activity is at least 0.2 as measured by the RD index at 1 μM of the ADEP analog, optionally at 0.5, optionally at least 0.75, optionally at least 0.8.

21. The compound of claim 14, wherein the cancer is breast cancer, prostate cancer, colon cancer, liver cancer, uterus cancer, thyroid cancer, lung cancer, lymph nodes cancer, bladder cancer, ovary cancer, stomach cancer, testis cancer, brain cancer, skin cancer, pancreatic cancer, acute myeloid leukemia, kidney cancer, cervical cancer, osteosarcoma, or neuroblastoma.

22. The compound of claim 14, wherein the cancer is metastatic.

23-26. (canceled)

27. An acyldepsipeptide (ADEP) analog, wherein the ADEP analog is ADEP-01, ADEP-02, ADEP-03, ADEP-04, ADEP-05, ADEP-06, ADEP-07, ADEP-08, ADEP-09, ADEP-10, ADEP-11, ADEP-12, ADEP-13, ADEP-14, ADEP-15, ADEP-16, ADEP-17, ADEP-18, ADEP-19, ADEP-20, ADEP-21, ADEP-22, ADEP-23, ADEP-24, ADEP-25, ADEP-26, ADEP-27, ADEP-28, ADEP-29, ADEP-30, ADEP-31, ADEP-32, ADEP-33, ADEP-34, ADEP-35, ADEP-36, ADEP-37, ADEP-38, ADEP-39, ADEP-40, ADEP-41, ADEP-42, ADEP-43, ADEP-44, ADEP-45, or ADEP-46, or a variant or a derivative thereof.
